SEQUENCE LISTING (57 sequences)

SEQ ID NO: 1 - DNA, 2219 bp, Zea mays
  source 1..2219: mol_type = unassigned DNA
gccgatcacaggatgacaaccgggtcccgcgattgtctggccccgcgggacagtgggcaatgtgcccgtggttgcggtgagaaaaggtcccggccggtacgggaattcaaattgggaggcgggtgcgggccctcgctgcgcgggcagaatcgctgatatgcccttgggccacgccttccttcttatcccctgcgcctgccctcccctcctgcagcgcccgtcgcctccctttccccttccctccctccctctccccgcgggccgccagaggcaatttcgccctcgattcgaccccgattcgtccgcccgcgaccgggattccctccgccaagcgcggatctgaacgctgcgctccgtgtcgccgctcgaatccgccatgaagggggccaaatccaagggcgccgccaaggccgatgccaagtaagaaaaacgaatctccggttctcttcttccttgttcgtcccgtggatctgttccgtggcgtccgaagcattcgatttctgttattatgacatccgccgccgcgagtagattttgacctttttttttcccgtcgtcgttgttttgctgtcgctgtctctgtcgtcgtcgtcgtcgactcgattgctttggcatctgcaggctggcggtgaagagcaagggggcggagaagcccgccaagggcaggaaagggaaggccggcaaggaccccaacaagcctaagagggctcccagcgctttcttcgtgttcatgtgagatgcgacttttttcgtcgtctctttttttttgctatatacttatttctggatgcgactgttgattcgatctatttttggttgcctttttatacagggaggaatttcgcaaggagttcaaggagaagaaccctaagaacaagtctgtcgctgcggtaagaaccctgagagctgctttatgaccggccccatattattactatctactttgacttttcccttaatgacgacttattatttgatttactcgtcacgattcccctctcctggtcgaacttttcaggtggggaaagctgctggcgacaggtggaaatccctgagcgagtcggtaagctccatcttctgtactaaagtagtagttgattggactagaaatctcgtgctgattaattgttttacgcgtgcgtttgtgtggattgtaggacaaggctccctatgtagccaaggctaacaagctcaagctcgagtacaacaaggccatcgctgcctacaacaagggcgaggtagggaaactgatgcttcaattgcgttgttacttaatgtgtatatagactgctcctgctggtcatttattcaaatacgtatgcttactgaaccatgtgttgtttgttcagagcactgcagctaagaaggctcctgccaaggaggaagaggaggaagatgaagaggagtctgacaagtccaagtcggaggtcaatgacgaggatgatgaagagggtagtgaggaggtatacaaaatctattcctgtcttctccatattttttccttagacaaaataccaattaatccaggatacatagcatgctcatgcattaagcaagttgctaaatttatcttcgaccacaatgtagacctagtagtattgtattggattccatcaatgcaaaacatatgcagatttgcatatgcaaccctcttttgcaagtcctcaatatgagcatttgtttggaacacggtgcattggccttaggaagtctttaaaaatatgtcttccctctgcttgcaggatgaagatgatgacgagtgatggagctcctcgagacaatggaccgtgcttcatccaacaatggagcggctacacaaggccccgtggcgatcacaaaaaaggagcctatatccatgtactagaattattcagtttcactccacatcgtgatgttttatttttacttttgtcgtgctataacggatagcgctcctcgttggcgccactggcgggtggttctgcctctggtctggtgtgtttgtgtgtggtcacacttgccagccagcagaggcgcctgcttgtacctagattactgtttccattgtcgtcatcatcggctaacattgtcataatgtcagtttgggtaatgttagattaagtaattattgtgttctgtattcttgtttgttgcccttcttttgtgtgtttttgttattgttggtctgtttccattgttgttgctttggcattgtcagtgcccgccagccctgaaaattg

SEQ ID NO: 2 - DNA, 663 bp, Zea mays
  source 1..663: mol_type = unassigned DNA
atgaagggggccaaatccaagggcgccgccaaggccgatgccaagctggcggtgaagagcaagggggcggagaagcccgccaagggcaggaaagggaaggccggcaaggaccccaacaagcctaagagggctcccagcgctttcttcgtgttcatggaggaatttcgcaaggagttcaaggagaagaaccctaagaacaagtctgtcgctgcggtggggaaagctgctggcgacaggtggaaatccctgagcgagtcggacaaggctccctatgtagccaaggctaacaagctcaagctcgagtacaacaaggccatcgctgcctacaacaagggcgagagcactgcagctaagaaggctcctgccaaggaggaagaggaggaagatgaagaggagtctgacaagtccaagtcggaggtcaatgacgaggatgatgaagagggtagtgaggaggtatacaaaatctattcctgtcttctccatattttttccttagacaaaataccaattaatccaggatacatagcatgttcatgcattaagcaagttgctaaatttatcttcgaccacaatgtagacctagtagtattgtattggattccatcaatgcaaaacatatgcagatttgcatatgcaaccctcttttgcaagtcctcaatatga

SEQ ID NO: 3 - protein, 220 aa, Zea mays
MKGAKSKGAAKADAKLAVKSKGAEKPAKGRKGKAGKDPNKPKRAPSAFFVFMEEFRKEFKEKNPKNKSVAAVGKAAGDRWKSLSESDKAPYVAKANKLKLEYNKAIAAYNKGESTAAKKAPAKEEEEEDEEESDKSKSEVNDEDDEEGSEEVYKIYSCLLHIFSLDKIPINPGYIACSCIKQVAKFIFDHNVDLVVLYWIPSMQNICRFAYATLFCKSSI

SEQ ID NO: 4 - DNA, 3282 bp, synthetic construct
  misc_feature 1..3282: note = Description of Artificial Sequence: Synthetic polynucleotide
  source 1..3282: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 5 - DNA, 4224 bp, synthetic construct
  misc_feature 1..4224: note = Description of Artificial Sequence: Synthetic polynucleotide
  source 1..4224: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatcccccgggatggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 6 - DNA, 3663 bp, synthetic construct
  misc_feature 1..3663: note = Description of Artificial Sequence: Synthetic polynucleotide
  source 1..3663: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 7 - DNA, 3648 bp, synthetic construct
  misc_feature 1..3648: note = Description of Artificial Sequence: Synthetic polynucleotide
  source 1..3648: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 8 - DNA, 2961 bp, synthetic construct
  misc_feature 1..2961: note = Description of Artificial Sequence: Synthetic polynucleotide
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 9 - DNA, 2961 bp, synthetic construct
  misc_feature 1..2961: note = Description of Artificial Sequence: Synthetic polynucleotide
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 10 - DNA, 2655 bp, synthetic construct
  misc_feature 1..2655: note = Description of Artificial Sequence: Synthetic polynucleotide
  source 1..2655: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 11 - DNA, 2655 bp, synthetic construct
  misc_feature 1..2655: note = Description of Artificial Sequence: Synthetic polynucleotide
  source 1..2655: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 12 - DNA, 2679 bp, synthetic construct
  misc_feature 1..2679: note = Description of Artificial Sequence: Synthetic polynucleotide
  source 1..2679: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 13 - DNA, 2949 bp, synthetic construct
  misc_feature 1..2949: note = Description of Artificial Sequence: Synthetic polynucleotide
  source 1..2949: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 14 - DNA, 3018 bp, synthetic construct
  misc_feature 1..3018: note = Description of Artificial Sequence: Synthetic polynucleotide
  source 1..3018: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 15 - DNA, 2676 bp, synthetic construct
  misc_feature 1..2676: note = Description of Artificial Sequence: Synthetic polynucleotide
  source 1..2676: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 16 - DNA, 1104 bp, Triticum aestivum
  source 1..1104: mol_type = unassigned DNA
atggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttag

SEQ ID NO: 17 - DNA, 2040 bp, Zea mays
  source 1..2040: mol_type = unassigned DNA
atggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 18 - DNA, 1485 bp, Zea mays
  source 1..1485: mol_type = unassigned DNA
atggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatga

SEQ ID NO: 19 - DNA, 1470 bp, Zea mays
  source 1..1470: mol_type = unassigned DNA
atggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtag

SEQ ID NO: 20 - DNA, 783 bp, synthetic construct
  misc_feature 1..783: note = Description of Artificial Sequence: Synthetic polynucleotide
  source 1..783: mol_type = other DNA
atggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattga

SEQ ID NO: 21 - DNA, 783 bp, synthetic construct
  misc_feature 1..783: note = Description of Artificial Sequence: Synthetic polynucleotide
  source 1..783: mol_type = other DNA
atggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattga

SEQ ID NO: 22 - DNA, 477 bp, synthetic construct
  misc_feature 1..477: note = Description of Artificial Sequence: Synthetic polynucleotide
  source 1..477: mol_type = other DNA
atggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactga

SEQ ID NO: 23 - DNA, 477 bp, synthetic construct
  misc_feature 1..477: note = Description of Artificial Sequence: Synthetic polynucleotide
  source 1..477: mol_type = other DNA
atggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactga

SEQ ID NO: 24 - DNA, 501 bp, synthetic construct
  misc_feature 1..501: note = Description of Artificial Sequence: Synthetic polynucleotide
  source 1..501: mol_type = other DNA
atggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattga

SEQ ID NO: 25 - DNA, 771 bp, synthetic construct
  misc_feature 1..771: note = Description of Artificial Sequence: Synthetic polynucleotide
  source 1..771: mol_type = other DNA
atggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattag

SEQ ID NO: 26 - DNA, 840 bp, synthetic construct
  misc_feature 1..840: note = Description of Artificial Sequence: Synthetic polynucleotide
  source 1..840: mol_type = other DNA
atggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactga

SEQ ID NO: 27 - DNA, 498 bp, synthetic construct
  misc_feature 1..498: note = Description of Artificial Sequence: Synthetic polynucleotide
  source 1..498: mol_type = other DNA
atggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtga

SEQ ID NO: 28 - protein, 367 aa, Triticum aestivum
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDALLMESFSDIDLEEFADAFGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 29 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVAGGSASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 30 - protein, 494 aa, Zea mays
MDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRHRWTGRYEAHLWDNSCRREGQSRKGRQVYLGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSSAAAAGGRSSKALESPPSGSLDGGGGMPVVEGSTAPPLFIPVKYDQQQQEYLSMLALQHHHQQQQAGNLLQGPLVGFGGLYSSGVNLDFANSHGTAAPSSMAHHCYANGTASASHEHQHQHQMQQGGENETQPQPQQSSSSCSSLPFATPVAFNGSYESSITAAGPFGYSYPNVAAFQTPIYGME

SEQ ID NO: 31 - protein, 489 aa, Zea mays
MDMDMSSAYPHHWLSFSLSNNYHHGLLEAFSNSSGTPLGDEPGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQVYLGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGATGRAAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAAMVSGSAGYEGSTGDNGTWVTTTITSSNTGTAPHYYNYLFGME

SEQ ID NO: 32 - protein, 260 aa, synthetic construct
  REGION 1..260: note = Description of Artificial Sequence: Synthetic polypeptide
MESGSGTAAGSGYVYRQPGSTRWNPTAEQLSLLREIYYRNGLRTPTADEIRQISSKLSRYGKIEGKNVYNWFQNRRAREKRKQRLSTIGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKILQLFPLHNNEDQTLIKSDKEIYCLGSCEKKMDLSPLGHSGSQRASALDLCLSLGNESCGLHDN

SEQ ID NO: 33 - protein, 260 aa, synthetic construct
  REGION 1..260: note = Description of Artificial Sequence: Synthetic polypeptide
MESGSGTAAGSGYVYRQSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNEDQTLIKSDKEIYCLGSCEKKMDLSPLGHSGSQRASALDLCLSLGNESCGLHDN

SEQ ID NO: 34 - protein, 158 aa, synthetic construct
  REGION 1..158: note = Description of Artificial Sequence: Synthetic polypeptide
MVSKGEEVIKEFPGSTRWNPTAEQLSLLREIYYRNGLRTPTADEIRQISSKLSRYGKIEGKNVYNWFQNRRAREKRKQRLSTIGCGSAGSAAGSGEFRHVKTLPLFPLHNNEDSGSLDLELELGFASATAAATSGSQRASALDLCLSLGNESCGLHDN

SEQ ID NO: 35 - protein, 158 aa, synthetic construct
  REGION 1..158: note = Description of Artificial Sequence: Synthetic polypeptide
MVSKGEEVIKEFSGSTRWNPTAEQLSLLKELYYRNGIRTPSADQIRQISARLSRYGKIEGKNVFYWFQNHKARERQKKRLSTVGCGSAGSAAGSGEFRHVKTLPLFPLHNNEDSGSLDLELELGFASATAAATSGSQRASALDLCLSLGNESCGLHDN

SEQ ID NO: 36 - protein, 166 aa, synthetic construct
  REGION 1..166: note = Description of Artificial Sequence: Synthetic polypeptide
MVSKGEEVIKEFSGSTRWNPTAEQLSLLKELYYRNGIRTPSADQIRQISARLSRYGKIEGKNVFYWFQNHKARERQKKRLSTVGCGSAGSAAGSGEFRHVKTLPLFPLHNNVETLPLFPEDSGSLDLELELGFASATAAATSGSQRASALDLCLSLGNESCGLHDN

SEQ ID NO: 37 - protein, 256 aa, synthetic construct
  REGION 1..256: note = Description of Artificial Sequence: Synthetic polypeptide
MVSKGEEVIKEFSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNVETLPLFPEDSGSLDLELELGFASATAAATSGSQRASALDLCLSLGNESCGLHDN

SEQ ID NO: 38 - protein, 279 aa, synthetic construct
  REGION 1..279: note = Description of Artificial Sequence: Synthetic polypeptide
MVSKGEEVIKEFSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNVETLPLFPEDSGSLDLELELGFASGGSAGSVDTLPLFPREAAGSGEFATAAATSGSQRASALDLCLSLGNESCGLHDN

SEQ ID NO: 39 - protein, 164 aa, synthetic construct
  REGION 1..164: note = Description of Artificial Sequence: Synthetic polypeptide
MVSKGEEVIKEFSGSTRWNPTAEQVKVLREIYYRAGLRTPSADQIRQISSQLKRYGKVEGKNVFYWFQNHKARERQKKRLSTVGSAGSAAGSGEFRHVKTLPLFPLHNNEDSGSGLDLDLNLELRLSATAAATSGSQRASALDLDLELRLGFALGNESCGLHDN

SEQ ID NO: 40 - DNA, 6186 bp, synthetic construct
  misc_feature 1..6186: note = Description of Artificial Sequence: Synthetic polynucleotide
  source 1..6186: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttaga

SEQ ID NO: 41 - DNA, 7128 bp, synthetic construct
  misc_feature 1..7128: note = Description of Artificial Sequence: Synthetic polynucleotide
  source 1..7128: mol_type = other DNA
gatcccccgggatggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttg

SEQ ID NO: 42 - DNA, 6567 bp, synthetic construct
  misc_feature 1..6567: note = Description of Artificial Sequence: Synthetic polynucleotide
  source 1..6567: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaa

SEQ ID NO: 43 - DNA, 6552 bp, synthetic construct
  misc_feature 1..6552: note = Description of Artificial Sequence: Synthetic polynucleotide
  source 1..6552: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtaga

SEQ ID NO: 44 - DNA, 5865 bp, synthetic construct
  misc_feature 1..5865: note = Description of Artificial Sequence: Synthetic polynucleotide
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaa

SEQ ID NO: 45 - DNA, 5865 bp, synthetic construct
  misc_feature 1..5865: note = Description of Artificial Sequence: Synthetic polynucleotide
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaa

SEQ ID NO: 46 - DNA, 5559 bp, synthetic construct
  misc_feature 1..5559: note = Description of Artificial Sequence: Synthetic polynucleotide
  source 1..5559: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactgaa

SEQ ID NO: 47 - DNA, 5559 bp, synthetic construct
  misc_feature 1..5559: note = Description of Artificial Sequence: Synthetic polynucleotide
  source 1..5559: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactgaa

SEQ ID NO: 48 - DNA, 5583 bp, synthetic construct
  misc_feature 1..5583: note = Description of Artificial Sequence: Synthetic polynucleotide
  source 1..5583: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattgaa

SEQ ID NO: 49 - DNA, 5853 bp, synthetic construct
  misc_feature 1..5853: note = Description of Artificial Sequence: Synthetic polynucleotide
  source 1..5853: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattaga

SEQ ID NO: 50 - DNA, 5922 bp, synthetic construct
  misc_feature 1..5922: note = Description of Artificial Sequence: Synthetic polynucleotide
  source 1..5922: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactgaa

SEQ ID NO: 51 - DNA, 5580 bp, synthetic construct
  misc_feature 1..5580: note = Description of Artificial Sequence: Synthetic polynucleotide
  source 1..5580: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtgaa

SEQ ID NO: 52 - DNA, 11544 bp, synthetic construct
  misc_feature 1..11544: note = Description of Artificial Sequence: Synthetic polynucleotide
  source 1..11544: mol_type = other DNA
tttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggtctcggcgcgccggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtttaaacgaagcaaacatggcatctagcatggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcggacggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggagagaccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttggttccattcttttgtttgtctgccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgtacaccataacagaaagtagtgacaagtgttggccaaggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacccataaaaaaggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggca

SEQ ID NO: 53 - DNA, 3852 bp, synthetic construct
  misc_feature 1..3852: note = Description of Artificial Sequence: Synthetic polynucleotide
  source 1..3852: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtcgtcaaaagacctttttaatttctactcttgtagatctcgtcacgattcccctctccggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 54 - DNA, 10427 bp, synthetic construct
  misc_feature 1..10427: note = Description of Artificial Sequence: Synthetic polynucleotide
  source 1..10427: mol_type = other DNA
actgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggcgcgccgatatc

SEQ ID NO: 55 - DNA, 11516 bp, synthetic construct
  misc_feature 1..11516: note = Description of Artificial Sequence: Synthetic polynucleotide
  source 1..11516: mol_type = other DNA
gggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacctgcaggcaagcaagcatgaatgcctgggcgcgccggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtttaaacgaagcaaacatggcatctagcatggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcggacggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatccc

SEQ ID NO: 56 - DNA, 3852 bp, synthetic construct
  misc_feature 1..3852: note = Description of Artificial Sequence: Synthetic polynucleotide
  misc_feature 1..3852: note = Plasmid pGEP1067 sgRNA m7GEP22
  source 1..3852: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtcgtcaaaagacctttttaatttctactcttgtagatttcagagcactgcagctaagaggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 57 - protein, 25 aa, unidentified
  REGION 1..25: note = Description of Unknown: WRC domain conserved motif
  VARIANT 2..10: note = Any amino acid
  VARIANT 12..21: note = Any amino acid
  VARIANT 23..24: note = Any amino acid
CXXXXXXXXXCXXXXXXXXXXCXXH

(19) United States
(12) Patent Application Publication
(10) Pub. No.: US 2026/0226490 A1
(43) Pub. Date: Aug. 6, 2026

(54) METHOD FOR RAPID GENOME MODIFICATION IN RECALCITRANT PLANTS

(71) Applicant: KWS SAAT SE & Co. KGaA, Einbeck (DE)

(72) Inventor: Ling MENG, Saint Louis, MO (US)

(73) Assignee: KWS SAAT SE & Co. KGaA, Einbeck (DE)

(21) Appl. No.: 19/657,487

(22) Filed: Apr. 24, 2026

Related U.S. Application Data
(62) Division of application No. 17/800,696, filed on Feb. 26, 2021, now Pat. No. 12,637,685.
(60) Provisional application No. 62/982,979, filed on Feb. 28, 2020.

(51) Int. Cl. C12N 15/82

(52) U.S. Cl. CPC C12N 15/8262

(57) ABSTRACT

The present invention provides a method for plant genome modification, preferably for the targeted modification of at least one genomic target sequence, for obtaining at least one modified cell, wherein modification of a cell is achieved by providing a genome modification or editing system together with at least one regeneration booster, or a combination of regeneration boosters, which is/are transiently active in the cell and/or with at least one epigenetically regulating chemical. Preferably, the effector molecules are introduced by means of particle bombardment. Furthermore, the modified plant cell is regenerated to obtain a plant, which inherits the modification to its progeny. In addition, methods, tools, constructs and strategies are provided to effectively modify the genome of a plant cell or at least one genomic target site in a plant cell, to obtain said modified cell and to regenerate a plant tissue, organ, plant or seed from the modified cell. Finally, the present invention also relates to an improved method of regenerating a plant tissue, organ or a plant from a single plant cell.

### CROSS-REFERENCE TO RELATED APPLICATIONS

This application is a divisional of U.S. application Ser. No. 17/800,696, filed Aug. 18, 2022, which is a U.S. National Phase of International Patent Application No. PCT/EP2021/054805, filed on Feb. 26, 2021, which claims priority to U.S. Provisional Application No. 62/982,979, filed Feb. 28, 2020. The entire contents of these applications are incorporated herein by reference in their entirety.

### SEQUENCE LISTING

The instant application contains a Sequence Listing which has been submitted electronically in XML format and is hereby incorporated by reference in its entirety. Said XML copy, created on Apr. 14, 2026, is named 245761_000270_SL.xml and is 239,810 bytes in size.

### TECHNICAL FIELD

The present invention relates to the field of plant regeneration from single cell origin, in particular in combination with genome engineering or gene editing of a plant cell. The present invention provides a method, wherein modification of a cell is achieved by providing a genome modification or editing system together with at least one regeneration booster, or a combination of regeneration boosters, which is/are transiently active in the cell and/or with at least one epigenetically regulating chemical. Preferably, the effector molecules are introduced by means of particle bombardment. Furthermore, the modified plant cell is regenerated to obtain a plant, which inherits the modification to its progeny. In addition, methods, tools, constructs and strategies are provided to effectively modify the genome of a plant cell or at least one genomic target site in a plant cell, to obtain said modified cell and to regenerate a plant tissue, organ, plant or seed from the modified cell. Finally, the present invention also relates to an improved method of regenerating a plant tissue, organ or a plant from a single plant cell.

### BACKGROUND OF INVENTION

To cope with the increasing challenges of climate change, food safety and a growing world population, traditional plant breeding, usually being rather time consuming, has to be supported by new techniques of molecular biology to provide new crop plants having desired traits in a safe manner, but needing less development time.

Having more and more potentially suitable site-specific nuclease tools at hand, transformation or transfection and subsequent regeneration are still the major bottleneck technologies for plant genome engineering, such as transgene insertion or genome editing (GE). To obtain a modified plant, the two events have to fall on the same cell. Some plants or plant genotypes are particularly recalcitrant to transformation and/or subsequent regeneration, which makes it very challenging to introduce traits using GE approaches and to propagate them. Up to date, particle bombardment and Agrobacterium-mediated biomolecule delivery are the most efficient methods for plant transformation. In agrobacterial transformation, the Agrobacteria first find the suitable cells and attach to the plant cell walls, which is generally referred to as “inoculation”. Following the inoculation, the Agrobacteria are growing with plant cells under suitable conditions for a period of time—from several hours to several days—to allow T-DNA transfer. Agrobacterium-plant interaction, plant tissue structure, plant cell type, etc. constrain agrobacterial transformation. Limited by plant cell susceptibility and accessibility, it is generally believed that Agrobacterium-mediated transformation is plant species, plant tissue-type and plant cell-type dependent. Conversely, based on physical forces, particle bombardment is—at least in theory—plant species and plant cell-type independent, and is able to transform any cells when appropriate pressure is applied. Still, many plant cells, in particular plant cells freshly isolated from a plant depending on the developmental stage and the tissue they are derived from, suffer severe stress or even cell death when physically bombarded with micro- or nanoparticles of various kinds. Further, bombardment may be associated with low transformation and/or integration frequencies also caused by severe cell damage or rupture. However, physical bombardment per se offers great advantages as it is easy, rapid and versatile and allows for transient and stable expression of the inserted molecules, if desired. Potentially toxic chemicals needed for transfection, or bacterial transformations can be avoided.

It is generally believed that transformed cells are less regenerative than wild type cells. These circumstances may result in poor rates of genome modification such as gene editing in view of the fact that the transformed/transfected material may not be viable enough after the introduction of the GE tools. For example, transformed cells are susceptible to programmed cell death due to the presence of foreign DNA inside of these cells. In addition, stresses arisen from delivery—e.g. bombardment damage—may trigger a cell death as well. Therefore, promoting cell proliferation is especially essential for the regeneration of the transformed cells.

Plant regeneration relies on cell proliferation and development. Addition to direct shoot and embryo culture, organogenesis and embryogenesis are the two alternative plant regeneration pathways that involve re-programming and activating the proliferation of somatic cells. Organogenesis and embryogenesis are through organ (e.g. shoot) and embryo formation respectively, and eventually develop into a complete plantlet.

Genome modification efficiency is controlled largely by host cell statuses. The cells undergoing rapid cell-division, are the most suitable recipients for genetic modification. Promoting cell division may therefore increase DNA accessibility during DNA replication and division process, and thus increase genetic modification efficiency. In order to stimulate cell division and regeneration, so-called regeneration boosters may be co-delivered into the cell with the genome modification tools. However, a continuous activity of the regeneration boosters can negatively impact differentiation and subsequent development of transformed plants. The latter point is caused by the fact that naturally occurring booster proteins are usually transcription factors guiding the progression of cell differentiation at different positions in a precise manner and thus have central roles in plant development.

As disclosed in Lowe et al. (Plant Cell, 2016, 28 (9)) there is a problem associated with the use of naturally occurring regeneration boosters in artificial settings of plant genome modifications: the usually growth-stimulating effect of regeneration boosters-if not as precisely controlled as in the natural environment, where the transcription factors are only expressed in a tightly controlled spatio-temporal manner, the ectopic expression of regeneration boosters used in plant genome modification easily leads to pleiotropic effects on plant growth and fertility. These uncertainties and negative effects are, however, not desired for targeted genome editing. To address this problem, Lowe et al. suggests a rather cumbersome technique of integrating and later on inactivating booster activity by removal of the relevant expression cassettes. Another approach is described by Lowe et al. (In Vitro Cellular & Developmental Biology—Plant 54 (8) 2018), which relies on specific promoters, that are active only in certain tissues.

In summary, efficient genome modification and subsequent regeneration of plants, in particular recalcitrant plants or plant genotypes, is limited by several factors. At first, an efficient way to introduce a desired modification into a target cell has to be established. Notably, when the tools for genome modification are introduced, the target cell may suffer stress or damage from which it may not be able to recover. If the cell carrying the desired modification does survive, regeneration of a plant from single cell origin is often difficult. This problem can be addressed by co-introduction of regeneration boosters, which stimulate cell proliferation in the early stages of regeneration. A continuous and uncontrolled activity of the boosters, however, negatively affects the differentiation and hampers further development into a plant. Furthermore, pleiotropic effects may impact plant growth and fertility.

In order to overcome the above described problems, novel techniques to efficiently modify plant genomes and to regenerate plants, in particular plants carrying a desired modification, are required. In particular, such methods should be applicable to recalcitrant plants/plant genotypes, which are difficult to transform and/or regenerate. It was thus an object of the present invention to provide means and methods to achieve rapid and highly efficient genome modification with single-cell origin directly in recalcitrant elite plant lines. It should be feasible to regenerate plants from the modified cells by reliable differentiation without adverse effects on further development. The modified T0 plants should be transgene-free, fertile and the modifications should be fully inherited to the T1 progeny.

### SUMMARY OF THE INVENTION

In a first aspect the present invention provides a method for plant genome modification, preferably for the targeted modification of at least one genomic target sequence, for obtaining at least one modified cell wherein the method comprises the following steps:
(a) providing at least one plant cell or a single plant cell;
(b) introducing into the at least one plant cell or the single plant cell:
   (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same;
   (ii) at least one regeneration booster, or a sequence encoding the same and/or at least one epigenetically regulating chemical, wherein the at least one regeneration booster is transiently present, transiently active or transiently expressed in the plant cell;
   (iii) and, optionally at least one repair template, or a sequence encoding the same; and
(c) cultivating the at least one plant cell or the single plant cell under conditions allowing the expression and/or assembly and/or activation of the at least one genome modification system, preferably the at least one genome editing system, and, optionally of the at least one regeneration booster, and optionally of the at least one guide molecule and/or optionally of the at least one repair template; and
(d) obtaining at least one modified plant cell; and/or
(e) obtaining at least one plant tissue, organ, plant or seed regenerated from the at least one modified cell; and
(f) optionally: screening for at least one plant tissue, organ, plant or seed regenerated from the at least one modified cell in the T0 and/or T1 generation carrying a desired targeted modification.

In one embodiment of the various aspects of the present invention, in the method described above, steps (i) and (ii) take place simultaneously or subsequently, for promoting plant cell proliferation and/or to assist in a targeted modification of at least one genomic target sequence.

In a further embodiment of the various aspects of the present invention, in the method according to any of the embodiments above, at least one regeneration booster is introduced in step (ii) and
(a) the regeneration booster(s) promote plant cell proliferation and/or assist in the targeted modification and/or provide a positive selection for at least one transiently transformed cell for genome modification and/or for regeneration into at least one modified plant, and/or
(b) the regeneration booster(s) suppress plant cell differentiation if the cell is stably transformed and provide a negative selection for at least one stably transformed cell for regeneration into one transgenic plant, and/or
(c) the regeneration booster(s) provide a dual selection according to (a) and (b) for at least one transformed cell.

In another embodiment of the various aspects of the present invention, in the method according to any of the embodiments described above, the at least one plant cell is an immature embryo cell or a meristematic cell, in particular a cell of a zygotic or somatic embryo or meristem or the single plant cell is a diploid cell or a haploid cell, preferably a haploid microspore.

In one embodiment of the various aspects of the present invention, in the method according to any of the embodiments described above, the at least one regeneration booster comprises at least one regeneration booster protein (RBP), wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

In another embodiment of the various aspects of the present invention, in the method according to any of the embodiments described above, the at least one regeneration booster further comprises at least one PLT or RKD4, wherein the at least one PLT or RKD4 comprises an amino acid sequence selected from the group consisting of SEQ ID NOs: 28, 29, 30 and 31, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or the at least one PLT or RKD4 is encoded by a sequence selected from the group consisting of SEQ ID NOs: 16, 17, 18 and 19, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

In a further embodiment of the various aspects of the present invention, in the method according to any of the embodiments described above, at least one further regeneration booster is introduced, wherein the further regeneration booster, or the sequence encoding the same is selected from BBM, WUS, WOX, GRF, LEC, or a variant thereof or the further regeneration booster is a regeneration booster as defined in any of the embodiments above, wherein the further regeneration booster is different from the first regeneration booster.

In yet another embodiment of the various aspects of the present invention, in the method according to any of the embodiments described above, the at least one regeneration booster comprises at least one RBP, wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence, and the at least one regeneration booster comprises PLT5, wherein the PLT5 comprises an amino acid sequence of SEQ ID NO: 30, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the PLT5 is encoded by a nucleic acid sequence of SEQ ID NO: 18, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

In a further embodiment of the method according to any of the embodiments described above, at least one epigenetically regulating chemical is introduced in step ii) and the at least one epigenetically regulating chemical is a histone deacetylase inhibitor (HDACI), in particular trichostatin A (TSA) or a TSA like chemical.

In one embodiment of the various aspects of the present invention, in the method according to any of the embodiments described above, the at least one genome modification system, preferably the at least one genome editing system, and the at least one regeneration booster, or the sequences encoding the same and/or the at least one epigenetically regulating chemical, are introduced into the cell by transformation or transfection mediated by biolistic bombardment, Agrobacterium-mediated transformation, micro- or nanoparticle delivery, or by chemical transfection, or a combination thereof, preferably the at least one genome modification system, preferably the at least one genome editing system, and the at least one regeneration booster and/or the at least one epigenetically regulating chemical are introduced by biolistic bombardment, preferably wherein the biolistic bombardment comprises a step of osmotic treatment before and/or after bombardment.

In another embodiment of the various aspects of the present invention, in the method according to any of the embodiments described above, at least one site-directed nuclease, nickase or an inactivated nuclease, or a sequence encoding the same, is introduced and is selected from the group consisting of a CRISPR/Cas system, preferably from a CRISPR/MAD7 system, a CRISPR/Cfp1 system, a CRISPR/MAD2 system, a CRISPR/Cas9 system, a CRISPR/CasX system, a CRISPR/CasY system, a CRISPR/Cas13 system, or a CRISPR/Csm system, a zinc finger nuclease system, a transcription activator-like nuclease system, or a meganuclease system, or any combination, variant, or catalytically active fragment thereof.

In a further embodiment of the various aspects of the present invention, in the method according to any of the embodiments described above, at least one genome editing system is introduced, wherein the at least one genome editing system further comprises at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACF1/ASE deaminase, an ADAT family deaminase, an ADAR2 deaminase, or a PmCDA1 deaminase, a TadA derived deaminase, and/or a transposon, or a sequence encoding the aforementioned at least one enzyme, or any combination, variant, or catalytically active fragment thereof.

In one embodiment of the various aspects of the present invention, in the method according to any of the embodiments described above, at least one genome editing system is introduced, wherein the at least one genome editing system comprises at least one repair template, and the at least one repair template comprises or encodes a double- and/or single-stranded nucleic acid sequence.

In another embodiment of the various aspects of the present invention, in the method according to any of the embodiments described above, the at least one repair template comprises symmetric or asymmetric homology arms and/or the at least one repair template comprises at least one chemically modified base and/or backbone.

In a further embodiment of the various aspects of the present invention, in the method according to any of the embodiments described above, at least one genome editing system is introduced, wherein the at least one genome editing system, and optionally the at least one repair template, or the respective sequences encoding the same, are introduced transiently or stably, or as a combination thereof.

In one aspect, the present invention relates to the use of a regeneration booster or a combination of regeneration boosters, preferably a regeneration booster or combination of regeneration boosters as defined in any of the embodiments described above, in a method for targeted plant genome modification, preferably in a method according to any of the embodiments described above, to
(a) promote plant cell proliferation and/or assist in the targeted modification and/or provide a positive selection for at least one transiently transformed cell for genome modification and/or for regeneration into at least one modified plant, and/or
(b) suppress plant cell differentiation if the cell is stably transformed and provide a negative selection for at least one stably transformed cell for regeneration into one transgenic plant, and/or
(c) provide a dual selection according to (a) and (b) for at least one transformed cell.

In another aspect, the present invention relates to a plant cell, tissue, organ, plant or seed obtainable by or obtained by a method according to any of the embodiments described above.

In one embodiment of the plant cell, tissue, organ, plant or seed described above, the plant cell, tissue, organ, plant or seed originates from a monocotyledonous or a dicotyledonous plant.

In another embodiment of the plant cell, tissue, organ, plant or seed described above, originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant cell, tissue, organ, plant or seed originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

In a further aspect the present invention relates to an expression construct assembly, comprising
(i) at least one vector encoding at least one genome modification system which comprises at least one gene of interest, preferably an exogenous gene of interest, and/or which is preferably at least one genome editing system comprising at least one site-directed nuclease, nickase or an inactivated nuclease of a genome editing system, preferably wherein the genome editing system is as defined in any of the embodiments described above, and
(ii) at least one vector encoding at least one regeneration booster, preferably wherein the regeneration booster or boosters is/are as defined in any of the embodiments described above, and
(iii) optionally, when the at least one site-directed nuclease, nickase or an inactivated nuclease of a genome editing system is a nucleic acid guided nuclease: at least one vector encoding at least one guide molecule guiding the at least one nucleic acid guided nuclease, nickase or an inactivated nuclease to the at least one genomic target site of interest; and
(iv) optionally: at least one vector encoding at least one repair template;
   wherein (i), (ii), (iii), and/or (iv) are encoded on the same, or on different vectors.

In one embodiment of the expression construct assembly described above, the assembly further comprises a vector encoding at least one marker.

In yet another aspect the present invention relates to a plant cell, tissue, organ, plant or seed comprising at least one regeneration booster as described above and further below and/or an expression construct assembly as defined in any of the embodiments described above.

In a further aspect the present invention relates to a method for selecting a modified plant cell, plant tissue, organ, plant or seed, wherein the method comprises the steps (a) to (c) as defined in any of the embodiments of the method described above and further comprises the step:
(d1) screening for at least one plant cell carrying a targeted modification, or
(d2) screening for at least one plant tissue, organ, plant or seed regenerated from at least one modified plant cell in the T0 or T1 generation carrying a desired targeted modification.

In another aspect the present invention relates to a method for regenerating a plant tissue, organ or plant from at least one plant cell or from a single plant cell, wherein the method comprises:
(i) Introducing into the at least one plant cell or the single plant cell at least one regeneration booster, or a sequence encoding the same, wherein the at least one regeneration booster is transiently present, transiently active or transiently expressed in the plant cell, preferably wherein the at least one regeneration booster(s) is/are as defined in any of the embodiments described above, and
(ii) regenerating a plant tissue, organ or plant from at least one plant cell or the single plant cell.

In one embodiment of the method for regenerating a plant tissue, organ or plant from a single plant cell as described above, the at least one plant cell is an immature embryo cell or meristematic cell, in particular a cell of a zygotic or somatic embryo or meristem wherein the single plant cell is a diploid cell or a haploid cell.

In another embodiment of the method for regenerating a plant tissue, organ or plant from a single plant cell as described above, the single plant cell is a haploid microspore.

### BRIEF DESCRIPTION OF THE DRAWINGS

Whenever the Figures show black/white pictures of originally fluorescence images, brighter spots represent the accumulation of respective fluorescent protein.

FIG. 1 shows a genome editing nuclease MAD7 expression construct pGEP837 map. A green fluorescent marker was used in this example (indicated as GEP). Any kind of fluorescent protein-encoding marker gene may be used instead depending on the plant target cell/tissue to be transformed and visualized. MAD7 defines the maize codon-optimized CDS of the Eubacterium rectale CRISPR/MAD7 gene (Inscripta). BdUBI10 defines the Brachypodium Ubiquitin 10 promoter. Tnos defines the nos terminator

FIG. 2 shows a genome editing sgRNA construct pGEP842 map. m7GEP1 defines the crRNA, which target to maize HMG13 gene. ZmUbi1 defines the promoter and intron from maize Ubiquitin 1 gene. Tnos defines the nos terminator.

FIG. 3 shows a maize PLT5 expression construct pABM-BdEF1_ZmPLT5 map. ZMPLT5 is driven by the strong constitutive EF1 gene promoter from Brachypodium (BdEF1).

FIG. 4 shows the KWS_RBP2 expression construct pABM-BdEF1_RBP2 map. KWS_RBP2 is driven by the strong constitutive EF1 promoter from Brachypodium (BdEF1).

FIG. 5 shows the KWS_RBP3 expression construct pABM-BdEF1_RBP3 map. KWS_RBP3 is driven by the strong constitutive EF1 promoter from Brachypodium (BdEF1).

FIG. 6 shows the KWS_RBP4 expression construct pABM-BdEF1_RBP4 map. KWS_RBP4 is driven by the strong constitutive EF1 promoter from Brachypodium (BdEF1).

FIG. 7 shows the KWS_RBP5 expression construct pABM-BdEF1_RBP5 map. KWS_RBP5 is driven by the strong constitutive EF1 promoter from Brachypodium (BdEF1).

FIG. 8 shows the KWS_RBP6 expression construct pABM-BdEF1_RBP6 map. KWS_RBP6 is driven by the strong constitutive EF1 promoter from Brachypodium (BdEF1).

FIG. 9 shows the KWS_RBP7 expression construct pABM-BdEF1_RBP7 map. KWS_RBP7 is driven by the strong constitutive EF1 promoter from Brachypodium (BdEF1).

FIG. 10 shows the KWS_RBP8 expression construct pABM-BdEF1_RBP8 map. KWS_RBP8 is driven by the strong constitutive EF1 promoter from Brachypodium (BdEF1).

FIG. 11 shows how different regeneration boosters stimulate cell proliferation in the scutellum surfaces of maize immature embryos after the co-bombardment of genome editing constructs pGEP837 (FIG. 1) and pGEP842 (FIG. 2) with the boosters and cultured for 4 days in the embryogenic callus induction medium. (A) to (H): the embryos from maize recalcitrant elite 16V-2015 bombarded with the genome editing constructs only (A), or con-bombarded with the boosters of ZmPLT5 and KWS_RBP2 (B), ZmPLT5 and KWS_RBP3 (C), ZmPLT5 and KWS_RBP4 (D), ZmPLT5 and KWS_RBP5 (E), ZmPLT5 and KWS_RBP6 (F), ZmPLT5 and KWS_RBP7 (G), or ZmPLT5 and KWS_RBP8 (H). (I) to (L): the embryos from maize recalcitrant elite 4V-40214 bombarded with the genome editing constructs only (I), or co-bombarded with the boosters of ZmPLT5 and RBP2 (J), ZmPLT5 and RBP5 (K), or ZmPLT5 and RBP8 (L).

FIG. 12 shows how different regeneration boosters promote rapid genome transformation in maize immature embryos after the co-bombardment of genome editing constructs pGEP837 (FIG. 1) and pGEP842 (FIG. 2) with the boosters and cultured for 4 days in the embryogenic callus induction medium. (A) to (C): the embryos from maize recalcitrant elite WA4-29814 bombarded with the genome editing constructs only (A), or co-bombarded with the boosters of ZmPLT5 and KWS_RBP2 (B), or ZmPLT5 and KWS_RBP8 (C). (D) to (G): the embryos from maize recalcitrant elite 4V-40214 bombarded with the genome editing constructs only (D), or co-bombarded with the boosters of ZmPLT5 and KWS RBP2 (E), ZmPLT5 and KWS_RBP5 (F), or ZmPLT5 and KWS_RBP8 (G).

FIG. 13 shows a genome editing nuclease LbCpf1 expression construct pGEP359 map. tDT defines the tdTomato gene. ZmLpCpf1 defines the maize codon-optimized CDS of the Lachnospiraceae bacterium CRISPR/Cpf1 (LbCpf1) gene.

FIG. 14 shows how cell differentiation and subsequent regeneration are negatively impacted by the continuous boosting activity of ZmPLT5 (FIG. 3) and KWS_RBP4 (FIG. 6) as indicated by the co-delivered report gene tDTomato from construct pGEP359 (FIG. 13) in maize elite 4V-40171. tDTomato-negative embryogenic calluses were developed into mature embryos (A), while the embryo maturation were inhibited in the tDTomato-positive calluses in (B) and (C) after incubated in the embryo maturation medium for 10 day. Grey shades of the tissue in (B) and (C) indicate a red coloration, which is due to tDTomato expression.

FIG. 15 shows a genome editing nuclease MAD7 expression construct pGEP1054 map. tdTomato defines tdTomato report gene. MAD7 defines the maize codon-optimized CDS of MAD7 nuclease (Inscripta). BdUBI10 defines the Brachypodium Ubiquitin 10 promoter. Tnos defines the nos terminator.

FIG. 16 shows tDTomato fluorescence images of maize A188 immature embryos 16 hour after bombardment. The immature embryos were bombarded with plasmid pGEP1054 only, which contains CRISPR nuclease MAD7 and tDTomato expression cassettes (A), or co-bombarded with plasmids of pGEP1054 and pGEP842 that contains crRNA m7GEP1 expression cassette (B), or co-bombarded with plasmids of pGEP1054, pGEP842, and booster KWS_RBP2 (C).

FIG. 17 shows SDN-1 efficiency in bombarded maize A188 immature embryos sampled at the time as indicated (Avg of three experiments). The immature embryos were bombarded with 200 ng plasmid pGEP1054 only, which contains the CRISPR nuclease MAD7 and tDTomato expression cassettes (MAD7 only), or co-bombarded with 200 ng of plasmid pGEP1054 and 300 ng of plasmid pGEP842 that contains the guide RNA m7GEP1 expression cassette (MAD7/sgRNA), or co-bombarded with 200 ng of plasmid pGEP1054 and 300 ng of plasmid pGEP842, plus 100 ng of plasmid KWS_RBP2 (MAD7/sgRNA/RBP2). The bombarded embryos were sampled at the time as indicated, and the SDN-1 were analyzed by ddPCR, HPB: hours post bombardment; DPB: days post bombardment.

FIG. 18 shows how different regeneration boosters enhance genome editing in the regenerated T0 plants of maize A188. A: SDN-1 efficiency per regenerated T0 event; B: SDN-1 efficiency per immature embryo (IE) initially used. The regeneration boosters were co-delivered with the genome editing nuclease MAD7 construct pGEP837 (FIG. 1) and the sgRNA construct pGEP842 (FIG. 2) into maize A188 immature embryos by particle bombardment.

FIG. 19 shows the workflow for rapid plant regeneration and genome modification via particle bombardment of immature embryos (IEs) from maize recalcitrant elite 4V-40290. A: The immature embryos (IEs) in osmotic medium for 4 hours and were ready for particle bombardment; B: embryogenic calluses were induced from the scutellum surface of the IEs after co-bombarded with the boost constructs pABM-BdEF1_ZmPLT5 and pABM-BdEF1_RBP8 and cultured on embryogenic callus induction medium for 6 days; (C): embryogenic calluses were matured after cultured on the maturation medium for 10 days; (D): T0 plantlets were developed from the mature embryos after outgrowth on the embryo germination medium for 7 days; (E): T0 plants growing in green house; (F): 14-day-old T1 embryos were germinating on MSO medium for 3 days; G: T1 plants in green house.

FIG. 20 shows how the regeneration boosters promote embryogenic callus induction from maize recalcitrant elite 4V-40171. (A) to (G): images of embryogenic callus induction for 4 days in a callus induction medium from the embryos bombarded with the genome editing constructs pGEP837 and pGEP842 only (A), or co-bombarded with booster KWS_RBP2 (B), KWS_RBP4 (C), KWS_RBP5 (D), ZmPLT5 and KWS_RBP5 (E), ZmPLT5 and KWS_RBP6 (F), or ZmPLT5 and KWS_RBP7 (G). (H) to (K): images of embryogenic callus induction in a callus induction medium for 7 days from the embryos bombarded with the genome editing constructs pGEP837 and pGEP842 only (H) or co-bombarded with boosters of ZmPLT5 and KWS_RBP2 (I), ZmPLT5 and ZmPLT5 and KWS_RBP3 (J), or ZmPLT5 and KWS_RBP4 (K).

FIG. 21 shows embryogenic callus induction and plant regeneration efficiencies from the recalcitrant maize elite 4V-40171 immature embryos after co-bombarded with boosters as indicated. (A) Embryogenic callus induction rates (the numbers of embryos with at least one embryogenic callus from 100 embryos initially used; results recorded 12 days after bombardment; (B) plant regeneration rates (calculated by the total regenerated T0 events from 100 immature embryos initially used).

FIG. 22 shows that the regeneration boosters promote stable transformation of the fluorescent report gene tDTomato in maize recalcitrant elite 4V-40171. (A) Fluorescent images of immature embryos in embryogenic callus induction medium for 8 days after bombarded with tDTomato construct pGEP359 (FIG. 13) only (A), or co-bombarded with boosters ZmPLT5 and KWS_RBP2 (B), ZmPLT5 and KWS_RBP3 (C), ZmPLT5 and KWS_RBP4 (D), ZmPLT5 and KWS_RBP5 (E), ZmPLT5 and KWS_RBP6 (F), or ZmPLT5 and KWS_RBP7 (G). (H) Stable transformation frequency of tDTomato report gene (the numbers of embryos with at least one tDTomato-positive structure from 100 embryos initially used). The results were recorded 8 days after co-bombarded with boosters as indicated.

FIG. 23 shows how different boosters promote genome editing in recalcitrant maize elite 4V-40171. A: SDN-1 efficiency per regenerated T0 event (the number of SDN-1 events per 100 regenerated T0 events); B: SDN-1 efficiency per immature embryo (the number of SDN-1 events per 100 immature embryos initially used). The regeneration boosters were co-delivered with the genome editing constructs pGEP837 (FIG. 1) and the pGEP842 (FIG. 2) into maize immature embryos by particle bombardment.

FIG. 24 shows how regeneration boosters promote embryogenic callus induction from the immature embryos of maize recalcitrant elite 2V-20195. The embryos were cultured in callus induction medium for 8 days after the bombarded with the genome editing construct pGEP837 only (A), or co-bombarded with the boosters of ZmPLT5 and KWS_RBP2 (B), ZmPLT5 and KWS_RBP3 (C), ZmPLT5 and KWS_RBP4 (D), ZmPLT5 and KWS_RBP5 (E), or ZmPLT5 and KWS_RBP6 (F). Images were taken 8 days after bombardment.

FIG. 25 shows embryogenic callus induction and plant regeneration efficiencies from the recalcitrant maize elite 2V-20195 immature embryos after co-bombarded with boosters as indicated. (A) Embryogenic callus induction rates (the numbers of embryos with at least one embryogenic callus from 100 embryos initially used; results recorded after cultured in the medium for 14 days; (B) plant regeneration rates (calculated by the total regenerated T0 events from 100 immature embryos initially used).

FIG. 26 shows how regeneration boosters promote stable transformation of a green fluorescent report gene (GEP) in maize recalcitrant elite 2V-20195. (A) fluorescent images of immature embryos in callus induction medium for 8 days after bombarded with the GEP construct pGEP837 only (A), or co-bombarded with the boosters of ZmPLT5 and KWS_RBP2 (B), ZmPLT5 and KWS_RBP3 (C), ZmPLT5 and KWS_RBP4 (D), ZmPLT5 and KWS_RBP5 (E), or ZmPLT5 and KWS_RBP6 (F). (H) Stable transformation frequency of the GEP report gene in the immature embryos (the numbers of embryos with at least one tDTomato-positive structure from 100 embryos initially used). The results were recorded 14 days after co-bombarded with the boosters as indicated.

FIG. 27 shows how different boosters promote genome editing in recalcitrant maize elite 2V-20195. A: SDN-1 efficiency per regenerated event (SDN-1 events from 100 regenerated T0 events); B: SDN-1 efficiency per immature embryo (SDN-1 events per 100 immature embryos initially used). The regeneration boosters were co-delivered with the genome editing constructs of pGEP837 and pGEP842 into maize immature embryos by particle bombardment.

FIG. 28 shows how different boosters promote genome editing in T₀ plants of maize recalcitrant elite 4V-40290. A: SDN-1 efficiency per regenerated T0 events (the number of SDN-1 T0 events per 100 regenerated T0 events); B: SDN-1 efficiency per embryo (the number of SDN-1 T0 events per 100 immature embryos initially used).

FIG. 29 shows how different boosters enable highly efficient genome editing directly in maize recalcitrant elites.

FIG. 30 shows the edited T0 maize elite plants in green house. The regenerated T0 plants from maize recalcitrant elite 4V-4071 (A), 2V-20195 (B), or 4V-40290 (C).

FIG. 31 shows the T1 progeny of maize recalcitrant elite 4V-4071 growing in green house.

FIG. 32 shows a genome editing guide RNA construct pGEP1067 map. m7GEP22 defines the crRNA, which target to maize HMG13 gene. ZmUbi1 defines the promoter and intron from maize Ubiquitin 1 gene. Tnos defines the nos terminator.

FIG. 33: Regeneration boosters promote genome editing SDN-1 efficiency 48 hours after co-introduced with the editing constructs into corn A188 immature embryos (n=6). A: shows genome editing SDN-1 efficiencies at CRISPR target m7GEP1, or at target m7GEP22 in B. No booster: bombarded with the editing plasmids only (100 ng of plasmid pGEP1054 and 150 ng of plasmid pGEP842 in A or 100 ng of plasmid pGEP1054 and 150 ng of plasmid pGEP1067 in B). The immature embryos were co-bombarded with the same amounts of the CRISPR plasmids as those used in “No booster”, and plus 200 ng of plasmid ZmPLT5 (ZmPLT5) or 100 ng of plasmid KWS_RBP2 (KWS_RBP2), or 200 ng of plasmid ZmPLT5 and 100 ng of plasmid KWSRBP2 (PLT5/RBP2). Error bar=standard deviation. The SDN-1 were analyzed by Taqman ddPCR.

FIG. 34: _Trichostatin A (TSA) promotes genome editing SDN-1 efficiency 48 hours after co-introduced with the editing plasmids (n=6). A: shows SDN-1 efficiencies at the CRISPR target m7GEP1 and B shows the SDN-1 efficiencies at target m7GP22. CK: the immature embryos were bombarded with the editing constructs only (100 ng of plasmid pGEP1054 and 150 ng of plasmid pGEP842 in A or 100 ng of plasmid pGEP1054 and 150 ng of plasmid pGEP1067 in B). TSA: the immature embryos were co-bombarded with the editing plasmids and 15 ng of TSA. Error bar=standard deviation. The SDN-1 were analyzed by Taqman ddPCR.

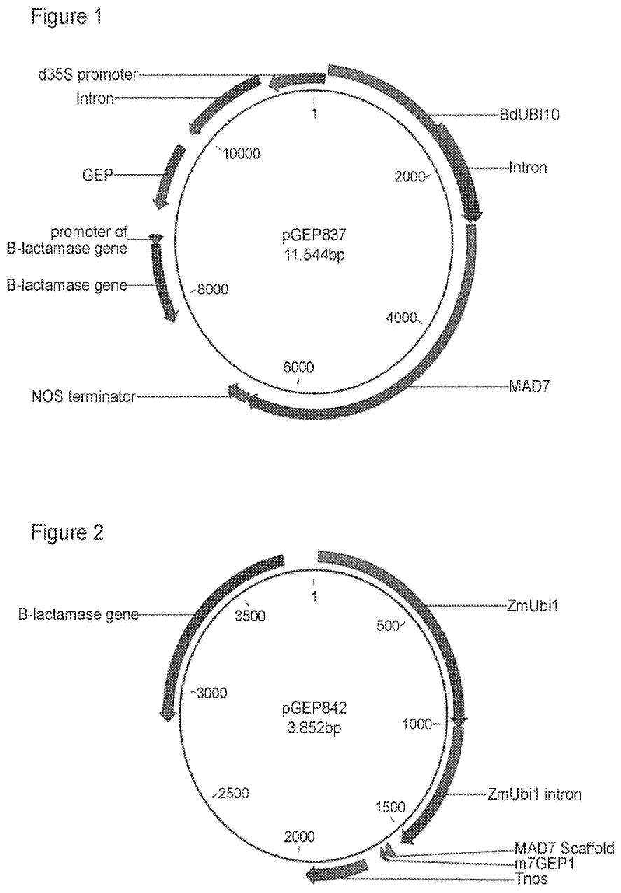

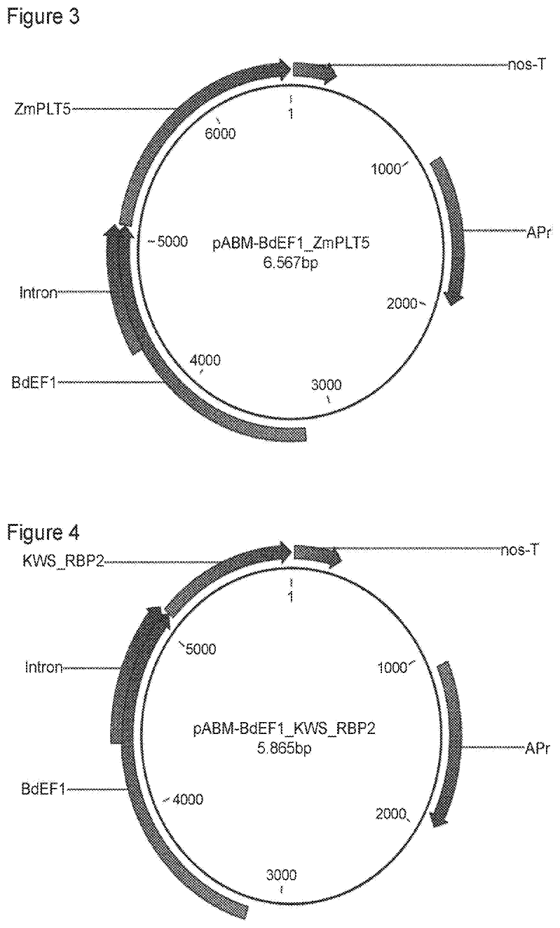

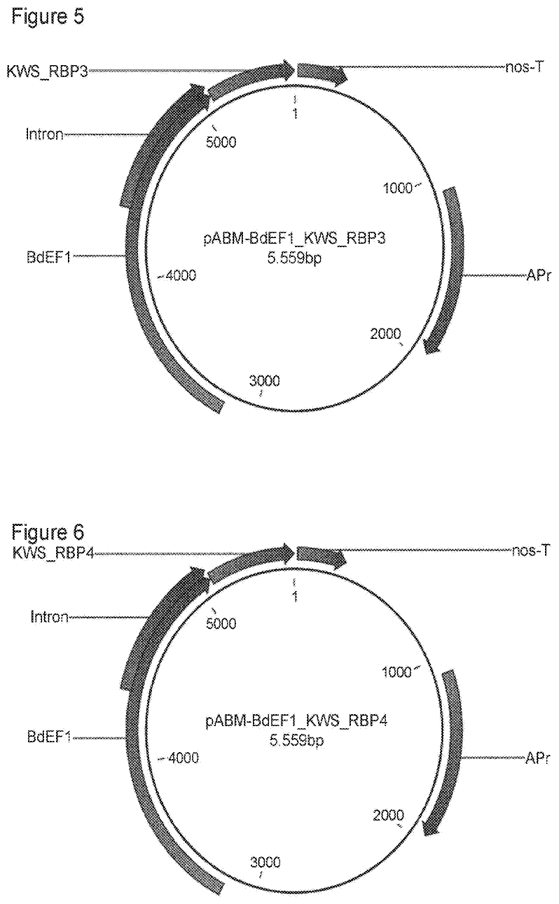

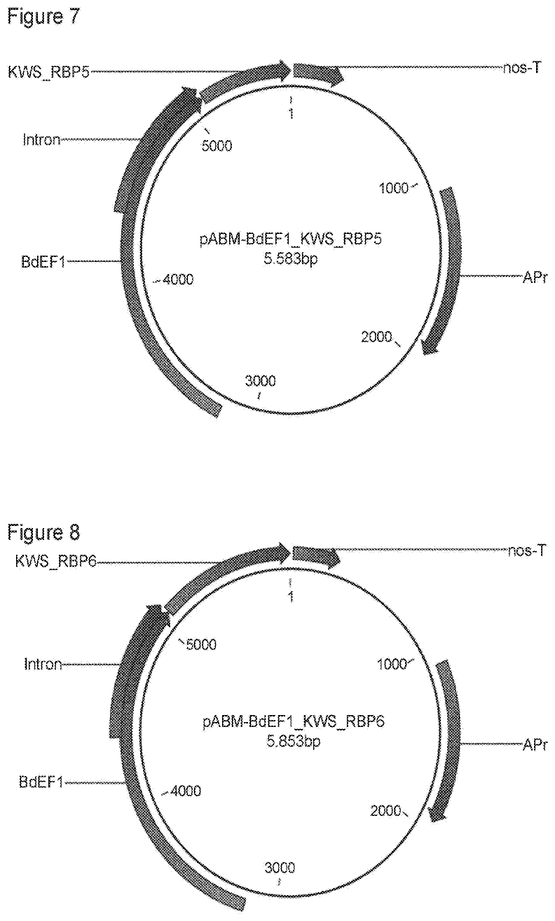

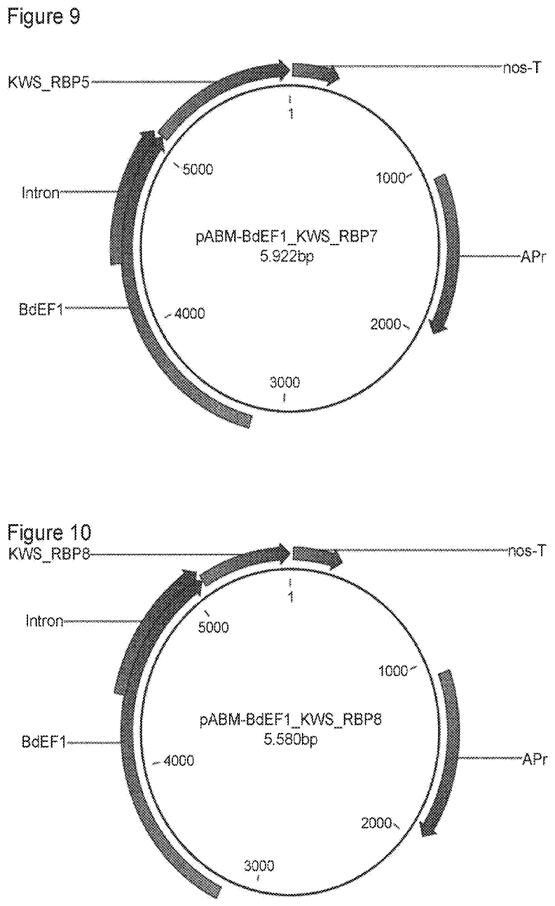

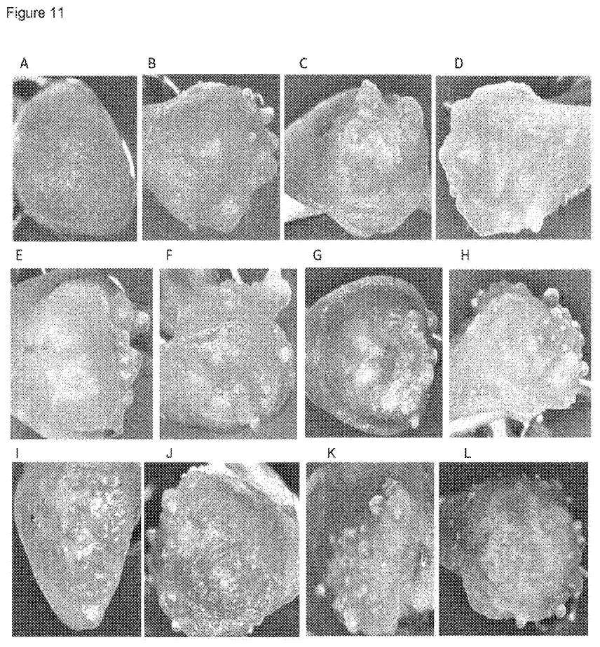

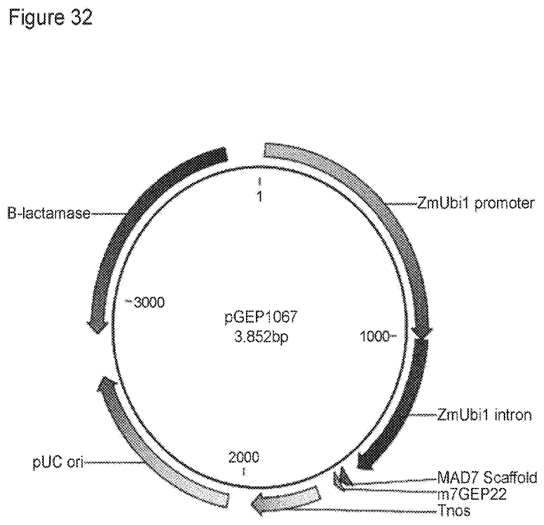

Description of Sequences

| SEQ | |
|---|---|
| ID NO | Brief description |
| 1 | A188-HMG13-WT_Full |
| 2 | Maize_HMG13_CDS_B73 CM000781.3 |
| 3 | Maize_HMG13_protein AFW69593.1 high mobility |
| | group protein1 [Zea mays] |
| 4 | BdEF1_TaRKD4_expression_cassette |
| 5 | BdEF1_ZmPLT3_expression_cassette |
| 6 | BdEF1_ZmPLT5_expression_cassette |
| 7 | BdEF1_ZmPLT7_expression_cassette |
| 8 | BdEF1_KWS_RBP1_expression_cassette |
| 9 | BdEF1_KWS_RBP2_expression_cassette |
| 10 | pABM-BdEF1_RBP3_expression_cassette |
| 11 | pABM-BdEF1_RBP4_Expression_cassette |
| 12 | pABM-BdEF1_RBP5_expression_cassette |
| 13 | pABM-BdEF1_RBP6_expression_cassette |
| 14 | pABM-BdEF1_RBP7_expression_cassette |
| 15 | pABM-BdEF1_RBP8_expression_cassette |
| 16 | TaRKD4_CDS (coding sequence) |
| 17 | ZmPLT3-17207_CDS (coding sequence) |
| 18 | ZmPLT5_CDS (coding sequence) |
| 19 | ZmPLT7_CDS (coding sequence) |
| 20 | RBP1_CDS (coding sequence) |
| 21 | RBP2_CDS (coding sequence) |
| 22 | RBP3_CDS (coding sequence) |
| 23 | RBP4_CDS (coding sequence) |
| 24 | RBP5_CDS (coding sequence) |
| 25 | RBP6_CDS (coding sequence) |
| 26 | RBP7_CDS (coding sequence) |
| 27 | RBP8_CDS (coding sequence) |
| 28 | TaRKD4_PRT (Protein) |
| 29 | ZmPLT3_PRT (Protein) |
| 30 | ZmPLT5_PRT (Protein) |
| 31 | ZmPLT7_PRT (Protein) |
| 32 | RBP1_PRT (Protein) |
| 33 | RBP2_PRT (Protein) |
| 34 | RBP3_PRT (Protein) |
| 35 | RBP4_PRT (Protein) |
| 36 | RBP5_PRT (Protein) |
| 37 | RBP6_PRT (Protein) |
| 38 | RBP7_PRT (Protein) |
| 39 | RBP8_PRT (Protein) |
| 40 | pABM-BdEF1_TaRKD4 |
| 41 | pABM-BdEF1_ZmPLT3 |
| 42 | pABM-BdEF1_ZmPLT5 (FIG. 3) |
| 43 | pABM-BdEF1_ZmPLT7 |
| 44 | pABM-BdEF1_KWS_RBP1 |
| 45 | pABM-BdEF1_KWS_RBP2 (FIG. 4) |
| 46 | pABM-BdEF1_KWS_RBP3 (FIG. 5) |
| 47 | pABM-BdEF1_KWS_RBP4 (FIG. 6) |
| 48 | pABM-BdEF1_KWS_RBP5 (FIG. 7) |
| 49 | pABM-BdEF1_KWS_RBP6 (FIG. 8) |
| 50 | pABM-BdEF1_KWS_RBP7 (FIG. 9) |
| 51 | pABM-BdEF1_KWS_RBP8 (FIG. 10) |
| 52 | pGEP837 (FIG. 1) |
| 53 | pGEP842 (FIG. 2) |
| 54 | pGEP359 (FIG. 13) |
| 55 | pGEP1054 (FIG. 15) |
| 56 | pGEP1067 (FIG. 32) |

### Definitions

Unless defined otherwise, technical and scientific terms used herein have the same meaning as commonly understood by one of ordinary skill in the art to which this invention belongs.

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Usually, base editors are thus used as molecular complex. Base editors, including, for example, CBEs (base editors mediating C to T conversion) and ABEs (adenine base editors mediating A to G conversion), are powerful tools to introduce direct and programmable mutations without the need for double-stranded cleavage (Komor et al., Nature, 2016, 533 (7603), 420-424; Gaudelli et al., Nature, 2017, 551, 464-471). In general, base editors are composed of at least one DNA targeting module and a catalytic domain that deaminates cytidine or adenine. All four transitions of DNA (A→T to G→C and C→G to T→A) are possible as long as the base editors can be guided to the target site. Originally developed for working in mammalian cell systems, both BEs and ABEs have been optimized and applied in plant cell systems. Efficient base editing has been shown in multiple plant species (Zong et al., Nature Biotechnology, vol. 25, no. 5, 2017, 438-440; Yan et al., Molecular Plant, vol. 11, 4, 2018, 631-634; Hua et al., Molecular Plant, vol. 11, 4, 2018, 627-630). Base editors have been used to introduce specific, directed substitutions in genomic sequences with known or predicted phenotypic effects in plants and animals. But they have not been used for directed mutagenesis targeting multiple sites within a genetic locus or several loci to identify novel or optimized traits.

A “CRISPR nuclease”, as used herein, is a specific form of a site-directed nuclease and refers to any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. A variety of different CRISPR nucleases/systems and variants thereof are meanwhile known to the skilled person and include, inter alia, CRISPR/Cas systems, including CRISPR/Cas9 systems (EP2771468), CRISPR/Cpf1 systems (EP3009511B1), CRISPR/C2C2 systems, CRISPR/CasX systems, CRISPR/CasY systems, CRISPR/Cmr systems, CRISPR/MAD systems, including, for example, CRISPR/MAD7 systems (WO2018236548A1) and CRISPR/MAD2 systems, CRISPR/CasZ systems and/or any combination, variant, or catalytically active fragment thereof. A nuclease may be a DNAse and/or an RNAse, in particular taking into consideration that certain CRISPR effector nucleases have RNA cleavage activity alone, or in addition to the DNA cleavage activity.

A “CRISPR system” is thus to be understood as a combination of a CRISPR nuclease or CRISPR effector, or a nickase or a nuclease-dead variant of said nuclease, or a functional active fragment or variant thereof together with the cognate guide RNA (or pegRNA or crRNA) guiding the relevant CRISPR nuclease.

As used herein, the terms “(regeneration) booster”, “booster gene”, “booster polypeptide”, “boost polypeptide”, “boost gene” and “boost factor”, refer to a protein/peptide(s), or a (poly) nucleic acid fragment encoding the protein/polypeptide, which accelerate cell division and cell proliferation and thus cause improved plant regeneration, in particular of transformed or gene edited plant cells, which may be particularly suitable for improving genome engineering, i.e., the regeneration of a modified plant cell after genome engineering. Such protein/polypeptide may increase the capability or ability of a plant cell, preferably derived from somatic tissue, embryonic tissue, callus tissue or protoplast, to regenerate in an entire plant, preferably a fertile plant. Thereby, they may regulate somatic embryo formation (somatic embryogenesis) and/or they may increase the proliferation rate of plant cells. The regeneration of transformed or gene edited plant cells may include the process of somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc. Applications of this process may include: clonal propagation of genetically uniform plant material; elimination of viruses; provision of source tissue for genetic transformation; generation of whole plants from single cells, such as protoplasts; development of synthetic seed technology. Cells derived from competent source tissue may be cultured to form a callus. Further, the term “regeneration booster” may refer to any kind of chemical having a proliferative and/or regenerative effect when applied to a plant cell, tissue, organ, or whole plant in comparison to a no-treated control.

As used herein, “epigenetically regulating chemical” refers to any chemical involved in regulating the epigenetic status of plant cells, e.g., DNA methylation, protein methylation and acetylation. Preferred epigenetically regulating chemicals for use according to the invention are histone deacetylase inhibitors (HDACIs). Histone deacetylase inhibitor (HDACI) refers to any materials that repress histone deacetylase activity as used herein. Such a HDACI may be trichostatin A (TSA), N-Hydroxy-7-(4-dimethylaminobenzoyl)-aminoheptanamide (M344), suberoylanilide hydroxamic acid (SAHA), or others. These HDACIs are selected from hydroxamic acid (HA)-based chemicals, which target to zinc dependent HDACs. TSA suppresses HDAC activities and increases histone acetylation (Yoshida et al, 1995; Finnin et al. 1999).

As used herein, “co-delivery” or “co-deliver” and “co-introduction” or “co-introduce” are used interchangeably. In terms of the present invention, these terms refers to the process, in which at least two different components are delivered into the same plant cell concurrently. Thus, the genome modification components and boost components are introduced together into the same plant cell.

Preferably, both types of components, booster and genes of interest, are introduced via separate constructs.

A “genome” as used herein is to be understood broadly and comprises any kind of genetic information (RNA/DNA) inside any compartment of a living cell. In the context of a “genome modification”, the term thus also includes artificially introduced genetic material, which may be transcribed and/or translated, inside a living cell, for example, an episomal plasmid or vector, or an artificial DNA integrated into a naturally occurring genome.

The term of “genome engineering” as used herein refers to all strategies and techniques for the genetic modification of any genetic information (DNA and RNA) or genome of a plant cell, comprising genome transformation, genome editing, but also including less site-specific techniques, including TILLING and the like. As such, “genome editing” (GE) more specifically refers to a special technique of genome engineering, wherein a targeted, specific modification of any genetic information or genome of a plant cell. As such, the terms comprise gene editing of regions encoding a gene or protein, but also the editing of regions other than gene encoding regions of a genome. It further comprises the editing or engineering of the nuclear (if present) as well as other genetic information of a plant cell, i.e., of intronic sequences, non-coding RNAs, miRNAs, sequences of regulatory elements like promoter, terminator, transcription activator binding sites, cis or trans acting elements. Furthermore, “genome engineering” also comprises an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression.

A “genome modification system” as used herein refers to any DNA, RNA and/or amino acid sequence introduced into the cell, on a suitable vector and/or coated on a particles and/or directly introduced. A “genome editing” system more specifically refers to any DNA, RNA and/or amino acid sequence introduced into the cell, on a suitable vector and/or coated on a particles and/or directly introduced, wherein the “genome editing system” comprises at least one component being, encoding, or assisting a site-directed nuclease, nickase or inactivated variant thereof in modifying and/or repairing a genomic target site.

A “genomic target sequence” as used herein refers to any part of the nuclear and/or organellar genome of a plant cell, whether encoding a gene/protein or not, which is the target of a site-directed genome editing or gene editing experiment.

A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

As used herein “a preselected site”, “predetermined site” or “predefined site” indicates a particular nucleotide sequence in the genome (e.g. the nuclear genome, or the organellar genome, including the mitochondrial or chloroplast genome) at which location it is desired to insert, replace and/or delete one or more nucleotides. The predetermined site is thus located in a “genomic target sequence/site” of interest and can be modified in a site-directed manner using a site- or sequence-specific genome editing system.

The terms “plant”, “plant organ”, or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

The term “plant parts” as used herein includes, but is not limited to, isolated and/or pre-treated plant parts, including organs and cells, including protoplasts, callus, leaves, stems, roots, root tips, anthers, pistils, seeds, grains, pericarps, embryos, pollen, sporocytes, ovules, male or female gametes or gametophytes, cotyledon, hypocotyl, spike, floret, awn, lemma, shoot, tissue, petiole, cells, and meristematic cells.

A “Prime Editing system” as used herein refers to a system as disclosed in Anzalone et al. (2019). Search-and-replace genome editing without double-strand breaks (DSBs) or donor DNA. Nature, 1-1). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations, and is said to introduce off-target mutations which are generally not desired for high precision GE. In contrast, Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid::nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

As used herein, a “regulatory sequence”, or “regulatory element” refers to nucleotide sequences which are not part of the protein-encoding nucleotide sequence, but mediate the expression of the protein-encoding nucleotide sequence. Regulatory elements include, for example, promoters, cis-regulatory elements, enhancers, introns or terminators. Depending on the type of regulatory element it is located on the nucleic acid molecule before (i.e., 5′ of) or after (i.e., 3′ of) the protein-encoding nucleotide sequence. Regulatory elements are functional in a living plant cell.

An “RNA-guided nuclease” is a site-specific nuclease, which requires an RNA molecule, i.e. a guide RNA, to recognize and cleave a specific target site, e.g. in genomic DNA or in RNA as target. The RNA-guided nuclease forms a nuclease complex together with the guide RNA and then recognizes and cleaves the target site in a sequence-dependent matter. RNA-guided nucleases can therefore be programmed to target a specific site by the design of the guide RNA sequence. The RNA-guided nucleases may be selected from a CRISPR/Cas system nuclease, including CRISPR/Cpf1 systems, CRISPR/C2C2 systems, CRISPR/CasX systems, CRISPR/CasY systems, CRISPR/Cmr systems, CRISPR/Cms systems, CRISPR/MAD7 systems, CRISPR/MAD2 systems and/or any combination, variant, or catalytically active fragment thereof. Such nucleases may leave blunt or staggered ends. Further included are nickase or nuclease-dead variants of an RNA-guided nuclease, which may be used in combination with a fusion protein, or protein complex, to alter and modify the functionality of such a fusion protein, for example, in a base editor or Prime Editor.

The terms “SDN-1”, “SDN-2”, and “SDN-3” as used herein are abbreviations for the platform technique “site-directed nuclease” 1, 2, or 3, respectively, as caused by any site directed nuclease of interest, including, for example, Meganucleases, Zinc-Finger Nucleases (ZFNs), Transcription Activator Like Effector Nucleases (TALENs), and CRISPR nucleases. SDN-1 produces a double-stranded or single-stranded break in the genome of a plant without the addition of foreign DNA. A “site-directed nuclease” is thus able to recognize and cut, optionally assisted by further molecules, a specific sequence in a genome or an isolate genomic sequence of interest. For SDN-2 and SDN-3, an exogenous nucleotide template is provided to the cell during the gene editing. For SDN-2, however, no recombinant foreign DNA is inserted into the genome of a target cell, but the endogenous repair process copies, for example, a mutation as present in the template to induce a (point) mutation. In contrast, SDN-3 mechanism use the introduced template during repair of the DNA break so that genetic material is introduced into the genomic material.

A “site-specific nuclease” herein refers to a nuclease or an active fragment thereof, which is capable to specifically recognize and cleave DNA at a certain location. This location is herein also referred to as a “target sequence”. Such nucleases typically produce a double-strand break (DSB), which is then repaired by non-homologous end-joining (NHEJ) or homologous recombination (HR). Site-specific nucleases include meganucleases, homing endonucleases, zinc finger nucleases, transcription activator-like nucleases and CRISPR nucleases, or variants including nickases or nuclease-dead variants thereof.

The terms “transformation”, “transfection”, “transformed”, and “transfected” are used interchangeably herein for any kind of introduction of a material, including a nucleic acid (DNA/RNA), amino acid, chemical, metabolite, nanoparticle, microparticle and the like into at least one cell of interest by any kind of physical (e.g., bombardment), chemical or biological (e.g., Agrobacterium) way of introducing the relevant at least one material.

The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e. a sequence encoding a protein. It can also refer, for example, to a non-protein encoding DNA or RNA sequence, or part of a sequence. Therefore, the term “transgenic” generally implies that the respective nucleic acid or amino acid sequence is not naturally present in the respective target cell, including a plant, plant cell, tissue, organ or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like.

As used herein, the term “transient” implies that effectors, including all kinds of nucleic acid (RNA and/or DNA) and polypeptide-based molecules optionally including chemical carrier molecules, are only temporarily introduced and/or expressed and/or activated and, e.g. afterwards degraded by the cell, whereas “stable” implies that at least one of the effectors is integrated into the nuclear and/or organellar genome of the cell to be modified and therefore inherited to the progeny. “Transient expression” refers to the phenomenon where the transferred protein/polypeptide and/or nucleic acid fragment encoding the protein/polypeptide is expressed, present and/or active transiently in the cells, and turned off and/or degraded shortly with the cell growth. Transient expression thus also implies a stably integrated construct, for example, under the control of an inducible promoter as regulatory element, to regulate expression in a fine-tuned manner by switching expression on or off.

The terms “vector”, or “plasmid (vector)” refer to a construct comprising, inter alia, plasmids or (plasmid) vectors, cosmids, artificial yeast- or bacterial artificial chromosomes (YACs and BACs), phagemides, bacterial phage based vectors, Agrobacterium compatible vectors, an expression cassette, isolated single-stranded or double-stranded nucleic acid sequences, comprising sequences in linear or circular form, or amino acid sequences, viral vectors, viral replicons, including modified viruses, and a combination or a mixture thereof, for introduction or transformation, transfection or transduction into any eukaryotic cell, including a plant, plant cell, tissue, organ or material according to the present disclosure. A “nucleic acid vector, for instance, is a DNA or RNA molecule, which is used to deliver foreign genetic material to a cell, where it can be transcribed and optionally translated. Preferably, the vector is a plasmid comprising multiple cloning sites. The vector may further comprise a “unique cloning site” a cloning site that occurs only once in the vector and allows insertion of DNA sequences, e.g. a nucleic acid cassette or components thereof, by use of specific restriction enzymes. A “flexible insertion site” may be a multiple cloning site, which allows insertion of the components of the nucleic acid cassette according to the invention in an arrangement, which facilitates simultaneous transcription of the components and allows activation of the RNA activation unit.

Whenever the present disclosure relates to the percentage of the homology or identity of nucleic acid or amino acid sequences to each other over the entire length of the sequences to be compared to each other, wherein these identity or homology values define those as obtained by using the EMBOSS Water Pairwise Sequence Alignments (nucleotide) programme nucleic acids or the EMBOSS Water Pairwise Sequence Alignments (protein) programme for amino acid sequences. Those tools provided by the European Molecular Biology Laboratory (EMBL) European Bioinformatics Institute (EBI) for local sequence alignments use a modified Smith-Waterman algorithm (see ebi.ac.uk/Tools/psa/ and Smith, T. F. & Waterman, M. S. “Identification of common molecular subsequences” Journal of Molecular Biology, 1981 147 (1): 195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

### DETAILED DESCRIPTION

The present invention provides methods for rapid and highly efficient genome modification in a single cell as well as regeneration of a plant from a single cell. The invention describes nucleotides/proteins of regeneration boosters and methods to increase regeneration capabilities of plant cells; when co-delivered with genome modification machinery, genome modification is promoted. Using the regeneration boosters or combinations of regeneration boosters, it is possible to positively select transient expression delivery of the boosters and negatively select stable transgenic cells continuously expressing the boosters during regeneration. This “dual selection” allows on the one hand to profit from the improved genome modification efficiency and enhanced proliferation and regeneration provided by the regeneration boosters but on the other hand to exclude plants, which continuously express the boosters negatively impacting differentiation and subsequent development. The modification is stably inherited to the progeny of the regenerated plant while the modification tools are preferably only transiently present or expressed in the cell to be modified and are no longer present in the regenerated plant. Thus, it is possible to obtain a fertile transgene-free plant carrying the modification with minimal somatic variation.

Another type of effectors, which can promote genetic modification and increase genome editing SDN-1 efficiency, are epigenetically regulating chemicals.

The basic structural and functional unit of genetic material is the nucleosome, in which negatively charged DNA wraps around a positively charged histone octamer and associated linker histones. Nucleosome units further fold and pack into chromatin (Andrews, A. J., and Luger, K. 2011. Nucleosome structure(s) and stability: Variations on a theme. Annu. Rev. Biophys. 40:99-117.). DNA accessibility largely depends on compactness of the nucleosomes and chromatins. Chromatin-remodeling enzymes dynamically modify lysine or other amino acids of histones, which cause changes in their charges and interactions with DNA and other proteins, and result in chromatin folding or unfolding (Bannister A J, Kouzarides T. 2011, Regulation of chromatin by histone modifications. Cell Res. 21:381-95.). By adding or removing an acetyl group, acetylation and deacetylation of the lysine residue in histone proteins are often involved in the reversible modulation of chromatin structure in eukaryotes, and mediate chromatin accessibility and gene expression. Histone deacetylases (HDACs) are enzymes that remove acetyl groups from lysine resides on the N-terminal tail of histones, which makes the histone more positively charged, and therefore allows the histone wrap DNA more tightly. And thus, inhibition of HDACs may help chromatin unfolding and enable the DNA to be more accessible.

It is assumed that histone deacetylase inhibitors (HADCIs) will relax plant chromatin structure and promote the DNA accessibility to the genome modification machinery when it is co-delivered with the modification component(s) in the introduced cells, thus consequently promote genome modification, e.g., genome editing efficiency. Described herein are methods to increase genome modification efficiency, in particularly genome editing SDN-1 efficiency in presence of at least one selected epigenetically regulating chemical, preferably histone deacetylase inhibitors (HDACIs).

In one aspect, the present invention provides a method for plant genome modification, preferably for the targeted modification of at least one genomic target sequence, for obtaining at least one modified cell wherein the method comprises the following steps:
(a) providing at least one plant cell or a single plant cell;
(b) introducing into the at least one plant cell or the single plant cell:
   (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same;
   (ii) at least one regeneration booster, or a sequence encoding the same and/or at least one epigenetically regulating chemical, wherein the at least one regeneration booster is transiently present, transiently active or transiently expressed in the plant cell;
   (iii) and, optionally at least one repair template, or a sequence encoding the same; and
(c) cultivating the at least one plant cell or the single plant cell under conditions allowing the expression and/or assembly and/or activation of the at least one genome modification system, preferably the at least one genome editing system, and, optionally of the at least one regeneration booster, and optionally of the at least one guide molecule and/or optionally of the at least one repair template; and
(d) obtaining at least one modified plant cell; and/or
(e) obtaining at least one plant tissue, organ, plant or seed regenerated from the at least one modified cell; and
(f) optionally: screening for at least one plant tissue, organ, plant or seed regenerated from the at least one modified cell in the T0 and/or T1 generation carrying a desired targeted modification.

In the method according to the invention, the use of the regeneration booster(s) provides an increased genome editing efficiency and enhances proliferation of the edited cell to regenerate a plant. The cells, in which the regeneration boosters are only transiently present, transiently active or transiently expressed in the cell, further develop into mature plants.

Transient expression, transient presence or transient activity of the booster(s) in the cell is achieved by introduction of the booster(s) or the nucleic acid sequence encoding the booster(s) into the target cell resulting in gene expression and/or activity without integration of the coding sequence into the genome of the target cell. The regeneration booster genes are expressed transiently, preferably under a strong constitutive promoter and then turned off and/or degraded. For example, the strong constitutive promoter from Brachypodium EF1 gene may be used to drive booster gene expression for transient transformation. The booster genes are translated and active transiently, and turned off and degraded shortly with plant cell development or if the expressing conditions are removed.

The epigenetically regulating chemical may be introduced optionally in addition to the regeneration boosters or it may also be introduced without any regeneration boosters to improve the genome editing efficiency.

In one embodiment, the epigenetically regulating chemical is delivered into explant cells via a pre-treatment of the cells as described further below.

In another embodiment, the epigenetically regulating chemical is co-delivered with the genome editing components into explant cells via co-bombardment. This represents the preferred method, which provides a selective mechanism for the co-delivered cells to achieve genome editing and regeneration.

In yet another embodiment, the epigenetically regulating chemical is co-delivered with the genome editing components and at least one regeneration booster gene construct into explant cells via co-bombardment. This represents a highly preferred method for recalcitrant genotypes, which provides a highly selective mechanism for the delivered cells to achieve genome editing and regeneration.

In another embodiment, the epigenetically regulating chemical is delivered via both, pretreatment and co-bombardment.

In one embodiment of the method described above, steps (i) and (ii) take place simultaneously or subsequently, for promoting plant cell proliferation and/or to assist in a targeted modification of at least one genomic target sequence.

Preferably, all exogenously provided elements or tools of a genome or gene editing system as well as the regeneration booster(s), or sequences encoding the same and/or the epigenetically regulating chemical, and optionally provided repair template sequences are provided either simultaneously or subsequently, wherein the terms simultaneously and subsequently refers to the temporal order of introducing the relevant elements, with the proviso that both simultaneous and subsequent introduction guarantee that one and the same cell will comprise the relevant elements in an active and/or expressible manner. Ultimately, all genome or gene editing system elements are thus physically present in one cell. In particular, the boosters being present and active during genome editing assist in the targeted modification making it more efficient and subsequently enhance proliferation regeneration of the modified cell.

In one embodiment of the method according to any of the embodiments described above, at least one regeneration booster is introduced in step (ii) and
(a) the regeneration booster(s) promote plant cell proliferation and/or assist in the targeted modification and/or provide a positive selection for at least one transiently transformed cell for genome modification and/or for regeneration into at least one modified plant, and/or
(b) the regeneration booster(s) suppress plant cell differentiation if the cell is stably transformed and provide a negative selection for at least one stably transformed cell for regeneration into one transgenic plant, and/or
(c) the regeneration booster(s) provide a dual selection according to (a) and (b) for at least one transformed cell.

As explained above, the regeneration booster(s) increase genome editing efficiency and at the same time promote proliferation of transiently transformed cells while suppressing plant cell differentiation of stably transformed cells. This dual selection advantageously allows to regenerate a modified plant, which does not pass on the genome editing tools to its progeny but merely carries the desired modification.

In one embodiment of the method according to any of the embodiments described above, the at least one plant cell is an immature embryo cell or a meristematic cell, in particular a cell of a zygotic or somatic embryo or meristem or the single plant cell is a diploid cell or a haploid cell, preferably a haploid microspore.

An immature embryo represents a suitable state of development, into which a targeted genome modification may be introduced and a modified plant may be regenerated from the embryo cell. Either zygotic embryos or somatic somatic embryos derived from any type of plant material, shoot, hypocotyl, cotyledon, stem, leave, petiole, root, flower, gametophyte or part thereof may be used.

The plant cells into which genome modification/editing components have been introduced are cultured under suitable conditions allowing the genome modification to occur in said plant cell by activity of the genome modification/editing components in the presence of the at least one regeneration booster and/or the at least one epigenetically regulating chemical. The genetically modified plant cells can be regenerated into a whole plant. Thus, the genetic modification of a plant cell is followed by a step of regenerating a plant.

The method according to the invention may therefore comprise the following steps:
1. Prepare plant cells as part of preferably immature embryos (IEs), either zygotic embryos or somatic embryos.
2. The delivery of genome modification components and regeneration boosters and/or the at least one epigenetically regulating chemical e.g. via particle bombardment into the plant cells.

During regeneration, single or multiple cells proliferate and develop into tissues, organs, and eventually entire plants. Accordingly, the regeneration of plant cells as part of the immature embryos (IEs), either zygotic embryos or somatic embryos after the delivery of genome modification/editing components, may comprise the steps of:
a. Plant cell proliferation and embryogenic callus induction for 1-4 weeks, preferably 1-3 weeks, most preferably 7-14 day;
b. Plant organ/embryo development of the calluses from (a) for 1-4 weeks, preferably 1-3 weeks, most preferably 10-18 days
c. Plantlet development of the organs/embryos from (b) for 1-4 weeks, preferably 4-10 days

Certain regeneration booster sequences, usually representing transcription factors active during various stages of plant development and also known as morphogenic regulators in plants, are known for long, including the Wuschel (WUS) and babyboom (BBM) class of boosters (Mayer, K. F. et al. Role of WUSCHEL in regulating stem cell fate in the Arabidopsis shoot meristem. Cell 95, 805-815 (1998); Yadav, R. K. et al. WUSCHEL protein movement mediates stem cell homeostasis in the Arabidopsis shoot apex. Genes Dev 25, 2025-2030 (2011); Laux, T., Mayer, K. F., Berger, J. & Jürgens, G. The WUSCHEL gene is required for shoot and floral meristem integrity in Arabidopsis. Development 122, 87-96 (1996); Leibfried, A. et al. WUSCHEL controls meristem function by direct regulation of cytokinin-inducible response regulators. Nature 438, 1172-1175 (2005); for BBM: Hofmann, A Breakthrough in Monocot Transformation Methods, The Plant Cell, Vol. 28: 1989, September 2016). Others, including the RKD (including TaRKD4) and LEC family of transcription factors have been steadily emerging and are meanwhile known to the skilled person (Hofmann, A Breakthrough in Monocot Transformation Methods The Plant Cell, Vol. 28: 1989, September 2016; New Insights into Somatic Embryogenesis: LEAFY COTYLEDON1, BABY BOOM1 and WUSCHEL-RELATED HOMEOBOX4 Are Epigenetically Regulated in Coffea canephora, PLos one August 2013, vol. 8 (8), e72160; LEAFY COTYLEDON1-CASEIN KINASE I-TCP15-PHYTOCHROME INTERACTING FACTOR4 Network Regulates Somatic Embryogenesis by Regulating Auxin Homeostasis Plant Physiology_, December 2015, Vol. 169, pp. 2805-2821; A. Cagliari et al. New insights on the evolution of Leafy cotyledon1 (LEC1) type genes in vascular plants Genomics 103 (2014) 380-387, U.S. Pat. No. 6,825,397B1; U.S. Pat. No. 7,960,612B2, WO2016146552A1).

The Growth-Regulating Factor (GRF) family of transcription factors, which is specific to plants, is also known to the skilled person. At least nine GRF polypeptides have been identified in Arabidopsis thaliana (Kim et al. (2003) Plant J 36:94-104), and at least twelve in Oryza sativa (Choi et al. (2004) Plant Cell Physiol 45 (7): 897-904). The GRF polypeptides are characterized by the presence in their N-terminal half of at least two highly conserved domains, named after the most conserved amino acids within each domain: (i) a QLQ domain (InterPro accession IPR014978, PFAM accession PF08880), where the most conserved amino acids of the domain are Gln-Leu-Gln; and (ii) a WRC domain (InterPro accession IPR014977, PFAM accession PF08879), where the most conserved amino acids of the domain are Trp-Arg-Cys. The WRC domain further contains two distinctive structural features, namely, the WRC domain is enriched in basic amino acids Lys and Arg, and further comprises three Cys and one His residues in a conserved spacing (CX9CX10CX2H (SEQ ID NO: 57)), designated as the Effector of Iranscription (ET) domain (Ellerstrom et al. (2005) Plant Molec Biol 59:663-681). The conserved spacing of cysteine and histidine residues in the ET domain is reminiscent of zinc finger (zinc-binding) proteins. In addition, a nuclear localisation signal (NLS) is usually comprised in the GRF polypeptide sequences.

Another class of potential regeneration boosters, yet not studied in detail for their function in artificial genome/gene editing, is the class of PLETHORS (PLT) transcription factors (Aida, M., et al. (2004). The PLETHORA genes mediate patterning of the Arabidopsis root stem cell niche. Cell 119:109-120; Mähönen, A. P., et al. (2014). PLETHORA gradient formation mechanism separates auxin responses. Nature 515:125-129). Organ formation in animals and plants relies on precise control of cell state transitions to turn stem cell daughters into fully differentiated cells. In plants, cells cannot rearrange due to shared cell walls. Thus, differentiation progression and the accompanying cell expansion must be tightly coordinated across tissues. PLETHORA (PLT) transcription factor gradients are unique in their ability to guide the progression of cell differentiation at different positions in the growing Arabidopsis thaliana root, which contrasts with well-described transcription factor gradients in animals specifying distinct cell fates within an essentially static context. To understand the output of the PLT gradient, the gene set transcriptionally controlled by PLTs were studied and it was revealed how the PLT gradient can regulate cell state by region-specific induction of cell proliferation genes and repression of differentiation. Moreover, PLT targets include major patterning genes and autoregulatory feedback components, enforcing their role as master regulators of organ development (Santuari et al., 2016, DOI: doi.org/10.1105/tpc.16.00656). PLT, also called AIL (AINTEGUMENT-LIKE) genes, are members of the AP2 family of transcriptional regulators. Members of the AP2 family of transcription factors play important roles in cell proliferation and embryogenesis in plants (El Ouakfaoui, S. et al., (2010) Control of somatic embryogenesis and embryo development by AP2 transcription factors. PLANT MOLECULAR BIOLOGY 74 (4-5): 313-326.). PLT genes are expressed mainly in developing tissues of shoots and roots, and are required for stem cell homeostasis, cell division and regeneration, and for patterning of organ primordia. PLT family comprises an AP2 subclade of six members. Four PLT members, PLT1/AIL3 PLT2/, AIL4, PLT3/A/L6, and BBM/PLT4/AIL2, are expressed partly overlap in root apical meristem (RAM) and required for the expression of QC (quiescent center) markers at the correct position within the stem cell niche. These genes function redundantly to maintain cell division and prevent cell differentiation in root apical meristem. Three PLT genes, PLT3/AIL6, PLT5/AIL5, and PLT7/AIL7, are expressed in shoot apical meristem (SAM), where they function redundantly in the positioning and outgrowth of lateral organs. PLT3, PLT5, and PLT7, regulate de novo shoot regeneration in Arabidopsis by controlling two distinct developmental events. PLT3, PLT5, and PLT7 required to maintain high levels of PIN1 expression at the periphery of the meristem and modulate local auxin production in the central region of the SAM which underlies phyllotactic transitions. Cumulative loss of function of these three genes causes the intermediate cell mass, callus, to be incompetent to form shoot progenitors, whereas induction of PLT5 or PLT7 can render shoot regeneration in a hormone-independent manner. PLT3, PLT5, PLT7 regulate and require the shoot-promoting factor CUP-SHAPED COTYLEDON2 (CUC2) to complete the shoot-formation program. PLT3, PLT5, and PLT7, are also expressed in lateral root founder cells, where they redundantly activate the expression of PLT1 and PLT2, and consequently regulate lateral root formation.

Regeneration boosters derived from naturally occurring transcription factors, as, for example, BBM or WUS, and variants thereof, may have the significant disadvantage that uncontrolled activity in a plant cell over a certain period of time will have deleterious effects on a plant cell. Therefore, the present inventors conducted a series of in silico work to create fully artificial regeneration booster proteins after a series of multiple sequence alignment, domain shuffling, truncations and codon optimization for various target plants. By focusing on core consensus motifs, it was an object to identify new variants of regeneration boosters not occurring in nature that are particularly suitable for use in plant regeneration from single cell origin, in particular in the context of genome modifications and gene editing. Various gymnosperm sequences occurring in different species presently not considered as having a regeneration booster activity of described booster genes and proteins were particularly considered in the design process of the new booster sequences.

Based on this work, it was now found that specific regeneration boosters (cf. SEQ ID NOs: 20 to 27, 32 to 39, regeneration booster proteins 1 to 8 (RBP1-8)), as well as certain modified regeneration boosters naturally acting as transcription factors (SEQ ID NOs: 16 to 19, 28 to 31) artificially created perform particularly well in combination with the methods disclosed herein, as they promote regeneration and additionally have the capacity to improve genome or gene editing efficiencies. Further, the artificially created and then stepwise selected and tested regeneration boosters do not show pleiotropic effects and are particularly suitable to be used during any kind of genome modification and/or gene editing. The boosters or booster combinations described herein allow to positively select transient expression delivery and negatively select stable transgenic cells continuously expressing the boosters during regeneration.

In one embodiment of the method according to any of the embodiments described above, the at least one regeneration booster comprises at least one RBP, wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

The artificial regeneration boosters RBP1 to RBP8 may be used in any combination with each other or and/or in combination with other boosters. RBP1 may be used in combination with RBP2, or in combination with RBP3, or in combination with RBP4, or in combination with RBP5, or in combination with RBP6, or in combination with RBP7, or in combination with RBP8. RBP2 may be used in combination with RBP3, or in combination with RBP4, or in combination with RBP5, or in combination with RBP6, or in combination with RBP7, or in combination with RBP8. RBP3 may be used in combination with RBP4, or in combination with RBP5, or in combination with RBP6, or in combination with RBP7, or in combination with RBP8. RBP4 may be used in combination with RBP5, or in combination with RBP6, or in combination with RBP7, or in combination with RBP8. RBP5 may be used in combination with RBP6, or in combination with RBP7, or in combination with RBP8. RBP6 may be used in combination with RBP7, or in combination with RBP8. RBP8 may also be used in combination with RBP7. The artificial regeneration boosters RBP1 to RBP8 may also be used in any combination of two, three, four, five, six, seven or all of the boosters RBP1 to RBP8.

Preferably, RBP1 to RBP8 or any combination thereof are combined with one of a PLT or RKD booster. RBP1 may be used in combination with PLT3, or in combination with PLT5, or in combination with PLT7, or in combination with RKD4. RBP2 may be used in combination with PLT3, or in combination with PLT5, or in combination with PLT7, or in combination with RKD4. RBP3 may be used in combination with PLT3, or in combination with PLT5, or in combination with PLT7, or in combination with RKD4. RBP4 may be used in combination with PLT3, or in combination with PLT5, or in combination with PLT7, or in combination with RKD4. RBP5 may be used in combination with PLT3, or in combination with PLT5, or in combination with PLT7, or in combination with RKD4. RBP6 may be used in combination with PLT3, or in combination with PLT5, or in combination with PLT7, or in combination with RKD4. RBP7 may be used in combination with PLT3, or in combination with PLT5, or in combination with PLT7, or in combination with RKD4. RBP8 may be used in combination with PLT3, or in combination with PLT5, or in combination with PLT7, or in combination with RKD4.

In one preferred embodiment of the method according to any of the embodiments described above, the at least one regeneration booster further comprises at least one PLT or RKD4, wherein the at least one PLT or RKD4 comprises an amino acid sequence selected from the group consisting of SEQ ID NOs: 28, 29, 30 and 31, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one PLT or RKD4 is encoded by a sequence selected from the group consisting of SEQ ID NOs: 16, 17, 18 and 19, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

The artificial regeneration boosters RBP1 to RBP8 or combinations thererof may also be used in combination with one or more boosters selected from BBM, WUS, WOX, GRF or LEC. RBP1 may be used in combination with BBM, or in combination with WUS, or in combination with WOX, or in combination with GRF or in combination with LEC. RBP2 may be used in combination with BBM, or in combination with WUS, or in combination with WOX, or in combination with GRF or in combination with LEC. RBP3 may be used in combination with BBM, or in combination with WUS, or in combination with WOX, or in combination with GRF or in combination with LEC. RBP4 may be used in combination with BBM, or in combination with WUS, or in combination with WOX, or in combination with GRF or in combination with LEC. RBP5 may be used in combination with BBM, or in combination with WUS, or in combination with WOX, or in combination with GRF or in combination with LEC. RBP6 may be used in combination with BBM, or in combination with WUS, or in combination with WOX, or in combination with GRF or in combination with LEC. RBP7 may be used in combination with BBM, or in combination with WUS, or in combination with WOX, or in combination with GRF or in combination with LEC. RBP8 may be used in combination with BBM, or in combination with WUS, or in combination with WOX, or in combination with GRF or in combination with LEC.

In one embodiment of the method according to any of the embodiment described above, a further regeneration booster is introduced, wherein the further regeneration booster, or the sequence encoding the same is selected from BBM, WUS, WOX, GRF, LEC, or a variant thereof or the further regeneration booster is a regeneration booster selected from RBP1 to RBP8, wherein the further regeneration booster is different from the first regeneration booster.

In one embodiment of the method according to any of the embodiments described above, the at least one regeneration booster comprises at least one RBP, wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence, and the at least one regeneration booster comprises PLT5, wherein the PLT5 comprises an amino acid sequence of SEQ ID NO: 30, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the PLT5 is encoded by a nucleic acid sequence of SEQ ID NO: 18, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

According to the various embodiments and aspects disclosed herein, it may be preferable to use a naturally occurring regeneration booster in addition to an artificial RBP according to the present invention, wherein the naturally occurring regeneration booster, e.g., BBM, WUS1/2, LEC1/2, GRF, or a PLT may be derived from a target plant to be transformed, or from a closely related species. For monocot plant modifications, for example, a booster protein with monocot origin (e.g., from Zea mays (Zm)) may be preferred, whereas for dicot plant modifications, a booster protein with dicot origin (e.g., originating from Arabidopsis thaliana (At), or Brassica napus (Bn)) may be preferred. The relevant booster sequences can be easily identified by sequence searches within the published genome data. Notably, regeneration boosters from one plant species may show a certain cross-species applicability so that, for example, a wheat-derived booster gene may be used in Zea mays, and vice versa, or a Arabidopsis- or Brachypodium-derived booster gene may be used in Helianthus, and vice versa. A PLT, WUS, WOX, BBM, LEC, RKD4, or GRF sequence as used herein, or a protein with a comparable regeneration booster function, may thus be derived from any plant species harbouring a corresponding gene encoding the respective booster in its genome.

The use of at least one regeneration booster in an artificial and controlled context according to the methods disclosed herein thus has the effect of promoting plant cell proliferation. This effect is highly favourable for any kind of plant genome modification, as it promotes cell regeneration after introducing any plasmid or chemical into the at least one plant cell via transformation and/or transfection, as these interventions necessarily always cause stress to a plant cell.

Additionally, or alternatively, the at least one regeneration booster according to the methods disclosed herein may have a specific effect in enhancing plant genome editing efficiency. In particular, this kind of intervention caused by at least one site-specific nuclease, nickase or a variant thereof, causes a certain repair and stress response in a plant. The presence of at least one regeneration booster can thus also improve the efficiency of genome or gene editing by increasing the regeneration rate of a plant cell after a modification of the plant genome.

In one embodiment, at least one regeneration booster, or a sequence encoding the same, or a regeneration booster chemical, can be provided simultaneously with other tools to be inserted, namely the at least one genome modification system, preferably the genome editing system to reduce the number of transformation/transfection acts potentially stressful for a cell. For certain cells sensitive to transformation/transfection, regeneration booster chemicals may thus represent a suitable option, which may be provided before, simultaneously with, or soon after transforming/transfecting further genome or gene editing tools to reduce the cellular stress and to increase transformation and/or editing efficiency by stabilizing a cell and thus by reducing potentially harmful cellular stress responses.

In another embodiment, the at least one genome modification system, preferably the genome editing system and the at least one regeneration booster, or the sequence encoding the same, may be provided subsequently or sequentially. By separating the introduction steps, the editing construct DNA integration of the site-directed nuclease, nickase or an inactivated nuclease encoding sequence can be avoided, where transient outcomes are of interest.

In certain embodiments, it is favourable that the at least one regeneration booster is active in a cell before further tools are introduced to put the cell into a state of low cellular stress before performing genome or gene editing.

For any simultaneous or subsequent introduction of at least one regeneration booster, the regeneration booster and the optional further genome modification or genome editing system should be active, i.e., present in the active protein and/or RNA stage, in one and the same cell to be modified, preferably in the nucleus of the cell, or in an organelle comprising genomic DNA to be modified.

In one embodiment of the methods according to any of the embodiments described above, at least one epigenetically regulating chemical is introduced in step ii) and the at least one epigenetically regulating chemical is a histone deacetylase inhibitor (HDACI), in particular trichostatin A (TSA) or a TSA like chemical.

In one embodiment of the method according to any of the embodiments described above, the at least one genome modification system, preferably the at least one genome editing system, and the at least one regeneration booster, or the sequences encoding the same and/or the at least one epigenetically regulating chemical, are introduced into the cell by transformation or transfection mediated by biolistic bombardment, Agrobacterium-mediated transformation, micro- or nanoparticle delivery, or by chemical transfection, or a combination thereof, preferably wherein the at least one genome modification system, preferably the at least one genome editing system, and the at least one regeneration booster and/or the at least one epigenetically regulating chemical are introduced by biolistic bombardment, preferably wherein the biolistic bombardment comprises a step of osmotic treatment before and/or after bombardment.

Particle or biolistic bombardment may be a preferred strategy according to the methods disclosed herein, as it allows the direct and targeted introduction of exogenous nucleic acid and/or amino acid material in a precise manner not relying on the biological spread and expression of biological transformation tools, including Agrobacterium.

Particle bombardment or biolistic bombardment refers to a physical delivery method for transferring a coated microparticle or nanoparticle comprising a construct of interest into a target cell or tissue. For use in the present invention, constructs of interest comprise genome modification components and at least one regeneration booster. The transformation via particle bombardment uses a microprojectile of metal covered with the construct(s) of interest, which is then shot onto the target cells using an equipment known as “gene gun” (Sandford et al. 1987) at high velocity fast enough (~1500 km/h) to penetrate the cell wall of a target tissue, but not harsh enough to cause cell death. The coated components on the at least one microprojectile are released into the cell after bombardment. The acceleration of microprojectiles is accomplished by a high voltage electrical discharge or compressed gas (helium). Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

The microparticle consists of a non-toxic, non-reactive material. Preferably, the microparticle comprises a metal such as gold or tungsten. The coating with genome modification and boost components can comprise one or more coating layers. For example, a microparticle may contain a first coating layer comprising genome modification component and a second coating layer comprising the regeneration booster compound. The regeneration boosters can be co-delivered with the genome modification components via microcarriers comprising gold particles having a size in a range of 0.4-1.6 micron (μm), preferably 0.4-1.0 μm. Regeneration boosters and genome modification components can be delivered into target cells for example using a Bio-Rad PDS-1000/He particle gun or handheld Helios gene gun system. More than one chemical or booster construct can be co-delivered with genome modification components into target cells simultaneously. In an exemplary process, 10 ng-10 μg of DNA, preferably 50-1000 ng of DNA, coated onto 10-1000 μg of gold particles, preferably 50-500 μg, are used per one bombardment (shot). When a PDS-1000/He particle gun system used, the bombardment rupture pressures are from 450 psi to 2200 psi, preferred from 450-1,500 psi, more preferably 450-1,100 psi. Up to 10 bombardments (shots), preferred 1-4 shots, per one sample plate can be used for the delivery of foreign molecules into plant cells.

According to a preferred embodiment of the method described above, optionally at least one epigenetically regulating chemical can be introduced into at least one plant cell via in vitro pre-treatment of plant materials in a medium containing at least one epigenetically regulating chemical, e.g., TSA. Thus, the method for genetic modification in a plant cell may further comprise a step of pretreatment of the plant cell to be used in step a), said pretreatment comprising culturing the plant cells or plant material comprising same in a medium containing at least an epigenetically regulating chemical or an active derivative thereof, in particular a histone deacetylase inhibitor (HDACI). Exemplary, as for the histone deacetylase inhibitor TSA, the duration of the TSA pre-treatment is from 10 minutes to 2 days, preferred 2.0 to 24 hours. TSA concentration for a pre-treatment is 1.0 nM to 10 mM, preferred 10 nM to 500 nM. Hereafter the treated plant materials are transferred to TSA-free medium and used for TSA co-introduction with the genetic modification components immediately as a prolonged TSA pre-treatment may cause non-selective enhancement of cell regeneration, which may increase difficulty in retrieving the modified cells.

After the optional pretreatment step, the treated plant cells are taken from the medium and used for co-introduction step by microprojectile bombardment.

### Preferred is a Co-Introduction of Epigenetically Regulating Chemical Via Microprojectile Bombardment.

In this context, the present invention provides a microparticle coated with
(i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same;
(ii) at least one epigenetically regulating chemical, preferably a histone deacetylase inhibitor (HDACI), in particularly trichostatin A (TSA) or one TSA like chemical, and
(iii) optionally, at least one regeneration booster, or a sequence encoding the same.

Preferably, the epigenetically regulating chemical and the regeneration booster, are transiently present, transiently active or transiently expressed in at least one transformed cell.

The microparticle consists of a non-toxic, non-reactive material. Preferably, the microparticle comprises a metal such as gold or tungsten. The size of the microparticle may be in a range of 0.4-1.6 micron (μm), preferably 0.4-1.0 μm. The genome modification materials are DNA, RNA, protein, or ribonucleoprotein (RNP). Plant materials are any or any part of any tissues, organs or cells, e.g., shoots, stems, leaves roots, embryos, calluses, and others. The amounts of TSA used for a bombardment with 100 μg of gold particles (approximately, 4.0-5.0×10⁷ 0.6 μm gold particles) are in range of 0.01 ng to 500 ng, preferred 0.1-50 ng. The TSA and genome modification materials are delivered into target cells using a Bio-Rad PDS-1000/He particle gun or handheld Helios gene gun system.

The coating with components (i) and (ii) can comprise one or more coating layers. For example, a microparticle may contain a first coating layer comprising genome modification components (i) and a second coating layer comprising components (ii) and/or (iii). Alternatively, a microparticle may contain a coating layer comprising genome engineering components (i) and at least one of components (ii) and (iii).

The genome modification, e.g., genome editing efficiency can be analyzed for instance by next Taqman ddPCR and/or generation sequencing (NGS).

According to another aspect of the present invention, the genetically modified plant cells can be regenerated into a whole (fertile) plant. Thus, in a preferred aspect of the invention, the genetic modification of a plant cell is followed by a step of regenerating a plant. Accordingly, the present invention provides a method for producing a genetically modified plant comprising the steps:
a) genetically modifying a plant cell according to the above method for genetic modification in a plant cell, and
b) regenerating a plant from the modified plant cell of step a),

In one embodiment of the method according to any of the embodiments described above, at least one site-directed nuclease, nickase or an inactivated nuclease, or a sequence encoding the same, is introduced and is selected from the group consisting of a CRISPR/Cas system, preferably from a CRISPR/MAD7 system, a CRISPR/Cfp1 system, a CRISPR/MAD2 system, a CRISPR/Cas9 system, a CRISPR/CasX system, a CRISPR/CasY system, a CRISPR/Cas13 system, or a CRISPR/Csm system, a zinc finger nuclease system, a transcription activator-like nuclease system, or a meganuclease system, or any combination, variant, or catalytically active fragment thereof.

In another embodiment of the method according to any of the embodiments described above, at least one genome editing system is introduced, wherein the at least one genome editing system further comprises at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACF1/ASE deaminase, an ADAT family deaminase, an ADAR2 deaminase, or a PmCDA1 deaminase, a TadA derived deaminase, and/or a transposon, or a sequence encoding the aforementioned at least one enzyme, or any combination, variant, or catalytically active fragment thereof.

A variety of suitable genome editing systems that can be employed according to the methods of the present invention, is available to the skilled person and can be easily adapted for use in the methods used herein.

In embodiments, wherein the site-directed nuclease or variant thereof is a nucleic acid-guided site-directed nuclease, the at least one genome editing system additionally includes at least one guide molecule, or a sequence encoding the same. The “guide molecule” or “guide nucleic acid sequence” (usually called and abbreviated as guide RNA, crRNA, crRNA+tracrRNA, gRNA, sgRNA, depending on the corresponding CRISPR system representing a prototypic nucleic acid-guided site-directed nuclease system), which recognizes a target sequence to be cut by the nuclease. The at least one “guide nucleic acid sequence” or “guide molecule” comprises a “scaffold region” and a “target region”. The “scaffold region” is a sequence, to which the nucleic acid guided nuclease binds to form a targetable nuclease complex. The scaffold region may comprise direct repeats, which are recognized and processed by the nucleic acid guided nuclease to provide mature crRNA. A pegRNAs may comprise a further region within the guide molecule, the so-called “primer-binding site”. The “target region” defines the complementarity to the target site, which is intended to be cleaved. A crRNA as used herein may thus be used interchangeably herein with the term guide RNA in case it unifies the effects of meanwhile well-established CRISPR nuclease guide RNA functionalities. Certain CRISPR nucleases, e.g., Cas9, may be used by providing two individual guide nucleic acid sequences in the form of a tracrRNA and a crRNA, which may be provided separately, or linked via covalent or non-covalent bonds/interactions. The guide RNA may also be a pegRNA of a Prime Editing system as further disclosed below. The at least one guide molecule may be provided in the form of one coherent molecule, or the sequence encoding the same, or in the form of two individual molecules, e.g., crRNA and tracr RNA, or the sequences encoding the same.

In certain embodiments, the genome editing system may be a base editor (BE) system.

In yet another embodiment, the genome editing system may be a Prime Editing system.

Any nucleic acid sequence comprised by, or encoding a genome modification or genome editing system disclosed herein, or a regeneration booster sequence, may be “codon optimized” for the codon usage of a plant target cell of interest. This means that the sequence is adapted to the preferred codon usage in the organism that it is to be expressed in, i.e. a “target cell of interest”, which may have its origin in different target plants (wheat, maize, sunflower, sugar beet, for example) so that a different codon optimization may be preferable, even though the encoded effector on protein level may be the same. If a nucleic acid sequence is expressed in a heterologous system, codon optimization increases the translation efficiency significantly.

In certain embodiments according to the methods as disclosed herein, it may be preferable to achieve homology-directed repair (HDR)-mediated genome editing instead of non-homologous end-joining (NHEJ). In certain embodiments according to the various aspects and methods disclosed herein, wherein at least one genome editing system is introduced, the at least one genome editing system comprises at least one repair template (or donor), and the at least one repair template comprises or encodes a double- and/or single-stranded nucleic acid sequence.

In a further embodiment of the genome editing system according to any of the embodiments described above, the system may thus additionally comprise at least one repair template, or a sequence encoding the same. A “repair template”, “repair nucleic acid molecule”, or “donor (template)” refers to a template exogenously provided to guide the cellular repair process so that the results of the repair are error-free and predictable. In the absence of a template sequence for assisting a targeted homologous recombination mechanism (HDR), the cell typically attempts to repair a genomic DNA break via the error-prone process of non-homologous end-joining (NHEJ).

In one embodiment, the at least one repair template may comprise or encode a double- and/or single-stranded sequence.

In another embodiment, the at least one repair template may comprise symmetric or asymmetric homology arms.

In another embodiment, the at least one repair template may comprise at least one chemically modified base and/or backbone, including a phosphothioate modified backbone, or a fluorescent marker attached to a nucleic acid of the repair template and the like.

In one embodiment, a genome modification or editing system according to any of the embodiments described above, the at least one site-directed nuclease, nickase or an inactivated nuclease, or a sequence encoding the same, and/or optionally the at least one guide nucleic acid, or the sequence encoding the same, and/or optionally the at least one repair template, or the sequence encoding the same, are provided simultaneously, or one after another.

In certain embodiments, the at least one genome editing system, and optionally the at least one repair template, or the respective sequences encoding the same, are introduced transiently or stably, or as a combination thereof. Whereas the stable integration of at least one genome editing system, in particular the site-directed nuclease or variant thereof, but not necessarily including at least one guide RNA, may allow a stable expression of this effector, the methods as disclosed herein can be performed in a full transient way. This implies that the tools as such are not integrated into the genome of a cell to be modified, unless at least one repair template is used. This transient approach may be preferably for a highly controllable gene editing event.

The methods of the present invention may be applied to monocotyledonous or a dicotyledonous plant as described in more detail below.

The generation and use of haploids is one of the most powerful biotechnological means to improve cultivated plants. The advantage of haploids for breeders is that homozygosity can be achieved already in the first generation after dihaploidization, creating doubled haploid plants, without the need of laborious backcrossing steps to obtain a high degree of homozygosity. Furthermore, the value of haploids in plant research and breeding lies in the fact that the founder cells of doubled haploids are products of meiosis, so that resultant populations constitute pools of diverse recombinant and at the same time genetically fixed individuals. The generation of doubled haploids thus provides not only perfectly useful genetic variability to select from with regard to crop improvement, but is also a valuable means to produce mapping populations, recombinant inbreds as well as instantly homozygous mutants and transgenic lines.

Haploid plants can be obtained by interspecific crosses, in which one parental genome is eliminated after fertilization. It was shown that genome elimination after fertilization could be induced by modifying a centromere protein, the centromere-specific histone CENH3 in Arabidopsis thaliana (Ravi and Chan, Haploid plants produced by centromere-mediated genome elimination, Nature, Vol. 464, 2010, 615-619). With the modified haploid inducer lines, haploidization occurred in the progeny when a haploid inducer plant was crossed with a wild type plant. Interestingly, the haploid inducer line was stable upon selfing, suggesting that a competition between modified and wild type centromere in the developing hybrid embryo results in centromere inactivation of the inducer parent and consequently in uniparental chromosome elimination.

In one aspect, there is provided a method of producing a haploid or doubled haploid plant cell, tissue, organ, plant, or seed.

In one embodiment, the methods of the present invention thus comprise the generation of at least one haploid cell, tissue or organ having activity of a haploid inducer, preferably wherein the haploid cell, tissue or organ comprises a callus tissue, male gametophyte or microspore. In this embodiment, the methods as disclosed herein may comprise the introduction of a nucleotide or amino acid sequence encoding or being a sequence allowing the generation of a haploid inducer cell, for example a sequence encoding a KINETOCHORE NULL2 (KNL2) protein comprising a SANTA domain, wherein the nucleotide sequence comprises at least one mutation causing in the SANTA domain an alteration of the amino acid sequence of the KNL2 protein and said alteration confers the activity of a haploid inducer (as disclosed in EP 3 159 413 A1) in a method for plant genome modification, preferably for the targeted modification of at least one genomic target sequence, for obtaining a modification of at least one plant cell. In this embodiment, the at least one genome modification system does not comprise a genome editing system, but the sequence allowing the generation of a haploid inducer line, which is introduced into a plant cell to be modified stably or transiently, in a constitutive or inducible manner.

In another embodiment, the modified cell according to the methods of the present invention is a haploid cell, wherein the haploid cell is generated by introducing a genome editing system into at least one cell to be modified, wherein the genome editing system is capable of introducing at least one mutation into the genomic target sequence of interest resulting in a cell having haploid inducer activity.

In yet a further aspect, there is provided a method of producing a haploid or doubled haploid plant cell, tissue, organ, plant, or seed, wherein the method comprises providing at least one regeneration booster, or a specific combination of regeneration boosters, or the sequence(s) encoding the same, to at least one cell to be modified, wherein the at least one cell is preferably a haploid cell, for example, a gametophyte or microspore. These inherently haploid cells of plants produced during the reproduction cycle have the intrinsic characteristic of being very inert to any kind of chromosome doubling and transformation. The methods as disclosed herein can thus be favourably used to introduce or apply at least one regeneration booster, or a sequence encoding the same for promoting the regenerative capacity of a haploid plant cell to increase the capacity of the haploid cell for a conversion during chromosome doubling, as the doubled haploid material is of particular interest for breeding and ultimately cultivating plants. The methods as disclosed herein thus overcome the difficulties in handling haploid plants cells and tissues, including callus tissue, as the frequency of induced and/or spontaneous chromosome doubling can be increased by providing at least one booster sequence, or preferably a specific combination of booster sequences, as disclosed herein.

Various methods for doubling chromosomes in plant biotechnology are available to the skilled person for various cultivars. In one embodiment, chromosome doubling can be achieved by using colchicine treatment. Other chemicals for chromosome doubling, are available for use according to the methods disclosed herein, wherein these chemicals may be selected from antimicrotubule herbicides, including amiprophosmethyl (APM), pronamide, oryzalin, and trifluralin, which are all known for their chromosome doubling activity.

In one aspect, the present invention also relates to the use of a regeneration booster or a combination of regeneration boosters, preferably a regeneration booster or combination of regeneration boosters as described in any of the embodiments above, in a method for targeted plant genome modification, preferably in a method according to any of the embodiments described above, to
(a) promote plant cell proliferation and/or assist in the targeted modification and/or provide a positive selection for at least one transiently transformed cell for genome modification and/or for regeneration into at least one modified plant, and/or
(b) suppress plant cell differentiation if the cell is stably transformed and provide a negative selection for at least one stably transformed cell for regeneration into one transgenic plant, and/or
(c) provide a dual selection according to (a) and (b) for at least one transformed cell.

As mentioned above, the regeneration booster(s) increase genome editing efficiency and at the same time promote proliferation of transiently transformed cells while suppressing plant cell differentiation of stably transformed cells. This dual selection advantageously allows to regenerate a modified plant, which does not pass on the genome editing tools to its progeny but merely carries the desired modification.

In one aspect the present invention relates to a plant cell, tissue, organ, plant or seed obtainable by or obtained by a method according to any of the embodiments described above.

In one embodiment of the plant cell, tissue, organ, plant or seed described above, the plant cell, tissue, organ, plant or seed originates from a monocotyledonous or a dicotyledonous plant selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant cell, tissue, organ, plant or seed originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

In a further aspect, the present invention relates to an expression construct assembly, comprising
(i) at least one vector encoding at least one genome modification system which comprises at least one gene of interest, preferably an exogenous gene of interest, and/or which is preferably at least one genome editing system comprising at least one site-directed nuclease, nickase or an inactivated nuclease of a genome editing system, preferably wherein the genome editing system is as defined above, and
(ii) at least one vector encoding at least one regeneration booster, preferably wherein the regeneration booster or boosters is/are as defined in any of the embodiments described above, and
(iii) optionally, when the at least one site-directed nuclease, nickase or an inactivated nuclease of a genome editing system is a nucleic acid guided nuclease: at least one vector encoding at least one guide molecule guiding the at least one nucleic acid guided nuclease, nickase or an inactivated nuclease to the at least one genomic target site of interest; and
(iv) optionally: at least one vector encoding at least one repair template;
wherein (i), (ii), (iii), and/or (iv) are encoded on the same, or on different vectors.

In one embodiment, the expression construct assembly may further comprise a vector encoding at least one marker, preferably wherein the marker is introduced in a transient manner, see, for example, SEQ ID NO: 55.

In one embodiment, the expression construct assembly comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

Notably different components of a genome modification or editing system and/or a regeneration booster sequence and/or a guide molecule and/or a repair template present on the same vector of an expression vector assembly may comprise or encode more than one regulatory sequence individually controlling transcription and/or translation.

In one embodiment of the expression construct assembly described above, the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In another embodiment of the expression construct assembly described above, the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In a further embodiment of the expression construct assembly described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the expression construct assembly according to any of the embodiments described above, the construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In addition, the expression construct assembly may comprise at least one terminator, which mediates transcriptional termination at the end of the expression construct or the components thereof and release of the transcript from the transcriptional complex.

In one embodiment of the expression construct assembly according to any of the embodiments described above, the regulatory sequence may comprise or encode at least one terminator selected from the group consisting of nosT, a double 35S terminator, a ZmEf1 terminator, an AtSac66 terminator, an octopine synthase (ocs) terminator, or a pAG7 terminator, or a combination thereof. A variety of further suitable promoter and/or terminator sequences for use in expression constructs for different plant cells are well known to the skilled person in the relevant field.

Exemplary elements of an expression vector assembly of the present invention, which may be individually combined, may comprise a suitable vector backbone, wherein a variety of suitable vectors are available in plant biotechnology, an expression cassette, i.e., a cassette encoding a sequence of an effector, for example, at least one regeneration booster as disclosed herein, for example, according to any one of SEQ ID NOs: 4 to 15; an expression construct, i.e., a construct including an expression cassette and at least one further vector element, for example, as represented in any one of SEQ ID NOs: 40 to 51; a vector or expression construct comprising or encoding at least one site-directed nuclease, for example, as represented in any one of SEQ ID NOs: 52 or 54; a suitable vector encoding a guide molecule, in case a nucleic acid-guided site directed nuclease is used, specific for a genomic target sequence of interest, for example, a sequence according to SEQ ID NO: 53, wherein the respective guide molecule is compatible with the cognate nucleic acid-guided site directed nuclease, or variant thereof, wherein the guide molecule comprised or encoded can be easily replaced by another guide molecule targeting a different genomic target site of interest; a vector encoding at least one repair template sequence of interest; and/or a vector or expression construct comprising or encoding at least one expressible marker gene, preferably a marker gene, which can be easily detected macroscopically, or microscopically, like a fluorescent marker gene as encoded by, for example, SEQ ID NO: 55. A variety of suitable fluorescent marker proteins and fluorophores applicable over the whole spectrum, i.e., for all fluorescent channels of interest, for use in plant biotechnology for visualization of metabolites in different compartments are available to the skilled person, which may be used according to the present invention. Examples are GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum), an orange, a red or far-red fluorescent protein (e.g., tdTomato (tdT), or DsRed), and any of a variety of fluorescent and coloured proteins may be used depending on the target tissue or cell, or a compartment thereof, to be excited and/or visualized at a desired wavelength.

All elements of the expression vector assembly can be individually combined. Further, the elements can be expressed in a stable or transient manner, wherein a transient introduction may be preferably. In certain embodiments, individual elements may not be provided as part of a yet to be expressed (transcribed and/or translated) expression vector, but they may be directly transfected in the active state, simultaneously or subsequently, and can form the expression vector assembly within one and the same cell of interest to be modified. For example, it may be reasonable to first transform part of the expression vector assembly encoding a site-directed nuclease, which takes some time until the construct is expressed, wherein the cognate guide molecule is then transfected in its active RNA stage and/or at least one repair template is then transfected in its active DNA stage in a separate and subsequent introduction step to be rapidly available. The at least one regeneration booster sequence and/or the at least one genome modification or editing system and/or the at least one marker may also be transformed as part of one vector, as part of different vectors, simultaneously, or subsequently. The use of too many individual introduction steps should be avoided, and several components can be combined in one vector of the expression vector assembly, to reduce cellular stress during transformation/transfection. In certain embodiments, the individual provision of elements of the at least one regeneration booster sequence and/or the at least one genome modification or editing system and/or the at least one marker and/or the at least one guide molecule and/or the at least one repair template on several vectors and in several introduction steps may be preferable in case of complex modifications relying on all elements so that all elements are functionally expressed and/or present in a cell to be active in a concerted manner.

In one aspect, the present invention also relates to a plant cell, tissue, organ, plant or seed comprising the at least one regeneration booster as described above or the expression construct assembly as described above.

In another aspect, the present invention relates to a method for selecting a modified plant cell, plant tissue, organ, plant or seed, wherein the method comprises the steps (a) to (c) as defined in the method for plant genome modification according to any of the embodiments described above and further comprises the step:
(d1) screening for at least one plant cell carrying a targeted modification, or
(d2) screening for at least one plant tissue, organ, plant or seed regenerated from at least one modified plant cell in the T0 or T1 generation carrying a desired targeted modification.

The method for selecting a modified plant cell, plant tissue, organ, plant or seed may be without a conventional selection step. A conventional selection step refers to any processes to select and purify the transformed cells from wild-type cells by using an integrated selection marker, e.g. antibiotic (e.g. kanamycin, hygromycin), or herbicide (e.g. phosphinothricin, glyphosate) resistance gene. Without a conventional selection, such a plant or seed may not have any of the genome modification components integrated, and thus leads to transgene-free genetic modified plants.

The positive impact of the regeneration booster on plant cell division and stem cell identity endows a selective regeneration of the transformed cells at the initiation stage of regeneration, whereas the boosting effect of a booster on stem cell identity, which negatively impacts cell differentiation, allows a negative selection for the cells with continuous activity of the booster (i.e. stably transformed cells) at the subsequent regeneration.

The regeneration boosters and combination of boosters described herein significantly improve the regeneration capabilities of plant from single cell origin. In particular, plants/plant genotypes that are considered recalcitrant to regeneration can be regenerated efficiently.

In a further aspect, the present invention also relates to a method for regenerating a plant tissue, organ or plant from at least one plant cell or a single plant cell, wherein the method comprises:
(i) Introducing into the at least one plant cell or the single plant cell at least one regeneration booster, or a sequence encoding the same, wherein the at least one regeneration booster is transiently present, transiently active or transiently expressed in the plant cell, preferably wherein the at least one regeneration booster(s) is/are as defined in any of the embodiments described above, and
(ii) regenerating a plant tissue, organ or plant from at least one plant cell or the single plant cell.

In one embodiment of the method for regenerating a plant tissue, organ or plant from a single plant cell, the at least one regeneration booster comprises at least one RBP, wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

The artificial regeneration boosters RBP1 to RBP8 may be used in any combination with each other or and/or in combination with other boosters as disclosed above in the context of the method for plant genome modification. Preferably, RBP1 to RBP8 or any combination thereof are combined with one of a PLT or RKD booster.

In one preferred embodiment of the method for regenerating a plant tissue, organ or plant from a single plant cell according to any of the embodiments described above, the at least one regeneration booster further comprises at least one PLT or RKD4, wherein the at least one PLT or RKD4 comprises an amino acid sequence selected from the group consisting of SEQ ID NOs: 28, 29, 30 and 31, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one PLT or RKD4 is encoded by a sequence selected from the group consisting of SEQ ID NOs: 16, 17, 18 and 19, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

The artificial regeneration boosters RBP1 to RBP8 or combinations thereof may also be used in combination with one or more boosters selected from BBM, WUS, WOX, GRF or LEC.

In one embodiment of the method for regenerating a plant tissue, organ or plant from a single plant cell according to any of the embodiment described above, a further regeneration booster is introduced, wherein the further regeneration booster, or the sequence encoding the same is selected from BBM, WUS, WOX, GRF, LEC, or a variant thereof or the further regeneration booster is a regeneration booster selected from RBP1 to RBP8, wherein the further regeneration booster is different from the first regeneration booster.

In one embodiment of the method for regenerating a plant tissue, organ or plant from a single plant cell according to any of the embodiments described above, the at least one regeneration booster comprises at least one RBP, wherein the at least one RBP comprises an amino acid sequence selected from the group consisting of SEQ ID NOs: 32, 33, 34, 35, 36, 37, 38 and 39, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one RBP is encoded by a sequence selected from the group consisting of SEQ ID NOs: 20, 21, 22, 23, 24, 25, 26 and 27, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence, and the at least one regeneration booster comprises PLT5, wherein the PLT5 comprises an amino acid sequence of SEQ ID NO: 30, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the PLT5 is encoded by a nucleic acid sequence of SEQ ID NO: 18, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

As already mentioned above, the generation of haploids is highly desirable for plant breeders as homozygosity can already be obtained in the first generation. However, it is often difficult to regenerate a plant from the haploid cells, in particular from microspores. The booster and booster combinations disclosed herein offer a significant improvement in the regeneration capabilities of haploid plants.

In one embodiment of the method for regenerating a plant tissue, organ or plant described above, the at least one plant cell is an immature embryo cell or meristematic cell, in particular a cell of a zygotic or somatic embryo or meristem or the single plant cell is a diploid cell or a haploid cell, in particular a haploid microspore.

The present invention is further illustrated by the following non-limiting examples.

### Examples

Transformation and transgene expression may be monitored by use of a report gene, such as a green fluorescent gene (GEP) in construct pGEP837 (FIG. 1), which encodes a bright monomeric green fluorescent protein with excitation maximum at 506 nm and emission maximum at 517 nm. Another report gene used in the present invention is the red fluorescent report gene tdTomato in construct pGEP359 (FIG. 13), which encodes an exceptionally bright red fluorescent protein with excitation maximum at 554 nm and emission maximum at 581 nm. The genome editing efficiency can be analyzed for instance by marker capillary electrophoresis analysis, qPCR, or Digital PCR. Site-specific modification was further conformed by next generation sequencing (NGS), Sanger sequencing, or Sanger sequencing trace decomposition analysis.

Unless stated otherwise in the examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Cray, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: A Toolbox of Different Regeneration Boosters Stimulate Cell Proliferation and Regeneration (FIGS. 1 to 11)

### Gene Cloning and Construct Generation

Maize ZmPLT5 gene was cloned by RT-PCR using total RNA isolated from maize A188 immature embryos. KWS_RBP genes (KWS_RBP2-8) were maize-codon optimized from its protein sequence, and synthesized by Integrated DNA Technologies (IDT, San Diego, CA, USA). The boost gene fragments are cloned into expression vector pABM-BdEF1 at the cloning site of BamHI and HindIII, and expressing under the control of BdEF1 promoter (BdFE1) and nos terminator (nos-T). The BdFE1 promoter is a strong constitutive promoter from Brachypodium. The sequencing-confirmed construct maps are shown in FIGS. 3 to 10.

Preparation of Maize Immature Embryo for Bombardment 9-12 days post pollination, maize ears with immature embryos size 0.5 to 2.5 mm, preferred 0.8-1.5 mm were harvested. The ears were sterilized with 70% ethanol for 10-15 minutes. After a brief air-dry in a laminar hood, remove top ~⅓ of the kernels from the ears with a shark scalpel, and pull the immature embryos out of the kernels carefully with a spatula. The fresh isolated embryos were placed onto the bombardment target area in an osmotic medium plate (N6OSM medium) with scutellum-side up. Wrap the plates with parafilm and incubated them at 25° C. in dark for 4-20 hours (preferred 4 hours) before bombardment.

### Particle Bombardment

Construct pGEP837 (SEQ ID NO: 52) contains the expression cassettes of CRISPR nuclease MAD7 and green fluorescence report gene GEP (FIG. 1), while pGEP842 (SEQ ID NO: 53) harbors the expression cassette of CRISPR crRNA m7GEP1, which targets to maize endogenous HMG13 gene (FIG. 2). Per bombardment, 100 ng of plasmid pGEP837 and 150 ng of plasmid pGEP842 were co-coated with 100 ng of different boost constructs onto 100 μg of 0.4 μm gold particles using calcium-spermidine method. Three bombardments (shots) per sample plate were performed. For 10 shots, 1 mg of gold particle in 50% (v/v) glycerol (100 μg of gold particles per shot) in a total volume of 100 microliter (μl) was pipetted into a clear low-retention microcentrifuge tube. Sonicate for 15 seconds to suspend the gold particles. While vortex at a low speed, add the following in order to each 100 μl of gold particles:
Up to 10 μl of DNA (1.0-10.0 μg total DNA of pre-mixed, 100-1000 ng per each shot)
100 μl of 2.5 M CaCl₂ (pre-cold on ice)
40 μl of 0.1 M cold spermidine

Close the lid and vortex the tube for 2-30 minutes at 0-10° C., and spin down the DNA-coated gold particles. After washing in 500 μl of 100% ethanol for two times, the pellet was resuspended in 120 μl of 100% ethanol. While vortexing at a low speed, pipet 10 μl of co-coated gold particles with a wide open 20 μl tip from the tube onto the center of the macrocarrier evenly since the particles tend to form clumps at this point, get the gold particles onto the macrocarriers as soon as possible. Air dry. Bombardment was conducted using a Bio-Rad PDS-1000/He particle gun. The bombardment conditions are: 28-30 mm/Hg vacuum, 450 or 650 psi rupture disc, 6 mm gap distance, the specimen platform is in the second position from the bottom in the chamber at a distance of 60 mm, three shots per sample plate (for maize immature embryos). Keep the coated gold particles at low temperature (e.g. 0-10° C.) as much as possible. The prepared macro-carriers with coated gold particles should be bombard into plant cells within 3 hours after preparation.

### Embryogenic Callus Induction

After bombardment the embryos were remained on the osmotic medium (N6OSM) for another 16-20 hours. Transient transformation was examined using a fluorescence microscope for the fluorescence report gene expression at excitation maximum 506 nm and emission maximum 517 nm. The embryos were transferred from the osmotic medium onto embryogenic callus induction medium (e.g. N6_5Ag) in a petri dish plate (100×25 mm) with scutellum-face-up, at a density of 12-15 embryos per plate. The embryos were incubated at 27° C. in dark for the embryogenic callus induction for 7-14 days.

Four days after embryogenic callus induction the cell proliferation and embryogenesis from the scutellum surface of immature embryos were examined under a Zeiss stereo microscope. The representative results were showed in FIG. 11. Without a regeneration booster the immature embryos from the recalcitrant maize elites did not produce any regenerating structures (FIGS. 11A and I). Compared to the control without a booster, multiple embryogenic cell structures were induced from the embryos co-bombarded with different regeneration boosters (compare FIGS. 11 A to B-H, and I to J-L). The boosters significantly promote cell proliferation and embryogenic callus induction in maize elites (FIG. 11). The regeneration boosters offer a positive selection for the transformed cells in cell proliferation and regeneration.

### Media:

### N6OSM:

N6 salt, N6 vitamin, 1.0 mg/L of 2, 4-D, 100 mg/L of Caseine, 0.7 g/L of L-proline, 0.2 M Mannitol (36.4 g/L), 0.2 M sorbitol (36.4 g/L), 20 g/L sucrose, 15 g/L of Bacto-agar, pH 5.8.

### N6_5Ag:

N6 salt, N6 vitamin, 1.0 mg/L of 2, 4-D, 100 mg/L of Caseine, 2.9 g/L of L-prolin, 20 g/L sucrose, 5 g/L of glucose, 5 mg/L of AgNO3, 8 g/L of Bacto-agar, pH 5.8.

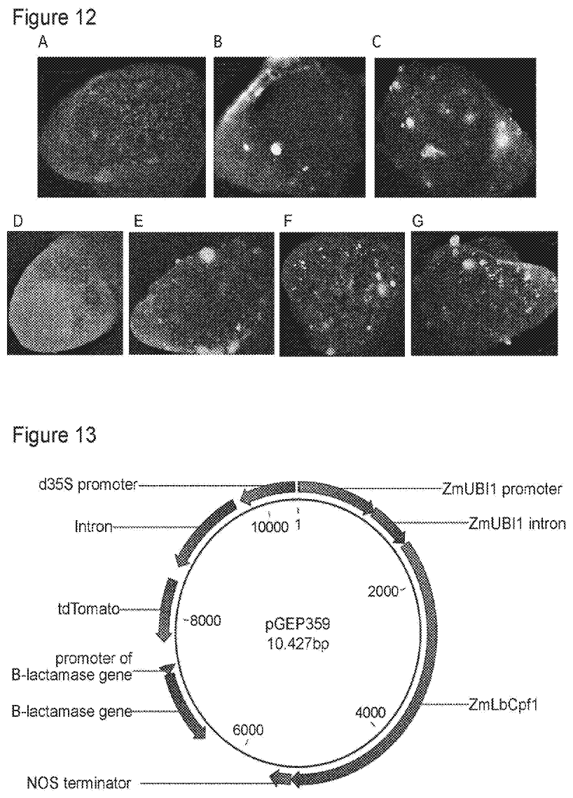

### Example 2: A Toolbox of Different Boosters Promote Rapid Genetic Transformation of Plant Cells (FIG. 12)

The expression cassette of green fluorescence report gene GEP in construct pGEP837 (SEQ ID NO: 52) was co-bombarded with plasmid pGEP842 (SEQ ID NO: 53) and different constructs encode for the expression cassette of a specific booster as indicated. For immature embryo isolation, bombardment, and embryogenic callus induction, please see the description in Example 1.

Green fluorescence report gene expression in the scutellum surface cells of the bombarded immature embryos was examined using a stereo fluorescence microscope at excitation maximum 506 nm and emission maximum 517 nm after bombardment and cultured in embryogenic callus induction for 4 days. The representative results are present in FIG. 12.

Without a booster the immature embryos from maize recalcitrant elite WA4-29814 and 4V-40214 did not produce a green fluorescent cell cluster (FIGS. 12A and D). Contrast to the control without a booster, multiple embryogenic structures with strong and uniformed green fluorescence signals appeared from the embryos co-bombarded with different regeneration boosters after bombardment and cultured for 4 days (FIGS. 12. B to C, and E to G). Strong and uniformed expression of green fluorescent report gene in regenerated tissues, e.g. embryogenic callus, indicates the integration and stable transformation of the foreign gene.

In a regular plant transformation system, it takes a few rounds of selection to identify and purify the cells with stable DNA integration to recover a homogenous transgenic plant. The selection process normally take a few weeks, e.g. 6-8 weeks for maize, depends on the speed of cell proliferation. It takes generally more than 3 month to obtain a stable transformation event in maize. The results present here reveal that boosters remarkably promote rapid stable transformation in maize elites.

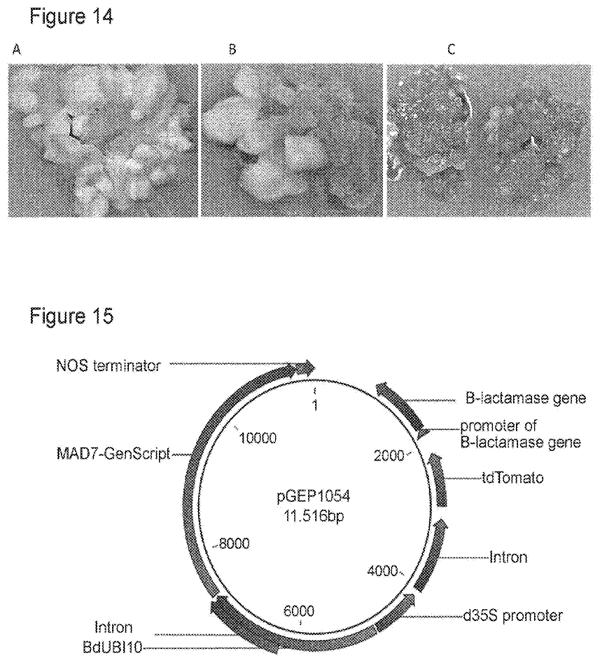

### Example 3. Continuous Expression of Boosters Negatively Impact Cell Differentiation and Plant Regeneration in Maize Recalcitrant Elite 4V-40171 (FIGS. 13 to 14)

For immature embryo isolation, osmotic treatment, bombardment, and embryogenic callus induction, please see the description in Example 1. Specifically, construct pGEP359 (SEQ ID NO: 54) that contains the expression cassette of red fluorescence report gene tDTomato (FIG. 13) was co-bombarded with the regeneration boosters of ZmPLT5 and KWS_RBP4. For each bombardment, 100 ng of plasmid pGEP359 were co-coated with 100 ng each of the two boost constructs onto 100 μg of 0.4 μm gold particles using calcium-spermidine method. Three bombardments (shots) per sample plate were performed. Embryogenic calluses were transferred onto embryo maturation medium MRM1 (please see below) after embryogenic callus induction for 12 days in N6_5Ag medium.

Strong tDTomato expression is visible by grey shades on the tissue representing red color (FIG. 14), which allow to distinguish tDTomato-positive tissues from the negative ones. tDTomate-positive calluses also indicates stable transformation of the report gene in the regenerated cells. Due to the nature of co-bombardment, the boost genes are likely co-present with tDTomato, and thus the expression of tDTomato can be used as a report for the presence of the co-bombarded boosters.

After cultured on embryo maturation medium (e.g. MRM1) for 10 days, tDTomato-negative embryogenic calluses were developed into mature embryos (FIG. 14A), while the tDTomato-positive calluses (in red color) generally remained as embryogenic calluses, but not developed into a mature embryo (FIGS. 14B and C). These results revealed a negative impact of the boosters on the further development of transformed cells in subsequent regeneration process if the booster is stably expressed and active.

The regeneration boosters stimulate cell proliferation and offer a positive selection for the initial regeneration of transformed cell. However, continuous expression of boosters negatively impact plant cell differentiation and the subsequent regeneration, and thus provide a negative selection for the stable transformed cells during plant regeneration.

Taken together, the results demonstrate that the regeneration boosters offer a “dual selection” for the transformed cells, namely positively selects transient expression delivery and negatively selects stable transgenic cells during regeneration.

### Media

### MRM1:

MS Salts, MS vitamins, 100 mg/L of myoinositol, 6% sucrose, 9 g/L of Bactoagar, pH 5.8.

### Example 4. Regeneration Boosters Enhance Genome Editing in Maize A188 Immature Embryos (Table 1, FIGS. 15 to 17, 32 and 33)

Cell environment (e.g. cells at different cycle phases) and epigenetic status and chromatin structure of cell genome influences genetic modification. It is generally believed that highly dividing cells are the best recipients for genetic modification, e.g. stable transformation. Regeneration boosters promote cell division and rapid stable transformed as demonstrated above. However, it was further assessed whether the boosters enhance genome editing as well.

For immature embryo isolation, bombardment, and embryogenic callus induction post bombardment, please see the description in Example 1.

Specifically, 100 ng of booster KWS_RBP2 construct (FIG. 4) were co-delivered with 100 ng of plasmid pGEP1054 [containing the expression cassettes of CRISPR nuclease MAD7 and report gene tDTomato (SEQ ID NO: 55, FIGS. 15)] and 300 ng of plasmid pGEP842 [containing CRISPR guide RNA m7GEP1 expression cassette, which target to maize HMG13 gene (SEQ ID NO: 53, FIG. 2) into maize A188 immature embryos by particle bombardment. tDTomato expression in the bombarded embryos was examined using a stereo fluorescence microscope at excitation 554 nm and emission 581 nm 16 hours bombardment, and results were showed in FIG. 16. The bombarded embryos were sampled at the time points as indicated (see e.g. FIG. 17). SDN-1 efficiency in the sampled embryos was analyzed by Taqman ddPCR.

The SDN-1 efficiencies are present in Table 1 and FIG. 17. Considering the time needed for gene expression of the bombarded constructs, SDN-1 efficiencies in the embryos 16 hours after bombardment are likely demonstrating the original editing activity in transformed cells without an influence from cell proliferation. The results suggest that the booster KWS_RBP2 enhances genome editing itself, not just as a result from the promoting effect on cell division.

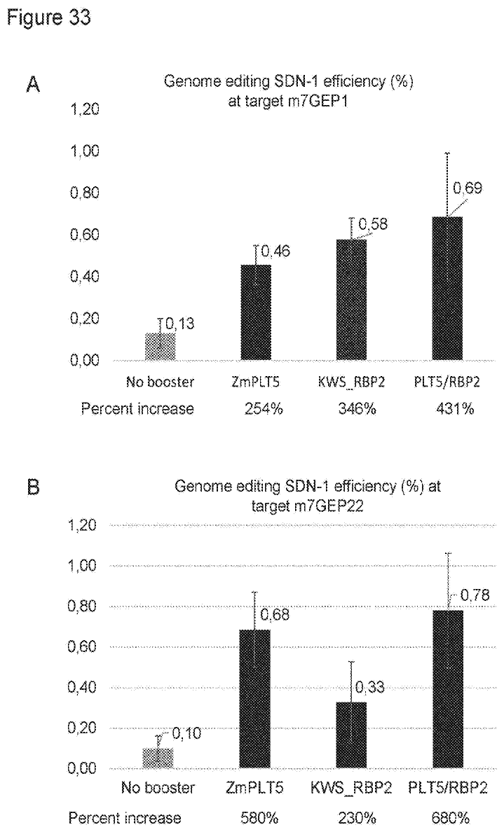

One separate experiment with a focus on booster effect on genome editing SDN-1 efficiency at two CRISPR targets 48 hours after bombardment were further conducted. 200 ng of booster ZmPLT5 (FIG. 3), or 100 ng of booster KWS_RBP2, or the combination of 200 ng ZmPLT5 and 100 ng KWS_RBP2 were co-delivered with 100 ng of plasmid pGEP1054 and 150 ng of plasmid pGEP842 or with 100 ng of pGEP1054 and 150 ng of plasmid pGEP1067 [containing CRISPR guide RNA m7GEP22 expression cassette, which target to maize HMG13 gene (SEQ ID: 56, FIG. 33)] into maize A188 immature embryos by particle bombardment. The bombarded embryos were sampled 48 hours after bombardment.

Genome editing SDN-1 efficiencies in the sampled embryos were analyzed by Taqman ddPCR and shown in FIG. 33. Compared to the control without a booster (No booster), an increase within a range of 230% to 680% in the SDN-1 efficiency at the two CRISPR target sites were observed. The best boosting effect were noted when the CRISPR editing constructs were co-introduced with the combination of the two boosters of ZmPLT5 and KWS_RBP2 (FIG. 33, PLT5/RBP2). These results further support the idea of that boosters promote genome editing SDN-1 at molecular and cell levels.

TABLE 1
SDN-1 efficiencies in the bombarded maize
A188 immature embryos sampled at a time as indicated

| | 16 HPB | 2 DPB | 3 DPB | 7 DPB |
|---|---|---|---|---|
| MAD7 only | 0 | 0.03 | 0.04 | 0.04 |
| MAD7 & sgRNA | 0.39 | 0.66 | 0.22 | 0.26 |
| MAD7, sgRNA & KWS_RBP2 | 0.50 | 1.48 | 1.16 | 2.26 |

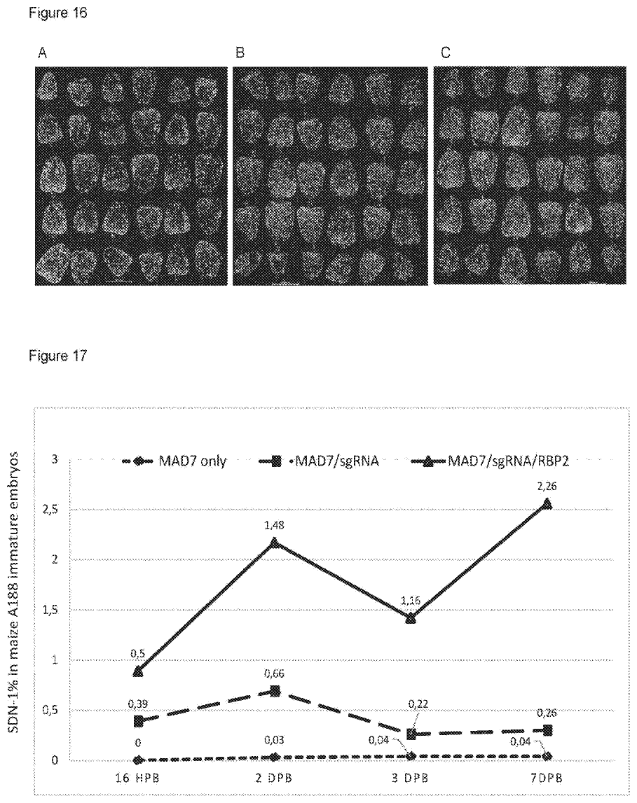

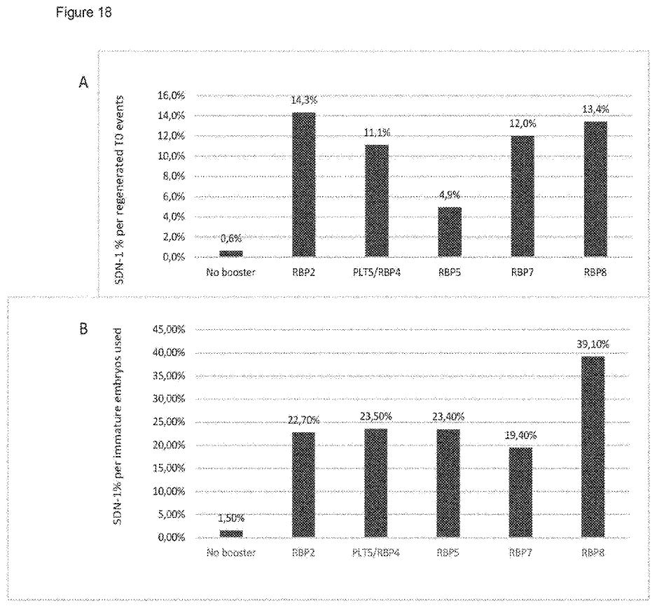

### Example 5. A Toolbox of Different Boosters Enhance Genome Editing in T₀ Plants of Maize A188 (Table 2, FIG. 18)

For immature embryo isolation, bombardment, and embryogenic callus induction post bombardment, please see the description in Example 1.

Specifically, construct pGEP837 (SEQ ID NO: 52, FIG. 1) and the crRNA construct pGEP842 (SEQ ID NO: 53, FIG. 2) were co-bombarded with the different boosters as indicated into maize A188 immature embryos by particle bombardment. For each bombardment (shot), 100 ng of plasmid pGEP837 were co-coated with 150 ng of plasmid pGEP842 and 100 ng each of different boost constructs onto 100 μg of 0.4 μm gold particles using calcium-spermidine method. Three bombardments (shots) per sample plate were performed.

After bombardment the embryos were remained on the osmotic medium (N6OSM) for another 16-20 hours. The embryos were transferred from the osmotic medium onto embryogenic callus induction medium (e.g. N6_5Ag) in a petri dish plate (100×25 mm) with scutellum-face-up, at a density of 12-15 embryos per plate. The embryos were incubated at 27° C. in dark for the embryogenic callus induction. After embryogenic callus induction in N6_5Ag medium for 10-14 days the induced calluses were transferred onto embryo maturation medium MRM1 in petro dish (100×25 mm), and cultured at 27° C. in dark for 10 days, and then at 25° C., weak light (20-50 μmol m⁻² s⁻¹) for 2-4 days. The mature somatic embryos were then transferred onto MSO medium (see below) in phytotray for plant outgrowth and further development for 5-7 days. A 5-10 mm leaf tip from each of the leaves of a T₀ plant is collected for DNA extraction. The targeted genome editing SDN-1 in the regenerated T₀ plants are screened by TaqMan real-time PCR (qPCR), and further conformed by Sanger sequencing trace decomposition analysis. The SDN-1 results are showed in Table 2 and FIG. 18.

TABLE 2
Different boosters enhance genome editing in maize A188.

| | Total | Total | Bi- | Mon- | Total | % SDN-1/ | % SDN-1/ |
|---|---|---|---|---|---|---|---|
| Boosters | IEs | Events | SDN-1 | SDN-1 | SDN-1 | Event | IE |
| No booster | 130 | 312 | 2 | 0 | 2 | 0.6% | 1.5% |
| RBP2 | 150 | 237 | 20 | 14 | 34 | 14.3% | 22.7% |
| PLT5/RBP4 | 51 | 108 | 8 | 4 | 12 | 11.1% | 23.5% |
| RBP5 | 47 | 224 | 8 | 3 | 11 | 4.9% | 23.4% |
| RBP7 | 31 | 50 | 5 | 1 | 6 | 12.0% | 19.4% |
| RBP8 | 23 | 67 | 5 | 4 | 9 | 13.4% | 39.1% |
| Avg with | 302 | 686 | 46 | 26 | 72 | 10.5% | 23.8% |
| booster | | | | | | | |

Maize A188 is a highly regenerative genotype, and has been widely used as a model for plant study. Using the rapid regeneration system present here we are able to recover genome edited T₀ plants without a conventional selection and booster at a feasible efficiency (e.g. 0.6% of SDN-1 per T0 plant or 1.5% of SDN-1 per immature embryo initially used) from maize A188. However, when a booster was co-delivered the SDN-1 efficiency was dramatically increased (e.g. >15 fold increase in average when compared to that without a booster). An average 10% of SDN-1 per regenerated T0 plant or 24% per immature embryo is achieved (Table 2; FIG. 18). These results indicate that boosters promote genome editing and offer a positive selection for plant regeneration of the transformed cells. A toolbox of different boosters for highly efficient genome editing in maize A188 was developed.

### Media:

MS0: MS Salts, MS vitamins, 2 g/L of myoinositol, 2% sucrose, 8 g/L of Bactoagar, pH 5.8

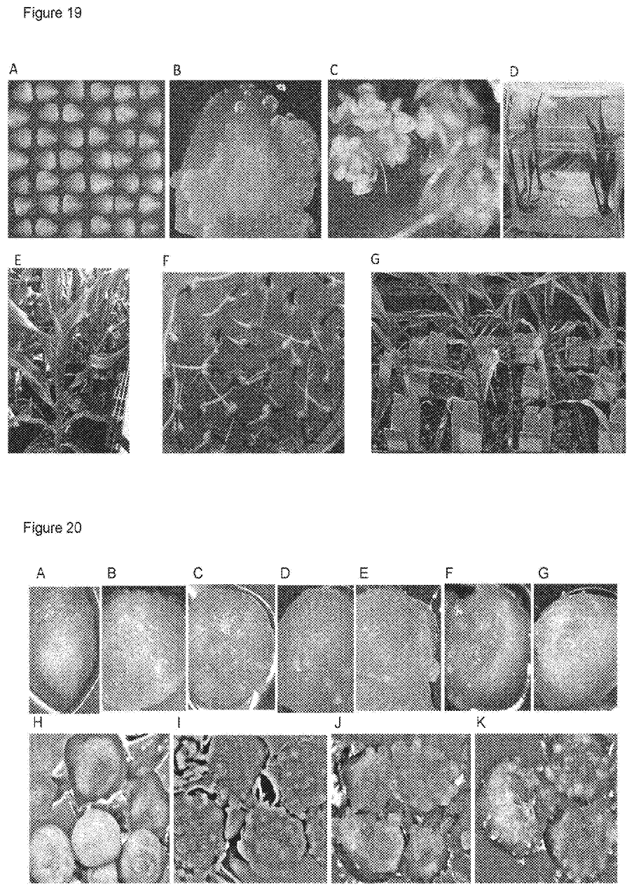

### Example 6: Using Regeneration Boosters as a “Dual Selection” for Rapid Regeneration and Genome Editing Directly in Maize Recalcitrant Elites (FIG. 19)

For immature embryo isolation, bombardment, and embryogenic callus induction post bombardment, please see the description in Example 1.

### Workflow for Rapid Genome Editing in Maize

The rapid regeneration and genome editing in maize comprising the steps:
a) Preparing plant cells as part of preferably immature embryos (IEs), either zygotic embryos or somatic embryos.
b) Delivery of genome modification/editing components via particle bombardment into the plant cells as part of preferably immature embryos (IEs) from a),
c) Culturing the plant cells from b) in the conditions allow for genome modification occur, and
d) Regenerating a plant from the modified plant cell of step c), comprising of:
   i) Plant cell proliferation and embryogenic callus induction, in which the cells from scutellum surface in immature embryos multiple and are organized into regenerative calluses. Specifically, 16-48 hours after particle bombardment, the bombarded immature embryos are transferred onto a embryogenesis callus induction medium containing relatively high concentration of auxin, e.g. 2,4-D (e.g. N6_5Ag, please also see Example 1-6) in petro dish plate (e.g. 25 100 mm), and culture at 15-30° C., dark for 1-4 weeks, preferably 1-3 weeks, most preferably 7-14 day (Note: the boosters offer a positive selection for the transiently transformed cells during this step).
   ii) Plant organ/embryo development from the cells in i): separate developing embryogenic calluses from i) into small pieces of 1-10 mm, preferably 2-7 mm in diameter, and transfer the calluses onto a plant organ/embryo development medium in petro dish (e.g. 25×100 mm) depending on different genotypes. An embryo development medium contains high concentration of sucrose (e.g. 6% of sucrose) without plant hormone (e.g. MRM1 medium), while a plant organ development medium contains a high ratio of cytokinin (e.g. 6-Benzylaminopurine, Zeatin) to auxin (e.g. NAA, or IAA), e. g. the shooting medium (see below). Seal the plate with parafilm/surgical tape and culture at 15-30° C., light (50-200 μmol m⁻² s⁻¹) for 1-4 weeks, preferably 1-3 weeks, most preferably 10-18 days [Note: the boosters offer a negative selection for the stably transformed cells during this step; a “dual selection” for the cells with the booster activities only in regeneration step (a) degreed in step (b)]. Please also see Example 6.
   iii) Plant development from the organs and embryos regenerated from ii): transfer the developing plantlets from ii) onto a plant development medium in phytotray (e.g. MSO or Rooting medium, see below), and culture at 15-30° C., light (50-200 μmol m⁻² s⁻¹) for 1-4 weeks, preferably 5-20 days. Now the regenerated T0 plants are ready for leaf sampling for molecular analysis or transfer to soil for T₁ seed production.
e) Screen for genetic modification events in the regenerated T₀ plants

A 5-20 mm leaf tip from each of the leaves of a T₀ plant is collected for DNA extraction. Genome modification, e.g. genome editing in the regenerated T₀ plants are screened for instance by TaqMan real-time PCR (qPCR), marker capillary electrophoresis analysis, and TaqMan Digital Droplet PCR. Site-specific modification is further conformed by next generation sequencing (NGS), Sanger sequencing, or Sanger sequencing trace decomposition analysis.
f) Grow the modified T₀ plants for T₁ seed production.

After the molecular screening and confirmation the modified T₀ plants are transferred to soil, and grow in a growth chamber or greenhouse under the suitable growth conditions. T₀ plants are phenotypically analyzed and grown for T₁ seed production by self-cross or back-cross to the maize WT.
g) Molecular analysis for genetic modification in the T1 progeny

Genome modification, for example, target genome editing in the genome of the bombarded immature embryo cells is likely inheritable. The inheritability of a genome editing event is determined by the segregation analysis of the modification in T₁ progeny. 10-20 days after pollination the immature T₁ embryos from the growing ear in a T₀ plant are isolated by direct pulling the embryos out of the kernels in the ear (starts from the tip of the ear) in planta (Note: DO NOT harvest the ears at this point and leave the ear with remaining kernels in T₀ plants for T₁ seed production). The DNA from each of the T₁ immature embryos are isolated individually, and the modification in T₁ progeny are analyzed by TaqMan qPCR, marker capillary electrophoresis analysis, and Digital Droplet PCR. Site-specific modification is further conformed by Sanger sequencing. The segregation analysis of the modification in T₁ progeny can also be performed using the mature T1 embryos, or the leaf tissues from the T1 seedlings.

The workflow was also demonstrated in FIG. 19. Using regeneration boosters as a “dual selection” a rapid plant regeneration and genome editing system was established. For maize it takes about 4-6 weeks from immature embryo to T0 plants, or 4-5 months from immature embryos to T1 seeds.

### Media

### Shooting Medium:

1×MS salt, 1×LS vitamins, 1×FeEDTA, 2.5 mg/L CuSO4.5H2O, 100 mg/L Myo Inosit, 5 mg/L Zeatin, 0.5 g/L of MES, 20 g/L of sucrose, 3 g/L Gelzan, pH: 5.8

### Rooting Medium:

1×MS salts, LS vitamins, 1×FeEDTA, 0.5 mg/L MES, 0.5 mg/L IBA, 1.25 mg/L of CuSO4, 20 g/L sucrose, 3 g/L Gelzan

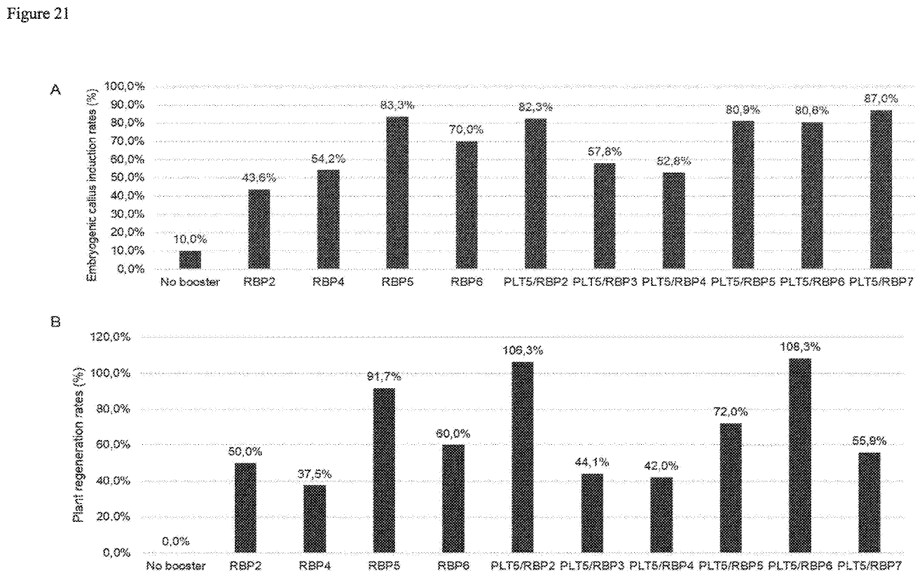

### Example 7: A Toolbox of Different Boosters Promote Embryogenic Callus Induction and Regeneration in Recalcitrant Elite 4V-40171 (5F1690, FIGS. 20 and 21)

For immature embryo isolation, osmotic treatment, bombardment, and embryogenic callus induction, please see the description in Example 1.

Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of maize elite 4V-40171 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

Embryogenic callus induction was observed under a Zeiss stereo microscope 4 days and 7 days after bombardment. The representative results were showed in FIG. 20.

Maize elite 4V-40171 is a recalcitrant genotype. Without a regeneration booster the immature embryos from this elite did not produce any regenerating structures when examined in both of 4 days and 7 days after bombardment (FIGS. 20A and H). Compared to the control without a booster, multiple embryogenic cell structures were induced from the embryos co-bombarded with different regeneration boosters (compare the results in FIG. 20 A to B-G and FIG. 20 H to K) in both of 4 days and 7 days after bombardment.

Callus induction rates were recorded 12 days after bombardment, which is defined as the numbers of embryos with at least one embryogenic callus from 100 embryos initially used. The callus induction rates were present in FIG. 21A, and plant regeneration rates (the numbers of regenerated T0 events from 100 embryos initially used) were presented in FIG. 21B. Without a booster this elite is generally a recalcitrant genotype, it became regenerative when a booster was applied (FIG. 21B) in our regeneration system. These results demonstrate that the boosters significantly promote cell proliferation and plant regeneration in maize elites.

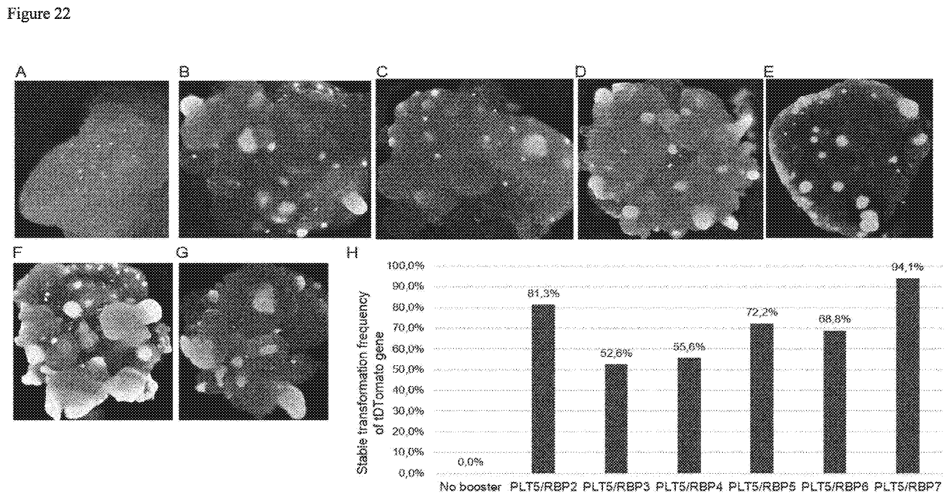

### Example 8: a Toolbox of Different Boosters Promote Rapid Transformation in Maize Recalcitrant Elite 4V-40171 (FIG. 22)

For immature embryo isolation, osmotic treatment, bombardment, and embryogenic callus induction, please see the description in Example 1.

Specifically, genome editing constructs pGEP359 (SEQ ID NO: 54, FIG. 13) and pGEP842 (SEQ ID NO: 53, FIG. 2) were co-bombarded with different boosters as indicated into the immature embryos of maize elite 4V-40171 by particle bombardment. For each bombardment (shot), 100 ng of plasmid pGEP359 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

tDTomato gene expression in the scutellum surface cells of the bombarded immature embryos was examined using a stereo fluorescence microscope at excitation 554 nm and emission 581 nm after bombardment and cultured in embryogenic callus induction for 8 days. The representative results are present in FIG. 22.

Without a booster the immature embryos from maize recalcitrant elite 4V-40171 did not produce a red fluorescent cell cluster (FIG. 22A). Contrast to the control without a booster, multiple embryogenic structures with strong and uniformed fluorescence signals appeared from the embryos co-bombarded with different regeneration boosters after bombardment and cultured for 8 days (FIG. 22B to G). Strong and uniformed expression of tDTomato in regenerated tissues, e.g. embryogenic callus, indicates the integration and stable transformation of this foreign gene. The stable transformation frequencies of tDTomato report gene in the co-bombarded immature embryos (defined as the numbers of embryos with at least one tDTomato-positive structure from 100 embryos initially used) were recorded 8 days after bombardment, and showed in FIG. 22H. As a recalcitrant genotype, the immature embryos from maize elite 4V-40171 is generally not responsible to transformation, but they became rapidly responsible and highly transformable when a booster was applied. The results demonstrate that boosters remarkably promote rapid stable transformation in maize elites and offer a positive selection for the transformed cells in cell proliferation and regeneration.

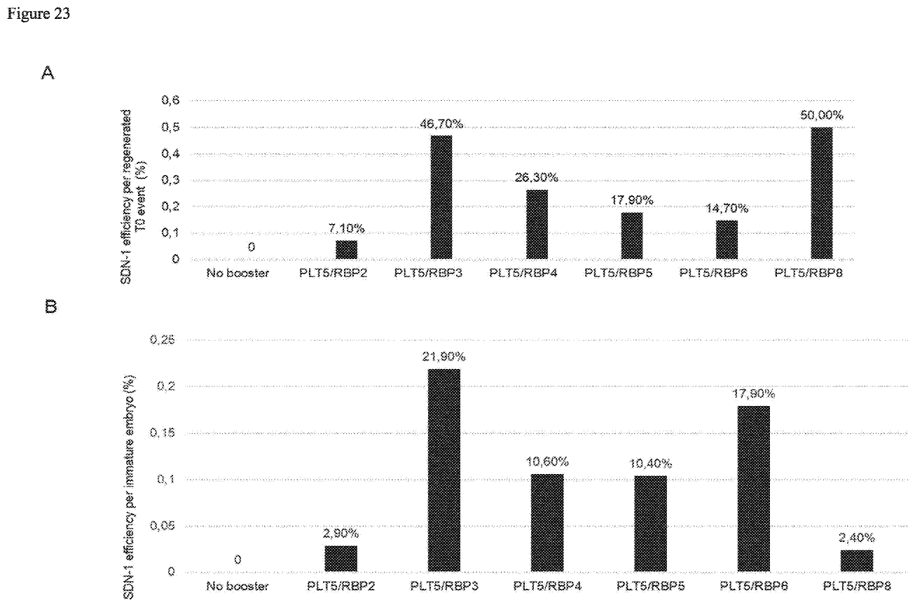

### Example 9: A Toolbox of Different Boosters Promote Genome Editing in T₀ Plants of Maize Recalcitrant Elite 4V-40171 (Table 3; FIG. 23).

The workflow was as described in Example 6. For immature embryo isolation, bombardment, and embryogenic callus induction post bombardment, please see the description in Example 1.

Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of maize elite 4V-40171 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

For embryogenic callus induction and plant regeneration and genome editing SDN-1 screening and confirmation, please see Example 5.

The immature embryos from maize recalcitrant elite 4V-40171 is generally not responsible to regeneration, but they became regenerative when a booster was co-delivered. An average ~20% of SDN-1 per regenerated T0 plant or 10% per immature embryo is achieved (Table 3; FIG. 23). The results demonstrate that boosters remarkably promote cell regeneration and genome editing in maize elites and offer a positive selection for the regeneration of transformed cells.

TABLE 3
Different boosters promote highly efficient genome
editing in recalcitrant maize elite 4V-40171

| | Total | Total | Bi- | Mon- | Total | % SDN-1/ | % SDN-1/ |
|---|---|---|---|---|---|---|---|
| Boosters | IE | Event | SDN-1 | SDN-1 | SDN-1 | Event | IE |
| No booster | 16 | 0 | 0 | 0 | 0 | 0 | 0 |
| PLT5/RBP2 | 34 | 14 | 1 | 0 | 1 | 7.1% | 2.9% |
| PLT5/RBP3 | 32 | 15 | 1 | 6 | 7 | 46.7% | 21.9% |
| PLT5/RBP4 | 47 | 19 | 3 | 2 | 5 | 26.3% | 10.6% |
| PLT5/RBP5 | 67 | 39 | 4 | 3 | 7 | 17.9% | 10.4% |
| PLT5/RBP6 | 56 | 68 | 4 | 6 | 10 | 14.7% | 17.9% |
| PLT5/RBP8 | 42 | 2 | 1 | 0 | 1 | 50.0% | 2.4% |
| Avg. with | 294 | 157 | 14 | 17 | 31 | 19.7% | 10.5% |
| boosters | | | | | | | |

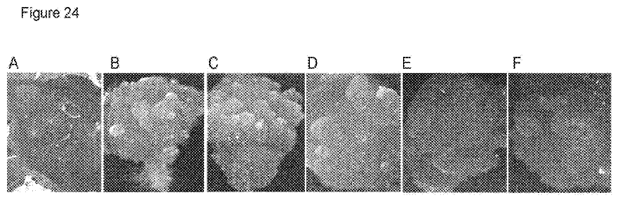

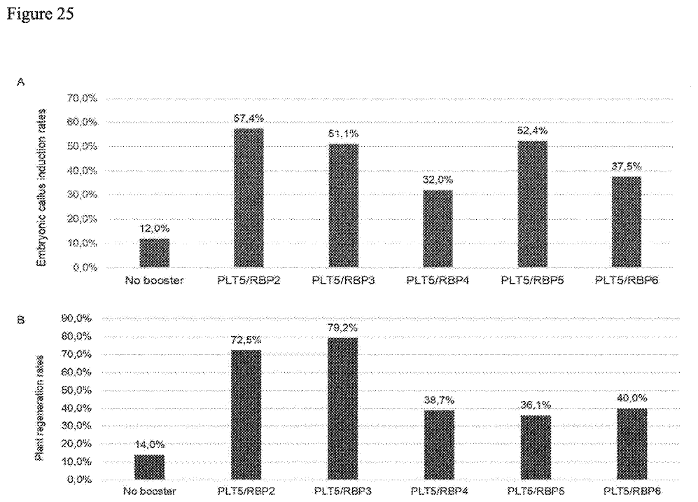

### Example 10: A Toolbox of Different Boosters Promote Embryogenic Callus Induction and Regeneration in Recalcitrant Elite 2V-20195 (FIGS. 24 and 25)

For immature embryo isolation, osmotic treatment, bombardment, and embryogenic callus induction, please see the description in Example 1 and Example 7.

Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of maize elite 2V-20195 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

Embryogenic callus induction was observed under a Zeiss stereo microscope 8 days after bombardment. The representative results were showed in FIG. 24. Without a regeneration booster majority of the immature embryos from the maize recalcitrant elite 2V-20195 did not produce regenerating structures in 8 days after bombardment (FIG. 24A). Compared to the control without a booster, multiple embryogenic cell structures were induced from the embryos co-bombarded with different regeneration boosters (compare the results in FIG. 24A to B-F) in both of 8 days after bombardment.

Callus induction rates were recorded 14 days after bombardment, which is defined as the numbers of embryos with at least one embryogenic callus from 100 embryos initially used. The callus induction rates were present in FIG. 25A, and plant regeneration rates (defined as the numbers of regenerated T0 events from 100 embryos initially used) were presented in FIG. 25B. Without a booster this elite had a low regeneration rate in both of callus induction and plant regeneration, and the both of regeneration rates increased significantly when a booster was co-delivered in our regeneration system (FIG. 25). These results demonstrate that the boosters significantly promote cell proliferation and plant regeneration in maize elites.

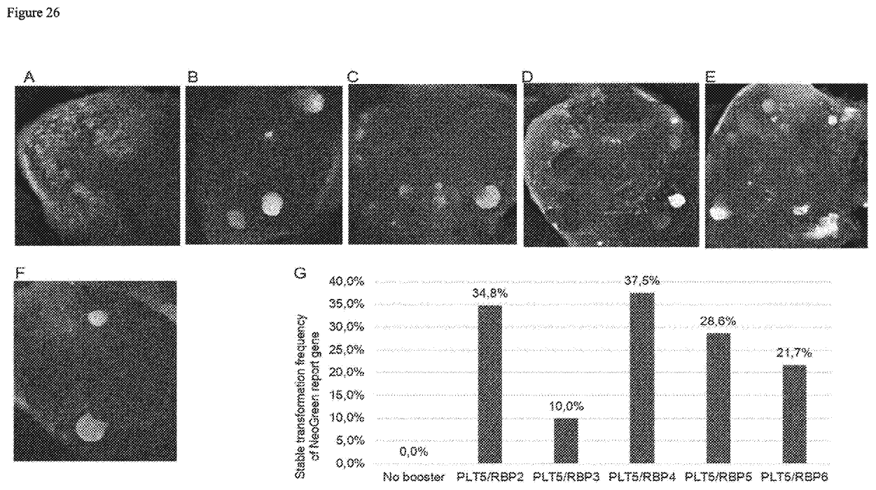

### Example 11: A Toolbox of Different Boosters Promote Rapid and Highly Efficient Genetic Transformation in Maize Recalcitrant Elite 2V-20195 (FIG. 26)

For immature embryo isolation, osmotic treatment, bombardment, and embryogenic callus induction, please see the description in Example 1, Example 7, and Example 10.

Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of maize elite 2V-20195 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

Green fluorescent report gene expression in the scutellum surface cells of the bombarded immature embryos was examined using a stereo fluorescence microscope at excitation maximum 506 nm and emission maximum 517 nm after bombardment and cultured in embryogenic callus induction for 4 days. The representative results are present in FIG. 26.

Without a booster the immature embryos from maize recalcitrant elite 2V-20195 did not produce a green fluorescent cell cluster (FIG. 26A). Contrast to the control without a booster, multiple embryogenic structures with strong and uniformed green fluorescence signals appeared from the embryos co-bombarded with different regeneration boosters after bombardment and cultured for 8 days (FIG. 26B to F). Strong and uniformed expression of green fluorescent report gene in regenerated tissues, e.g. embryogenic callus, indicates the integration and stable transformation of this foreign gene. The stable transformation frequencies of green fluorescent report gene in the co-bombarded immature embryos (defined as the numbers of embryos with at least one green fluorescent report gene-positive structure from 100 embryos initially used) were recorded 14 days after bombardment and showed in FIG. 26G. The immature embryos from maize elite 2V-20195 is generally not responsible to transformation, but they became rapidly responsible and highly transformable when a booster was applied. The results demonstrate that boosters remarkably promote rapid stable transformation in maize elites and offer a positive selection for the transformed cells in cell proliferation and regeneration.

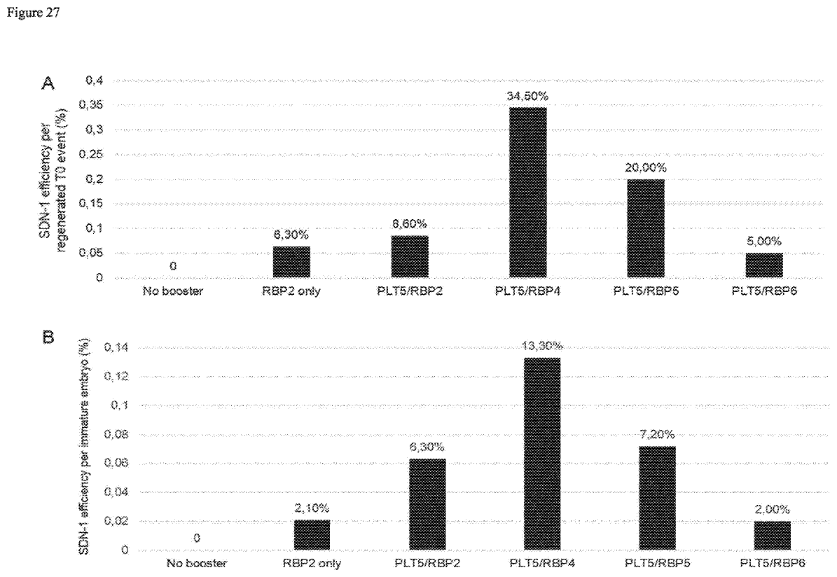

### Example 12: A Toolbox of Different Boosters Promote Rapid Genome Editing in T₀ Plants of Maize Recalcitrant Elite 2V-20195 (Table 4; FIG. 27)

The workflow was as described in Example 6. For immature embryo isolation, osmotic treatment, and bombardment, please see the description in Example 1. Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of maize elite 2V-20195 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

For plant regeneration, and genome editing SDN-1 screen and conformation in regenerated T0 plants please see Example 5 and Example 9.

The plant regeneration rate from elite 2V-20195 immature embryos is low in the control experiment without a booster. Only seven T0 plants were regenerated from 50 immature embryos initially bombarded. There was not any editing at the target site identified from the 7 regenerated plants (Table 4). The elite 2V-20195 immature embryos became more regenerative and more responsive to genome editing when a booster was co-delivered. An average 14.5% of SDN-1 per regenerated T0 plant or 6% of SDN-1 per immature embryo is achieved (Table 4; FIG. 27). The results reveal that the boosters promote cell regeneration and genome editing in maize elites and offer a positive selection for the regeneration of transformed cells.

TABLE 4
Different boosters promote genome editing in T0
plants of maize recalcitrant elite 2V-20195

| | Total | Total | Bi- | Mon- | Total | % SDN-1/ | % SDN-1/ |
|---|---|---|---|---|---|---|---|
| Boosters | IE | Event | SDN-1 | SDN-1 | SDN-1 | Event | IE |
| No booster | 50 | 7 | 0 | 0 | 0 | 0 | 0 |
| RBP2 only | 47 | 16 | 1 | 0 | 1 | 6.3% | 2.1% |
| PLT5/RBP2 | 80 | 58 | 2 | 3 | 5 | 8.6% | 6.3% |
| PLT5/RBP4 | 75 | 29 | 8 | 2 | 10 | 34.5% | 13.3% |
| PLT5/RBP5 | 97 | 35 | 6 | 1 | 7 | 20.0% | 7.2% |
| PLT5/RBP6 | 50 | 20 | 1 | 0 | 1 | 5.0% | 2.0% |
| Avg. with | 399 | 165 | 18 | 6 | 24 | 14.5% | 6.0% |
| boosters | | | | | | | |

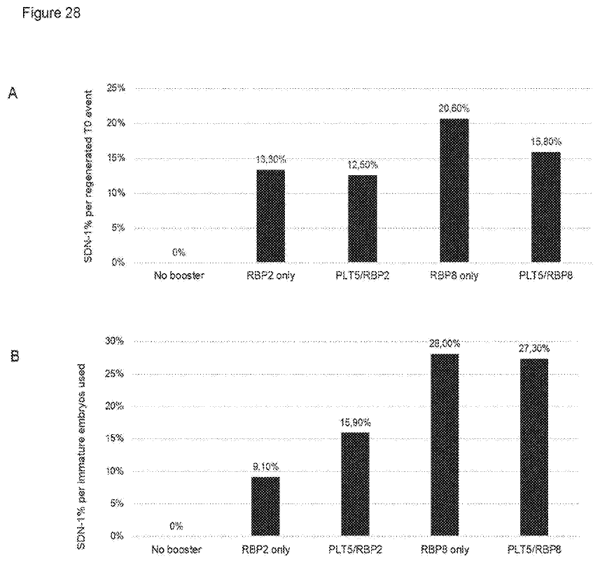

### Example 13: A Toolbox of Different Boosters Promote Rapid Genome Editing in T₀ Plants of Maize Recalcitrant Elite 4V-40290 (Table 5; FIG. 28)

The workflow was as described in Example 6. For immature embryo isolation, osmotic treatment, and bombardment, please see the description in Example 1. Specifically, genome editing constructs pGEP837 (SEQ ID NO: 52, FIG. 1) and pGEP842 (SEQ ID NO: 53, FIG. 2) were co-bombarded with the different boosters as indicated into the immature embryos of elite 4V-40290 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

For plant regeneration, and genome editing SDN-1 screen and conformation in regenerated T0 plants please see Example 5 and Example 9.

The plant regeneration rate from elite 4V-402 90immature embryos is low in the control experiment without a booster. Only five T0 plants were regenerated from 44 immature embryos initially bombarded. There was not any editing at the target site identified from the 5 regenerated plants (Table 5). The elite 4V-40290 immature embryos became more regenerative and more responsive to genome editing when a booster was co-delivered. An average 14% of SDN-1 per both of regenerated T0 plant and per immature embryo is achieved (Table 5; FIG. 28). The results reveal that the boosters promote cell regeneration and genome editing in maize elites and offer a positive selection for the regeneration of transformed cells.

TABLE 5
Different boosters promote rapid genome editing
in T₀ plants of maize recalcitrant elite 4V-40290

| | Total | Total | Bi- | Mon- | Total | % SDN-1/ | % SDN-1/ |
|---|---|---|---|---|---|---|---|
| Boosters | IE | Event | SDN-1 | SDN-1 | SDN-1 | Event | IE |
| No booster | 44 | 5 | 0 | 0 | 0 | 0% | 0% |
| RBP2 only | 22 | 15 | 2 | 0 | 2 | 13.3% | 9.1% |
| PLT5/RBP2 | 82 | 104 | 3 | 10 | 13 | 12.5% | 15.9% |
| RBP8 only | 25 | 34 | 1 | 6 | 7 | 20.6% | 28.0% |
| PLT5/RBP8 | 22 | 38 | 2 | 4 | 6 | 15.8% | 27.3% |
| Avg. with | 195 | 196 | 8 | 20 | 28 | 14.3% | 14.4% |
| boosters | | | | | | | |

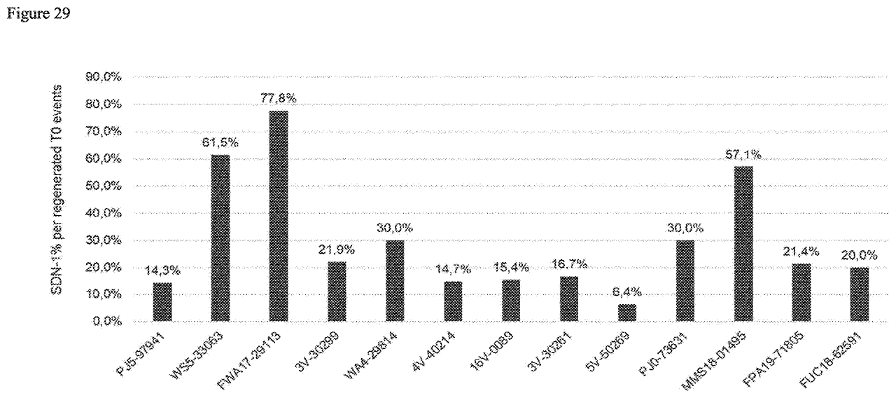

### Example 14: A Toolbox of Different KWS RBPs Enable Highly Efficient Genome Editing Directly in Maize Recalcitrant Elites (Table 6; FIG. 29)

To examine robustness and genotype-independence of using regeneration boosters as a “dual selection” for rapid regeneration and genome editing in maize, 13 additional maize recalcitrant elites were tested.

The workflow was as described in Example 6. For immature embryo isolation, osmotic treatment, and bombardment, please see the description in Example 1. Specifically, genome editing constructs pGEP837 (SEQ ID NO: 52, FIG. 1) and pGEP842 (SEQ ID NO_53, FIG. 2) were co-bombarded with the different boosters as indicated into the immature embryos of elite 4V-40290 by particle bombardment. For each bombardment (shot), 100 ng of plasmid pGEP837 and 150 ng of plasmid pGEP842 were co-coated with 100 ng each of different boost constructs onto 100 μg of 0.4 μm gold particles using calcium-spermidine method. Three bombardments (shots) per sample plate were performed. The bombarded embryos were transformed onto the embryogenic callus induction medium 16-20 hours after bombardment.

For plant regeneration, and genome editing SDN-1 screen and conformation in regenerated T0 plants please see Example 5 and Example 9.

An average genome editing SDN-1 efficiency per regenerated plants with a toolbox of different boosters for the 13 maize recalcitrant elites is list in Table 6, and also demonstrated in FIG. 29. The results further exhibit that the boosters promote cell regeneration and genome editing in maize elites and offer a positive selection for the regeneration of transformed cells. A toolbox of different boosters for highly efficient genome editing in maize recalcitrant elites was developed.

TABLE 6
Different boosters enable highly efficient
genome editing in directly in maize recalcitrant elites

| | Regen | Bi- | Mono- | Total | % |
|---|---|---|---|---|---|
| Elite ID | plant | SDN-1 | SDN-1 | SDN-1 | SDN-1 |
| PJ5-97941 | 56 | 7 | 1 | 8 | 14.3% |
| WS5-33063 | 13 | 6 | 2 | 8 | 61.5% |
| FWA17-29113 | 9 | 6 | 1 | 7 | 77.8% |
| 3V-30299 | 32 | 5 | 2 | 7 | 21.9% |
| WA4-29814 | 10 | 3 | 0 | 3 | 30.0% |
| 4V-40214 | 34 | 3 | 2 | 5 | 14.7% |
| 16V-0089 | 13 | 0 | 2 | 2 | 15.4% |
| 3V-30261 | 36 | 4 | 2 | 6 | 16.7% |
| 5V-50269 | 78 | 3 | 2 | 5 | 6.4% |
| PJ0-73631 | 63 | 13 | 6 | 19 | 30.0% |
| MMS18-01495 | 7 | 4 | 0 | 4 | 57.1% |
| FPA19-71805 | 14 | 0 | 3 | 3 | 21.4% |
| FUC18-62591 | 25 | 3 | 2 | 5 | 20.0% |

Example 15: A Toolbox of Different KWS RBPs Enable Highly Efficient Genome Editing with Single-Cell Origin Directly in Maize Recalcitrant Elites (Table 7).

Without a conventional selection for isolating and purifying the genetic modified cells during the regeneration chimeras may be an issue in the regenerated T0 plants. To address this matter the edited T0 events were analyzed by Sanger sequencing trace decomposition analysis of genome editing. The edited T0 events were generated from the co-bombardment of genome editing constructs of pGEP837 (SEQ ID NO: 52) and pGEP842 (SEQ ID NO: 53) with different boosters. For the workflow and methods to generate the T0 edited plants please see the descriptions in Examples above. The results revealed the majority of the edited T₀ plants containing one or two edits, suggesting a mono-allelic or bi-allelic SDN-1 event in the T₀ plants and the T₀ plants are likely regenerated from a single edited cell (the representative results were showed in Table 7).

TABLE 7
The representative results from sanger sequencing trace decomposition
analysis of genome editing SDN-1 T0 events in maize

| | | | event 1 | | | | | |
|---|---|---|---|---|---|---|---|---|
| SDN-1 ID | Genotype | % event 1 | detail | % event 2 | Event 2 | % event 3 | Event 3 | R square |
| xxxx008-T479 | A188 | 46.362 | 2 bp | 44.435 | wild | 1.579 | 1 bp | 0.960619 |
| | | | deletion | | type | | deletion | |
| xxxx008-T827 | A188 | 45.57 | wild | 43.69 | 5 bp | 2.248 | 5 bp | 0.960294 |
| | | | type | | deletion | | deletion | |
| xxxx011-T052 | A188 | 42.967 | wild | 41.21 | 20 bp | 6.149 | 20 bp | 0.955375 |
| | | | type | | deletion | | deletion | |
| xxxx011-T1163 | A188 | 44.017 | wild | 43.621 | 3 bp | 4.323 | 3 bp | 0.964498 |
| | | | type | | deletion | | deletion | |
| xxxx011-T1567 | A188 | 42.834 | wild | 42.457 | 2 bp | 4.133 | 3 bp | 0.964653 |
| | | | type | | deletion | | deletion | |
| xxxx011-T695 | A188 | 45.508 | 5 bp | 44.05 | 14 bp | 2.831 | 14 bp | 0.954624 |
| | | | deletion | | deletion | | deletion | |
| xxxx011-T731 | A188 | 43.83 | 4 bp | 42.935 | 6 bp | 3.247 | 6 bp | 0.966725 |
| | | | deletion | | deletion | | deletion | |
| xxxx011-T757 | A188 | 95.243 | 10 bp | 1.25 | 10 bp | 10.687 | 10 bp | 0.996223 |
| | | | deletion | | deletion | | deletion | |
| xxxx011-T850 | A188 | 44.982 | 9 bp | 43.588 | 23 bp | 1.68 | 9 bp | 0.951346 |
| | | | deletion | | deletion | | deletion | |
| xxxx021-T363 | A188 | 44.102 | 5 bp | 42.323 | 7 bp | 7.132 | 7 bp | 0.962038 |
| | | | deletion | | deletion | | deletion | |
| xx0245-T-358 | A188 | 98.595 | 5 bp | 0.784 | 5 bp | 0.113 | 7 bp | 0.998456 |
| | | | deletion | | deletion | | deletion | |
| xx0245-T-379 | A188 | 44.611 | 5 bp | 33.949 | 34 bp | 8.401 | 34 bp | 0.940991 |
| | | | deletion | | deletion | | deletion | |
| xxxx016-T018 | 4V-40290 | 62.07 | 8 bp | 32.566 | 8 bp | 1.342 | 28 bp | 0.958118 |
| | | | deletion | | deletion | | deletion | |
| xxxx016-T028 | 4V-40290 | 94.437 | 5 bp | 0.799 | 25 bp | 0.72 | 69 bp | 0.956733 |
| | | | deletion | | deletion | | deletion | |
| xxxx016-T106 | 4V-40290 | 49.409 | 3 bp | 41.984 | 2 bp | 1.124 | 2 bp | 0.955874 |
| | | | deletion | | deletion | | deletion | |
| xxxxx0254-T009 | PJ5-97941 | 39.42 | 29 bp | 36.296 | 11 bp | 6.505 | 11 bp | 0.949311 |
| | | | deletion | | deletion | | deletion | |
| xxxxx0254-T011 | PJ5-97941 | 39.327 | 12 bp | 35.518 | 8 bp | 5.555 | 8 bp | 0.958839 |
| | | | deletion | | deletion | | deletion | |
| xxxxx0254-T016 | PJ5-97941 | 43.116 | 8 bp | 39.967 | 6 bp | 4.636 | 8 bp | 0.958694 |
| | | | deletion | | deletion | | deletion | |
| xxxxx0254-T017 | PJ5-97941 | 44.7 | 5 bp | 44.557 | wild | 2.476 | 5 bp | 0.956408 |
| | | | deletion | | type | | deletion | |
| xxxxx0254-T032 | PJ5-97941 | 40.917 | 2 bp | 19.311 | 9 bp | 12.967 | 9 bp | 0.928388 |
| | | | deletion | | deletion | | deletion | |
| xxxxx0254-T037 | PJ5-97941 | 64.819 | 34 bp | 8.279 | 34 bp | 8.223 | 34 bp | 0.949046 |
| | | | deletion | | deletion | | deletion | |
| xx0255-T005 | WS5-33063 | 46.88 | wild | 36.695 | 7 bp | 7.569 | 7 bp | 0.954609 |
| | | | type | | deletion | | deletion | |
| xx0255-T006 | WS5-33063 | 44.519 | 7 bp | 18.525 | 12 bp | 18.215 | 12 bp | 0.964447 |
| | | | deletion | | deletion | | deletion | |

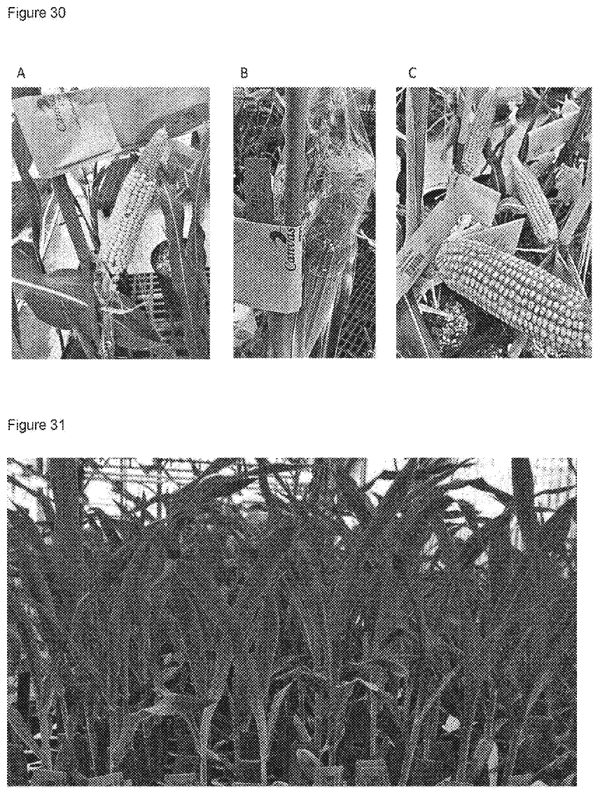

### Example 16: The Edited T₀ Plants are Normal, Fertile, and Transgene-Free (FIG. 30)

The edited T₀ plants were transferred into soil and growing in greenhouse for genotyping, phenotyping, and T1 seed production. The foreign DNA integration were analyzed by qPCR and WGS. T₀ plants are general normal and fertile (FIG. 30). There were only six T₀ plants showing foreign DNA integration from an analysis of total 642 regenerated T₀ plants, while no foreign DNA integration was detected from an analysis of 1471 T₁ progeny derived from 35 independent edited lines, and up to 99% of the edited T0 plants are transgene-free.

Example 17: The Edits are Full Inheritable from T0 to T1 Progeny (FIG. 31; Table 8)

T1 seeds are produced by selfing, which requires viable male and female gametes from T0 plants. T1 seed germination and growth in green house were normal (FIG. 31). Editing segregation in T1 progeny was analyzed by Taqman qPCR. A full match of the actual segregation of an editing event in the T1 progeny to the expected Mendel's Law of Segregation for a mono- or bi-allelic event was observed (Table 8). These results suggest that the edits are full transmittable/inheritable from T0 to T1 progeny, and also further support the idea of that the T0 plants are derived from single edited cells.

TABLE 8
The representative results from genome editing
SDN-1 segregation analysis in T1 progeny in the maize elites

| | | | SDN-1 in T1 | |
|---|---|---|---|---|
| Genotype | Event | SDN-1 in T0 | Total (WT:Mono-:Bi-) | |
| 4V-40171 | xxx223-T-050 | Bi-allelic | 20 | (0:0:20) |
| 4V-40171 | xxx230-T-088 | Bi-allelic | 20 | (0:0:20) |
| 4V-40171 | xxx232-T-077 | Mono-alleic | 20 | (5:8:7) |
| 4V-40171 | xxx232-T-103 | Mono-alleic | 19 | (1:13:5) |
| 2V-20195 | xxx231-T-006 | Mono-alleic | 16 | (2:10:4) |
| 2V-20195 | xxx231-T-071 | Mono-alleic | 15 | (4:7:4) |
| 2V-20195 | xxx231-T-082 | Bi-allelic | 18 | (0:0:18) |
| 2V-20195 | xxx231-T-113 | Bi-allelic | 17 | (0:0:17) |
| 4V-40290 | xxxD004-T016 | Bi-allelic | 20 | (0:0:20) |
| 4V-40290 | xxxD004-T031 | Bi-allelic | 20 | (0:0:20) |
| 4V-40290 | xxxD004-T086 | Mono-llelic | 19 | (3:10:6) |
| 4V-40290 | xxxD006-T085 | Mono-allelic | 19 | (7:8:4) |
| PJ5-97941 | xxx254-T-009 | Bi-allelic | 8 | (0:0:8) |
| PJ5-97941 | xxx254-T-016 | Bi-allelic | 19 | (0:0:19) |
| PJ5-97941 | xxx254-T-017 | monoallelic | 9 | (2:3:3) |
| PJ5-97941 | xxx254-T-041 | Biallelic (back- | 7 | (3:4:0) |

cross to WT)

Example 18: Co-Delivery of Trichostatin A (TSA) with Genome Editing Constructs

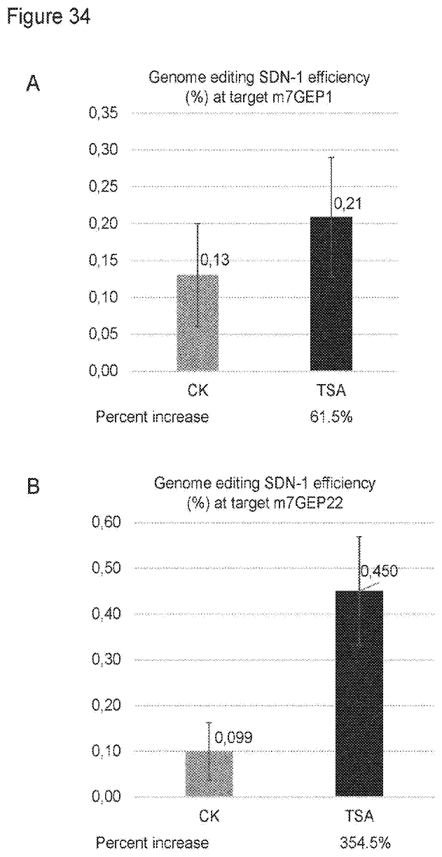

The co-delivery by microprojectile bombardment increased genome editing SDN-1 efficiency in corn A188 immature embryos (FIG. 34).

### Procedure:

For immature embryo isolation, bombardment, and embryogenic callus induction post bombardment, please see the description in Example 1.

The amounts of TSA used for a bombardment with 100 μg of gold particles (approximately, 4.0-5.0×10⁷ gold particles size of 0.6 micron) is 15 ng. Specifically, the plasmid DNA and TSA were co-coated onto gold particles for bombardment as follows. For 10 shots, 100 microlite (μl) of 10 mg/ml 0.6 μm gold particle in 50% (v/v) glycerol particle stock solution (1.0 mg of the gold particles in total for 10 shots, and 100 μg per shot) was pipetted into a clear low-retention microcentrifuge tube. Sonicate for 15 seconds to suspend the gold particles. While vortex at a low speed, add the following in order:
1.0 μg of pGEP1054 (FIG. 15), 1.5 μg of pGEP842 (FIG. 2) or pGEP1067 (FIG. 32)
100 μl of 2.5 M CaCl₂ (pre-cold on ice)
40 μl of 0.1 M cold spermidine

Close the lid and vortex the tube for 5 minutes at room temperature and spin down the DNA-coated gold particles. After washing in 1000 μl of 100% ethanol (pre-cold at −20° C.) for two times, the pellet was resuspended in 120 μl of 100% ethanol. Finally, 150 ng of TSA was added into the re-suspended gold particle solution carefully. While vertexing at a low speed, pipet 10 μl of TSA co-coated gold particles with a wide open 20 μl tip from the tube onto the center of the macrocarrier evenly. Since the particles tend to form clumps at this point, get the gold particles onto the macrocarriers as soon as possible. Brief air dry in a clear laminar hood.

Bombardment was conducted using a Bio-Rad PDS-1000/He particle gun. The bombardment conditions are: 28-30 mm/Hg vacuum, 450 psi rupture disc, 6 mm gap distance, the specimen platform is in the second position from the bottom in the chamber at a distance of 60 mm, and three shots per sample plate. After bombardment the embryos were remained on the osmotic plate for another 16 hours, and then removed onto an embryogenic callus induction medium plate (N6_5Ag). 48 hours after bombardment, the bombarded embryos were sampled for DNA extraction and genome editing SDN-1 analysis by Taqman ddPCR.

Co-delivery of 15 ng TSA and genome editing components in corn immature embryos by microprojectile bombardment significantly improves the genome editing SDN-1 efficiency at two CRISPR target sites from 6 repeats. Compared to the control without TSA (CK), a 61.5% (FIG. 34A) and 354.5% (FIG. 34 B) increase in the SDN-1 efficiency was detected at the target m7GEP1 and m7GEP22, respectively. These results clearly demonstrate the epigenetically regulating chemical, specifically the histone deacetylase inhibitor (HDACI) trichostatin A (TSA) enhances genome editing SDN-1 efficiency, which might be achieved through its roles in relaxing chromatin and increasing the target accessibility.

1-26. (canceled)

27. A method for plant genome modification of at least one genomic target sequence, for obtaining at least one modified cell wherein the method comprises the following steps:
(a) providing at least one plant cell or a single plant cell;
(b) introducing into the at least one plant cell or the single plant cell:
(i) at least one genome modification system comprising at least one nucleic acid guided nuclease, nickase or an inactivated nuclease, or a sequence encoding the same, and optionally at least one guide molecule, or a sequence encoding the same;
(ii) at least one regeneration booster, or a sequence encoding the same and/or at least one epigenetically regulating chemical, wherein the at least one regeneration booster is transiently present, transiently active or transiently expressed in the plant cell;
(iii) and, optionally at least one repair template, or a sequence encoding the same; and
(c) cultivating the at least one plant cell or the single plant cell under conditions allowing the expression and/or assembly and/or activation of the at least one genome modification system, and, optionally of the at least one regeneration booster, and optionally of the at least one guide molecule and/or optionally of the at least one repair template; and
(d) obtaining at least one modified plant cell; and/or
(e) obtaining at least one plant tissue, organ, plant or seed regenerated from the at least one modified cell; and
(f) optionally: screening for at least one plant tissue, organ, plant or seed regenerated from the at least one modified cell in the T0 and/or T1 generation carrying a desired targeted modification,
wherein the at least one regeneration booster comprises at least one regeneration booster protein (RBP), wherein the at least one RBP comprises the amino acid sequence of SEQ ID NO: 39, or wherein the at least one RBP is encoded by the nucleic acid sequence of SEQ ID NO: 27, or a cognate codon-optimized sequence.

28. The method of claim 27, wherein steps (i) and (ii) take place simultaneously or subsequently, for promoting plant cell proliferation and/or to assist in a targeted modification of at least one genomic target sequence.

29. The method of claim 27, wherein at least one regeneration booster is introduced in step (ii) and
(a) the regeneration booster(s) promote plant cell proliferation and/or assist in the targeted modification and/or provide a positive selection for at least one transiently transformed cell for genome modification and/or for regeneration into at least one modified plant, and/or
(b) the regeneration booster(s) suppress plant cell differentiation if the cell is stably transformed and provide a negative selection for at least one stably transformed cell for regeneration into one transgenic plant, and/or
(c) the regeneration booster(s) provide a dual selection according to (a) and (b) for at least one transformed cell.

30. The method of claim 27, wherein the at least one plant cell is a cell of a zygotic or somatic embryo or meristem or wherein the single plant cell is a diploid cell or a haploid cell.

31. The method of claim 27, wherein the at least one regeneration booster further comprises at least one PLETHORA (PLT), wherein the at least one PLT comprises the amino acid sequence of SEQ ID NO: 30 or 31, or wherein the at least one PLT is encoded by the nucleic acid sequence of SEQ ID NO: 18 or 19, or a cognate codon-optimized sequence.

32. The method of claim 27, wherein at least one further regeneration booster is introduced, wherein the further regeneration booster, or the sequence encoding the same comprises babyboom (BBM), WUSCHEL (WUS), WUSCHEL-related homeobox (WOX), growth-regulating factor (GRF), and leafy cotyledon (LEC), or a variant thereof or wherein the further regeneration booster is selected from RBP1 to RBP8, and wherein the at least one further regeneration booster is different from the at least one regeneration booster.

33. The method of claim 27, wherein at least one epigenetically regulating chemical is introduced in step ii) and wherein the at least one epigenetically regulating chemical is a histone deacetylase inhibitor (HDACI), trichostatin A (TSA) or a TSA like chemical.

34. The method of claim 27, wherein the at least one genome modification system, and the at least one regeneration booster, or the sequences encoding the same and/or the at least one epigenetically regulating chemical, are introduced into the cell by transformation or transfection mediated by biolistic bombardment, Agrobacterium-mediated transformation, micro- or nanoparticle delivery, or by chemical transfection, or a combination thereof, wherein when the at least one genome modification system, and the at least one regeneration booster and/or the at least one epigenetically regulating chemical are introduced by biolistic bombardment, the biolistic bombardment comprises a step of osmotic treatment before and/or after bombardment.

35. The method of claim 27, wherein the at least nucleic acid guided nuclease, nickase or an inactivated nuclease, or a sequence encoding the same, is selected from the group consisting of a CRISPR/MAD7 system, a CRISPR/Cfp1 system, a CRISPR/MAD2 system, a CRISPR/Cas9 system, a CRISPR/CasX system, a CRISPR/CasY system, a CRISPR/Cas13 system, or a CRISPR/Csm system, a zinc finger nuclease system, a transcription activator-like nuclease system, and a meganuclease system, or any combination or variant thereof.

36. The method of claim 27, wherein the at least one genome modification system further comprises at least one reverse transcriptase and/or at least one cytidine or adenine deaminase, wherein the at least one cytidine or adenine deaminase is independently selected from an apolipoprotein B mRNA-editing complex (APOBEC) family deaminase, an activation-induced cytidine deaminase (AID), an ACF1/ASE deaminase, an ADAT family deaminase, an ADAR2 deaminase, or a PmCDA1 deaminase, a TadA derived deaminase, and/or a transposon, or a sequence encoding the aforementioned at least one enzyme, or any combination or variant thereof.

37. The method of claim 27, wherein the at least one genome modification system comprises at least one repair template, and wherein the at least one repair template comprises or encodes a double- and/or single-stranded nucleic acid sequence.

38. The method of claim 37, wherein the at least one repair template comprises symmetric or asymmetric homology arms and/or wherein the at least one repair template comprises at least one chemically modified base and/or backbone.

39. The method of claim 27, wherein the at least one genome modification system, and optionally the at least one repair template, or the respective sequences encoding the same, are introduced transiently or stably, or as a combination thereof.

40. A method of using a first regeneration booster or a combination of regeneration boosters, in a method for targeted plant genome modification according to claim 27, to
(a) promote plant cell proliferation and/or assist in the targeted modification and/or provide a positive selection for at least one transiently transformed cell for genome modification and/or for regeneration into at least one modified plant, and/or
(b) suppress plant cell differentiation if the cell is stably transformed and provide a negative selection for at least one stably transformed cell for regeneration into one transgenic plant, and/or
(c) provide a dual selection according to (a) and (b) for at least one transformed cell, wherein the first regeneration booster or combination of regeneration boosters comprise:
(i) at least one RBP, wherein the at least one RBP comprises the amino acid sequence of SEQ ID NO: 39, or wherein the at least one RBP is encoded by the nucleic acid sequence SEQ ID NO: 27, or a cognate codon-optimized sequence, and/or
(ii) at least one PLT, wherein the at least one PLT comprises the amino acid sequence of SEQ ID NO: 30 or 31, or wherein the at least one PLT is encoded by the nucleic acid sequence of SEQ ID NO: 18 or 19, or a cognate codon-optimized sequence, and/or
(iii) at least one further regeneration booster, wherein the further regeneration booster, or the sequence encoding the same comprises BBM, WUS, WOX, GRF, LEC, or a variant thereof or wherein the further regeneration booster selected from RBP1 to RBP8, wherein the further regeneration booster is different from the first regeneration booster.

41. A method for selecting a modified plant cell, plant tissue, organ, plant or seed, wherein the method comprises the steps (a) to (c) as defined in claim 27 and further comprises the step:
(d1) screening for at least one plant cell carrying a targeted modification, or
(d2) screening for at least one plant tissue, organ, plant or seed regenerated from at least one modified plant cell in the T0 or T1 generation carrying a desired targeted modification.

42. A method for regenerating a plant tissue, organ or plant from at least one plant cell or a single plant cell, wherein the method comprises:
(i) introducing into the at least one plant cell or the single plant cell at least one regeneration booster, or a sequence encoding the same, wherein the at least one regeneration booster is transiently present, transiently active or transiently expressed in the plant cell, and
(ii) regenerating a plant tissue, organ or plant from at least one plant cell or the single plant cell, wherein the at least one regeneration booster comprises:
(a) at least one RBP, wherein the at least one RBP comprises the amino acid sequence of SEQ ID NO: 39, or wherein the at least one RBP is encoded by the sequence of SEQ ID NO: 27, or a cognate codon-optimized sequence, and/or
(b) at least one PLT, wherein the at least one PLT comprises the amino acid sequence of SEQ ID NO: 30 or 31, or wherein the at least one PLT is encoded by the sequence of SEQ ID NO: 18 or 19, or a cognate codon-optimized sequence, and/or
(c) at least one further regeneration booster, wherein the further regeneration booster, or the sequence encoding the same is selected from BBM, WUS, WOX, GRF, LEC, or a variant thereof or wherein the further regeneration booster selected from RBP1 to RBP8, wherein the further regeneration booster is different from the first regeneration booster.

43. The method of claim 42, wherein the at least one plant cell is an immature embryo cell or meristematic cell, in particular a cell of a zygotic or somatic embryo or meristem wherein the single plant cell is a diploid cell or a haploid cell.

44. The method of claim 43, wherein the single plant cell is a haploid microspore.

45. A plant cell, tissue, organ, plant or seed obtainable by or obtained by a method according to claim 27.

46. The plant cell, tissue, organ, plant or seed according to claim 45, wherein the plant cell, tissue, organ, plant or seed originates from a monocotyledonous or a dicotyledonous plant.

47. The plant cell, tissue, organ, plant or seed according to claim 46, wherein the plant cell, tissue, organ, plant or seed originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant cell, tissue, organ, plant or seed originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanfolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.
